(19) United States
(12) Patent Application Publication
(10) Pub. No.: US 2024/0148269 A1
(43) Pub. Date: May 9, 2024

(54) INTERROGATION AND DETECTION SYSTEMS FOR RADIO-FREQUENCY TAGS, AND METHODS

(71) Applicant: Covidien LP, Mansfield, MA (US)

(72) Inventors: Edward L. Brannan, Erie, CO (US); Aaron G. Mattmiller, Berthoud, CO (US)

(21) Appl. No.: 18/279,092

(22) PCT Filed: Jan. 26, 2022
(86) PCT No.: PCT/US2022/013842

§ 371 Date: Aug. 28, 2023

Related U.S. Application Data
(60) Provisional application No. 63/157,044, filed on Mar. 5, 2021.
(60) Provisional application No. 63/157,050, filed on Mar. 5, 2021.
(60) Provisional application No. 63/157,054, filed on Mar. 5, 2021.
(60) Provisional application No. 63/157,058, filed on Mar. 5, 2021.
(60) Provisional application No. 63/157,063, filed on Mar. 5, 2021.
(60) Provisional application No. 63/157,065, filed on Mar. 5, 2021.

(51) Int. Cl. A61B 5/06; A61B 90/98; G06K 7/10

(52) U.S. Cl. CPC A61B 5/062; A61B 90/98; G06K 7/10336

(57) ABSTRACT

An interrogation and detection system for detection of surgical implements within a patient's body is presented. The system includes an RFID tag configured to transmit a return signal when energized, a signal generator configured to generate an energizing signal for the RFID tag, and a coil antenna operably coupled to the signal generator, the antenna configured to receive a return signal transmitted by the RFID tag. The coil antenna includes a primary coil, a secondary coil, and a core configured to couple electromagnetic energy from the secondary coil to the primary coil. The core includes a non-magnetic insulating material. The secondary coil is configured to receive the return signal. The primary coil is configured to couple electromagnetic energy from the secondary coil. The RFID tag affixed to a surgical implement.

### CROSS-REFERENCE TO RELATED APPLICATIONS

The present application is a U.S. National Stage Application filed under 35 U.S.C. § 371(a) of International Patent Application No. PCT/US2022/013482, filed on Jan. 26, 2022, which claims benefit of and priority to each of U.S. Provisional Patent Application Nos. 63/157,044 filed on Mar. 5, 2021, 63/157,050 filed on Mar. 5, 2021, 63/157,054 filed on Mar. 5, 2021, 63/157,058 filed on Mar. 5, 2021, 63/157,063 filed on Mar. 5, 2021 and 63/157,065 filed on Mar. 5, 2021. The entire disclosures of the foregoing applications are incorporated by reference herein.

### FIELD

This disclosure relates generally to interrogation and detection systems for radio-frequency (RF) tags, and more particularly, interrogation, detection and inventory systems for radio-frequency (RF) tags for use within surgical sites.

### BACKGROUND

It is often useful to determine whether objects associated with a surgery are present in a patient's body before completion of the surgery. Such objects may take a variety of forms. For example, the objects may take the form of instruments, for instance, scalpels, scissors, forceps, hemostats, and/or clamps. Also, for example, the objects may take the form of related accessories and/or disposable objects, for instance, surgical sponges, gauzes, and/or pads. Failure to locate an object before closing the patient may require additional surgery, and in some instances, may have unintended medical consequences.

Accordingly, there is a need for a technology that is capable of providing both presence detection and tagged surgical item/implement identification functionality in the medical setting, as well as inventory controls of the tagged items/implements. Specifically, detecting the presence of, identifying, and maintaining inventory of tagged surgical items and materials that are used during the execution of a medical procedure. Technologies exist that enable these functions both individually as well as in conjunction with each other, but the methods and packaging of the discrete solutions used are not ideal for the application. More specifically, the components attached or affixed to the items being tracked are either too large physically and present nuisances or obstacles in the execution of the procedure, or the detection and identification performance of the solution may degrade rapidly in the presence of variable and uncontrolled dielectric or conductive materials.

Accordingly, there are needs for improvements in presence detection, tagged item identification, and inventory functionality in the medical setting.

### SUMMARY

This disclosure relates to systems for detection of surgical objects and devices used in body cavities during surgery, specifically antennae to be inserted directly into a surgical site.

In accordance with aspects of the disclosure, an interrogation and detection system for detection of surgical implements within a patient's body is presented. The system includes an RFID tag configured to transmit a return signal when energized, a signal generator configured to generate an energizing signal for the RFID tag, and a coil antenna operably coupled to the signal generator, the antenna configured to receive a return signal transmitted by the RFID tag. The coil antenna includes a primary coil, a secondary coil, and a core configured to couple electromagnetic energy from the secondary coil to the primary coil. The core includes a non-magnetic insulating material. The secondary coil is configured to receive the return signal. The primary coil is configured to couple electromagnetic energy from the secondary coil. The RFID tag affixed to a surgical implement.

In an aspect of the present disclosure, a “turns ratio” between the primary coil and the secondary coil may be greater than or equal to 1:1. The “turns ratio” is a ratio between a first quantity of turns of the primary coil and a second quantity of turns of the secondary coil.

In another aspect of the present disclosure, the secondary coil may include a resonant frequency within 10% of an operating frequency of the return signal.

In yet another aspect of the present disclosure, the secondary coil may include an inductance greater than or equal to 2.5 uH.

In a further aspect of the present disclosure, the coil antenna may further include a first matching network electrically connected to the primary coil. The first matching network is configured to match an input impedance of the primary coil to an output impedance of the generator.

In yet a further aspect of the present disclosure, the coil antenna may further include a second matching network electrically connected to the secondary coil. The second matching network is configured to match an input impedance of the secondary coil to an output impedance of the primary coil.

In an aspect of the present disclosure, the primary coil may be a first planar coil.

In another aspect of the present disclosure, the secondary coil may be a second planar coil.

In yet another aspect of the present disclosure, the primary coil may include a first turn and a second turn.

In a further aspect of the present disclosure, the first turn of the primary coil and the second turn of the primary coil may be arranged in an offset manner in a vertical and a horizontal orientation.

In yet a further aspect of the present disclosure, the secondary coil may include a first turn and a second turn.

In an aspect of the present disclosure, the first turn of the primary coil and the second turn of the primary coil may be arranged in an offset manner in a vertical and a horizontal orientation.

In accordance with aspects of the disclosure, a coil antenna configured to receive a return signal transmitted by the RFID tag is presented. The coil antenna includes a primary coil, a secondary coil, and a core configured to couple electromagnetic energy from the secondary coil to the primary coil. The core includes a non-magnetic insulating material. The secondary coil is configured to receive the return signal. The primary coil is configured to couple electromagnetic energy from the secondary coil.

In another aspect of the present disclosure, the primary coil and the secondary coil may each be planar coils.

In yet another aspect of the present disclosure, the primary coil may include a conductor.

In a further aspect of the present disclosure, the conductor of the primary coil includes a conductor that may have a configuration including coaxial, planar, a “C” shaped transverse cross-sectional shape, and/or tube-shaped transverse cross-sectional shape.

In yet a further aspect of the present disclosure, a “turns ratio” between the primary coil and the secondary coil may be greater than or equal to 1:1. The “turns ratio” is a ratio between a first quantity of turns of the primary coil and a second quantity of turns of the secondary coil.

In an aspect of the present disclosure, the secondary coil may include a resonant frequency within 10% of an operating frequency of the return signal.

In accordance with aspects of the disclosure, a method for inventory control of tagged items is presented. The method includes transmitting an energizing signal by a coil antenna operably coupled to a signal generator, the coil antenna including a primary coil, a secondary coil, and a core configured to magnetically couple the secondary coil to the primary coil, the energizing signal configured to energize an RFID tag affixed to an item and configured to transmit a return signal when energized and receiving a return signal, by a primary coil of the coil antenna.

In yet a further aspect of the present disclosure, the method further may include detecting and/or identifying the item based on the return signal. The item may include a surgical implement.

In accordance with aspects of the disclosure, a coil configured to receive a return signal transmitted by an RFID tag, includes a first turn conductor and a second turn conductor electrically connected to the first turn conductor.

In an aspect, the first turn conductor may be located in parallel relation and an offset manner to the second turn conductor.

In an aspect of the present disclosure, the first turn conductor may include a first inner edge and a first outer edge. The second turn conductor may include a second inner edge and a second outer edge.

In another aspect of the present disclosure, the coil may be arranged in a circular, square, rectangular, or oblong configuration.

In yet another aspect of the present disclosure, the first turn conductor may overlap the second turn conductor of the staggered coil, where a substantial portion of the second turn conductor is not overlapped by the first turn conductor.

In accordance with aspects of the disclosure, coil antenna configured to receive a return signal transmitted by an RFID tag, includes a primary coil and a secondary coil. The secondary coil includes a first turn conductor and a second turn conductor electrically connected to the first turn conductor. The secondary coil is configured to receive the return signal. The primary coil is configured to couple electromagnetic energy from the secondary coil.

In a further aspect of the present disclosure, the coil antenna may further include a core configured to couple electromagnetic energy to and from the secondary coil to the primary coil The core includes a non-magnetic insulating material.

In yet a further aspect of the present disclosure, the first turn conductor may be located in parallel relation and an offset manner to the second turn conductor.

In yet a further aspect of the present disclosure, a “turns ratio” between the primary coil and the secondary coil may be greater than or equal to 1:1. The “turns ratio” is a ratio between a first quantity of turns of the primary coil and a second quantity of turns of the secondary coil.

In an aspect of the present disclosure, the secondary coil may include a resonant frequency within 10% of an operating frequency of the return signal.

In another aspect of the present disclosure, the secondary coil may include an inductance greater than or equal to 2.5 uH.

In yet another aspect of the present disclosure, the coil antenna may further include a first matching network electrically connected to the primary coil. The first matching network may be configured to match an input impedance of the primary coil to an output impedance of the signal generator.

In accordance with aspects of the disclosure, an interrogation and detection system for the detection of surgical implements within a patient's body is presented. The interrogation and detection system includes an RFID tag configured to transmit a return signal when energized, a signal generator configured to generate an energizing signal for the RFID tag, and a coil antenna operably coupled to the signal generator, the antenna configured to receive a return signal transmitted by the RFID tag. The RFID tag is affixed to a surgical implement within the patient's body. The coil antenna includes a primary coil and a secondary coil. The secondary coil includes a first turn conductor and a second turn conductor electrically connected to the first turn conductor. The first turn conductor may be located in parallel relation and an offset manner to the second turn conductor. The secondary coil is configured to receive the return signal. The primary coil is configured to couple electromagnetic energy from the secondary coil.

In a further aspect of the present disclosure, the secondary coil may be air-core coupled to the primary coil.

In yet a further aspect of the present disclosure, a “turns ratio” between the primary coil to the secondary coil may be greater than or equal to 1:1. The “turns ratio” is a ratio between a first quantity of turns of the primary coil and a second quantity of turns of the secondary coil.

In accordance with aspects of the disclosure, the secondary coil may include an inductance greater than or equal to 2.5 uH.

In an aspect of the present disclosure, the coil antenna may further include a first matching network electrically connected to the primary coil.

In another aspect of the present disclosure, the coil antenna may further include a second matching network electrically connected to the secondary coil.

In yet another aspect of the present disclosure, the primary coil may be a first planar coil.

In a further aspect of the present disclosure, the secondary coil may be a second planar coil.

In yet a further aspect of the present disclosure, the primary coil may include two or more turns.

In an aspect of the present disclosure, the secondary coil may include two or more turns.

In accordance with aspects of the disclosure, an interrogation and detection system for detection of surgical implements within a patient's body, is presented. The interrogation and detection system includes an RFID tag configured to transmit a return signal when energized, a signal generator configured to generate an energizing signal for the RFID tag, and a coil antenna operably coupled to the signal generator, the antenna configured to receive a return signal transmitted by the RFID tag, the coil antenna including a coil array. The RFID tag affixed to a surgical implement within the patient's body.

In another aspect of the present disclosure, the coil array may be configured to generate a magnetic flux and steer a direction of the magnetic flux and/or a magnitude of the magnetic flux based on the energizing signal.

In yet another aspect of the present disclosure, the coil array includes a first coil and a second coil. The energizing signal may include a first current and a second current. The first coil and the second coil may be configured to independently be energized by the first current and the second current, respectively.

In a further aspect of the present disclosure, the second coil may be oriented 0, 90, 180, and/or 270 degrees relative to the first coil.

In yet a further aspect of the present disclosure, the first coil of the coil array may be energized with the first current in a clockwise direction and/or a counter-clockwise direction. The second coil of the coil array is energized with the second current in a clockwise direction and/or a counter-clockwise direction.

In accordance with aspects of the disclosure, each coil of the coil array may be arranged in a circular, square, rectangular, and/or oblong configuration.

In an aspect of the present disclosure, each of the first coil and the second coil may be planar coils.

In another aspect of the present disclosure, each of the first coil and the second coil may include one or more turns.

In yet another aspect of the present disclosure, each of the coils of the coil array may include a primary coil and a secondary coil. Each of the coils of the coil array may further include a “turns ratio” between the primary coil to the secondary coil greater than or equal to 1:1. The “turns ratio” is a ratio between a first quantity of turns of the primary coil and a second quantity of turns of the secondary coil.

In accordance with aspects of the disclosure, a coil array configured to receive a return signal transmitted by an RFID tag, includes a first coil configured to generate a first magnetic field and a second coil configured to generate a second magnetic field. The coil array is configured to generate a magnetic flux and steer a direction of the magnetic flux and/or a magnitude of the magnetic flux based on an energizing signal from a signal generator.

In a further aspect of the present disclosure, the energizing signal may include a first current and a second current. The first coil and the second coil may be configured to independently be energized by the first current and the second current, respectively.

In yet a further aspect of the present disclosure, the second coil may be oriented 0, 90, 180, and/or 270 degrees relative to the first coil.

In an aspect of the present disclosure, the first coil of the coil array may be energized with the first current in a clockwise direction and/or a counter-clockwise direction. The second coil of the coil array may be energized with the second current in a clockwise direction and/or a counter-clockwise direction.

In another aspect of the present disclosure, each coil of the coil array may be arranged in a circular, square, rectangular, and/or oblong configuration.

In yet another aspect of the present disclosure, each of the first coil and the second coil may be planar coils.

In a further aspect of the present disclosure, each of the first coil and the second coil may include one or more turns.

In yet a further aspect of the present disclosure, each of the coils of the coil array may include a primary coil and a secondary coil. Each of the coils of the coil array may further include a “turns ratio” between the primary coil to the secondary coil greater than or equal to 1:1. The “turns ratio” is a ratio between a first quantity of turns of the primary coil and a second quantity of turns of the secondary coil.

In accordance with aspects of the disclosure, each of the coils of the coil array may include an inductance greater than or equal to 2.5 uH.

In an aspect of the present disclosure, a method for interrogation and detection of surgical implements within a patient's body is presented. The method includes transmitting an energizing signal by a coil antenna operably coupled to a signal generator, the antenna configured to receive a return signal transmitted by an RFID tag, the coil antenna including a coil array, and receiving a return signal, by the coil array. The coil array is configured to generate a magnetic flux and steer a direction of the magnetic flux and/or a magnitude of the magnetic flux based on the energizing signal.

In another aspect of the present disclosure, the energizing signal may include a first current and a second current. The method may further include energizing, by the signal generator, a first coil of the coil array is energized by the first current in a clockwise direction and/or a counter-clockwise direction, and energizing, by the signal generator, a second coil of the coil array by the second current in a clockwise direction and/or a counter-clockwise direction.

In accordance with aspects of the disclosure, a system for dynamically configuring a secondary air-core coupled coil and exciting magnetic fields is presented. The system includes an RFID tag configured to transmit a return signal when energized, the RFID tag affixed to a surgical implement within a patient's body, a signal generator configured to generate an energizing signal for the RFID tag, and a coil antenna operably coupled to the signal generator, the antenna configured to receive a return signal transmitted by the RFID tag, the coil antenna configured to excite a magnetic field in multiple directions based on the energizing signal.

In yet another aspect of the present disclosure, the coil antenna may include a coil array including a plurality of coils. Each coil of the coil array may include a primary coil and a secondary coil.

In a further aspect of the present disclosure, the coil antenna may further include a coil tuning network configured to tune a quality factor “Q” and/or an operating frequency of the primary coil.

In yet a further aspect of the present disclosure, the tuning network may include a real part match detection network configured to detect the real part of the energizing signal, an imaginary part match detection network configured to detect the imaginary part of the energizing signal, a dynamic matching network configured to tune a quality factor “Q” and/or a first resonant frequency of the primary coil, a processor, and a memory with instructions stored thereon, which when executed by the processor cause the system to detect a real part of the energizing signal, by the real part match detection network, detect a real part of the energizing signal, by the imaginary part match detection network, determine a second resonant frequency for the primary coil, based on the detected real part and detected imaginary part of the energizing signal, and tune, by the dynamic matching network, the primary coil to the second resonant frequency, based on the determination.

In an aspect of the present disclosure, tuning network may further include a power detection network configured to detect a power level from the energizing signal.

In another aspect of the present disclosure, the instructions, when executed, may further cause the system to detect, power detection network, the power level of the energizing signal, determine a third resonant frequency for the primary coil, and tune, by the dynamic matching network, the primary coil to the third resonant frequency, based on the determination.

In yet another aspect of the present disclosure, the coil antenna may further include a termination network configured to enable or disable discrete secondary coils of the coil array.

In a further aspect of the present disclosure, the termination network may include an impedance sensor configured to sense the impedance of each of the secondary coils of the coil array, a step down transformer configured to step down the impedance of each of the secondary coils of the coil array, a dynamic capacitive bank configured to provide a plurality of loads to each of the secondary coils of the coil array via the step down transformer, a processor, and a memory with instructions stored thereon, which when executed by the processor cause the system to determine, by the impedance sensor, the impedance of the return signal, and set the dynamic capacitive bank to one of the plurality of loads based on the determination.

In yet a further aspect of the present disclosure, the secondary coil may be a configurable secondary coil, including a plurality of configurable secondary coil sections. The configurable secondary coil may have a plurality of secondary coil configurations.

In accordance with aspects of the disclosure, the coil antenna may further include a steering network configured to enable at least one of the plurality of secondary coil configurations.

In an aspect of the present disclosure, the system may include a surgical table. The coil antenna may be embedded into the surgical table.

In another aspect of the present disclosure, the coil array may include a first coil and a second coil. The energizing signal may include a first current and a second current. The first coil and the second coil may be configured to independently be energized by the first current and the second current, respectively.

In yet another aspect of the present disclosure, the first coil of the coil array may be energized with the first current in a clockwise direction and/or a counter-clockwise direction. The second coil of the coil array may be energized with the second current in a clockwise direction and/or a counter-clockwise direction.

In accordance with aspects of the disclosure, a coil antenna includes a coil array configured to receive a return signal transmitted by an RFID tag. The coil array includes a first coil configured to generate a first magnetic field and a second coil configured to generate a second magnetic field. The coil array including a plurality of coils configured to generate a magnetic flux and steer a direction of the magnetic flux and/or a magnitude of the magnetic flux based on an energizing signal from a signal generator. Each coil of the coil array includes a primary coil and a secondary coil.

In yet a further aspect of the present disclosure, the coil antenna may further include a coil tuning network configured to tune a quality factor “Q” and/or an operating frequency of each primary coil.

In an aspect of the present disclosure, the tuning network may include a real part match detection network configured to detect the real part of an energizing signal, an imaginary part match detection network configured to detect the imaginary part of the energizing signal, a dynamic matching network configured to tune a quality factor “Q” and/or a first operating frequency of the primary coil, a processor, and a memory. The memory includes instructions stored thereon, which when executed by the processor cause the coil antenna to detect a real part of the energizing signal, by the real part match detection network, detect a real part of the energizing signal, by the imaginary part match detection network, determine a second operating frequency for the primary coil, based on the detected real part and detected imaginary part of the energizing signal, and tune, by the dynamic matching network, the primary coil to the second operating frequency, based on the determination.

In another aspect of the present disclosure, the coil antenna may further include a termination network configured to enable or disable discrete secondary coils of the coil array.

In yet another aspect of the present disclosure, the termination network may include a sensor configured to sense an impedance, a voltage, and/or a current of each of the secondary coils of the coil array.

In a further aspect of the present disclosure, the secondary coil may be a configurable secondary coil, including a plurality of configurable secondary coil sections. The configurable secondary coil having a plurality of secondary coil configurations.

In accordance with aspects of the disclosure, a method for interrogation and detection of surgical implements within a patient's body, includes transmitting an energizing signal by a coil antenna operably coupled to a signal generator, the antenna configured to receive a return signal transmitted by an RFID tag, the coil antenna including a coil array, receiving a return signal, by the coil array. The coil array is configured to generate a magnetic flux and steer a direction of the magnetic flux and/or a magnitude of the magnetic flux based on the energizing signal. The method further includes detecting a real part of the energizing signal, by a real part match detection network; detecting a real part of the energizing signal, by an imaginary part match detection network; determining a second operating frequency for the primary coil, based on the detected real part and detected imaginary part of the energizing signal; and tuning, by a dynamic matching network, the primary coil to the second operating frequency, based on the determination.

In accordance with aspects of the disclosure, a system for real-time dynamically tuning an impedance match between an antenna coil and a signal generator, includes an RFID tag configured to transmit a return signal when energized, the RFID tag affixed to a surgical implement within a patient's body, a signal generator configured to generate an energizing signal for the RFID tag, a coil antenna operably coupled to the signal generator, the antenna configured to receive a return signal transmitted by the RFID tag, and a real-time tuning network configured to dynamically tune the impedance match between the signal generator and the coil antenna.

In yet a further aspect of the present disclosure, the real-time tuning network may include a tuning discriminator configured to determine a real part of the energizing signal and/or an imaginary part of the energizing signal, a phase compensation network configured to dynamically tune a phase of the matching impedance based on the determined imaginary part of the energizing signal, and a magnitude compensation network configured to dynamically tune a magnitude of the matching impedance based on the determined real part of the energizing signal. The imaginary part includes a capacitive signal, an inductive signal and/or a composite signal. The real part includes an energizing signal current and/or an energizing signal voltage.

In an aspect of the present disclosure, the real-time tuning discriminator may include an impedance transform network configured to transform the energizing signal from the signal generator for identification of the real part and the imaginary part of the energizing signal, a rectifier configured to rectify the impedance transformed signal, and a low pass filter configured to filter the rectified signal.

In another aspect of the present disclosure, the real-time tuning network may further include a power detector configured to detect the energizing signal current and/or the energizing signal voltage.

In yet another aspect of the present disclosure, the phase compensation network may include a dynamic capacitive element configured to select a frequency range for the phase of the matching impedance and a transformer configured to reduce a voltage across the dynamic capacitance element.

In a further aspect of the present disclosure, the magnitude compensation network may include a dynamic capacitive element configured to select a frequency range of the magnitude of matching impedance and a transformer configured to reduce a voltage across the dynamic capacitance element.

In yet a further aspect of the present disclosure, the system may further include a processor and a memory, including instructions stored thereon, which, when executed, cause the system to determine an imaginary part of the energizing signal and dynamically tune the phase of the matching impedance based on the determined imaginary part of the energizing signal by the phase compensation network.

In an aspect of the present disclosure, instructions when executed may further cause the system to determine a real part of the energizing signal and dynamically tune, by the magnitude compensation network, the magnitude of the matching impedance based on the determined real part of the energizing signal of the energizing signal.

In another aspect of the present disclosure, the coil antenna may include a primary coil and a secondary coil.

In yet another aspect of the present disclosure, the tuning network may be disposed between the primary coil and the signal generator. The tuning network may be configured to tune a quality factor “Q” and/or an operating frequency of the primary coil.

In a further aspect of the present disclosure, the system may further include a second tuning network electrically coupled to the secondary coil and configured to tune a quality factor “Q” and/or an operating frequency of the secondary coil.

In accordance with aspects of the disclosure, a method for real-time dynamically tuning an impedance match between an antenna coil and a signal generator, includes determining a real part of an energizing signal of the signal generator and/or an imaginary part of an energizing signal of the signal generator and dynamically tuning, by a phase compensation network, a phase of the matching impedance based on the determined imaginary part of the energizing signal.

In yet a further aspect of the present disclosure, the method may further include determining a real part of the energizing signal and dynamically tuning, by a magnitude compensation network, a magnitude of the matching impedance based on the determined real part of the energizing signal of the energizing signal.

In accordance with aspects of the disclosure, a real-time tuning network configured to dynamically tune an impedance match between a signal generator and an antenna, is presented. The real-time tuning network including a real-time tuning discriminator configured to determine a real part of an energizing signal from the signal generator and/or an imaginary part of the energizing signal, a phase compensation network configured to dynamically tune a phase of the matching impedance based on the determined imaginary part of the energizing signal, and a magnitude compensation network configured to dynamically tune a magnitude of the matching impedance based on the determined real part of the energizing signal. The imaginary part includes a capacitive signal, an inductive signal, and/or a composite signal. The real part includes an energizing signal current and/or an energizing signal voltage.

In an aspect of the present disclosure, the real-time tuning discriminator may include an impedance transform network configured to transform the energizing signal from the signal generator for identification of the real part and the imaginary part of the energizing signal, a rectifier configured to rectify the impedance transformed signal, and a low pass filter configured to filter the rectified signal.

In another aspect of the present disclosure, the real-time tuning network may further include a power detector configured to detect the energizing signal current and/or the energizing signal voltage.

In yet another aspect of the present disclosure, the phase compensation network may include a dynamic capacitive element configured to select a frequency range for the phase of the matching impedance and transformer configured to reduce a voltage across the dynamic capacitance element.

In a further aspect of the present disclosure, the magnitude compensation network may include a dynamic capacitive element configured to select a frequency range of the magnitude of matching impedance and a transformer configured to reduce a voltage across the dynamic capacitance element.

In yet a further aspect of the present disclosure, the coil antenna may include a primary coil and a secondary coil.

In an aspect of the present disclosure, the tuning network may be disposed between the primary coil and the signal generator. The tuning network is configured to tune a quality factor “Q” and/or an operating frequency of the primary coil.

In accordance with aspects of the disclosure, a system for matching an impedance between an antenna and a signal generator, includes an RFID tag configured to transmit a return signal when energized, the signal generator configured to generate an energizing signal for the RFID tag, a coil antenna operably coupled to the signal generator, the antenna configured to receive a return signal transmitted by the RFID tag, and a first transformer configured to match the impedance between the signal generator and the antenna. The RFID tag is affixed to a surgical implement within a patient's body.

In another aspect of the present disclosure, the first transformer may include a primary winding and a secondary winding. The system further comprises a capacitor disposed in parallel to the primary winding of the first transformer, the capacitor is configured to match the impedance between the signal generator and the antenna.

In yet another aspect of the present disclosure, the system may further include a matching network disposed between the first transformer and the antenna. The matching network may be configured to match the impedance between the signal generator and the antenna.

In a further aspect of the present disclosure, the matching network may include a fixed matching network and/or a dynamic matching network. The dynamic matching network is configured to dynamically match the impedance between the signal generator and the antenna based on a parameter of the energizing signal.

In yet a further aspect of the present disclosure, the parameter of the energizing signal may include a power level, a frequency, a bandwidth, a voltage, and/or a current.

In accordance with aspects of the disclosure, may further include a second transformer configured to transform an impedance match between the matching network and the antenna. The second transformer may be disposed between the matching network and the antenna.

In an aspect of the present disclosure, the second transformer may be a step up transformer.

In another aspect of the present disclosure, the system may further include a dynamic capacitive element configured to tune the impedance. The dynamic capacitive element may disposed across the primary winding of the second transformer.

In yet another aspect of the present disclosure, the system may further include a sensor configured to sense a signal indicative of a parameter of the energizing signal, a processor, and a memory. The memory includes instructions stored thereon, which when executed cause the system to sense the signal indicative of a parameter of the energizing signal, determine parameter of the energizing signal based on the sensed signal, and dynamically tune the dynamic capacitive element based on the determined parameter of the energizing signal.

In a further aspect of the present disclosure, the second transformer may include a primary winding and a secondary winding. The system further comprises a bulk capacitance disposed across the secondary winding of the second transformer.

In yet a further aspect of the present disclosure, the second transformer may include a step down transformer.

In accordance with aspects of the disclosure, a system for matching an impedance between an antenna and a signal generator, includes an RFID tag configured to transmit a return signal when energized, the signal generator configured to generate an energizing signal for the RFID tag, the antenna operably coupled to the signal generator, the antenna configured to receive a return signal transmitted by the RFID tag, a transformer configured to match the impedance between the signal generator and the antenna, and a dynamic capacitive element configured to tune the impedance, the dynamic capacitive element is disposed across the primary winding of the transformer. The RFID tag affixed to a surgical implement within a patient's body.

In an aspect of the present disclosure, the system may further include a sensor configured to sense a signal indicative of a parameter of the energizing signal, a processor, and a memory. The memory includes instructions stored thereon, which when executed cause the system to sense the signal indicative of a parameter of the energizing signal, determine parameter of the energizing signal based on the sensed signal, and dynamically tune the dynamic capacitive element based on the determined parameter of the energizing signal.

In another aspect of the present disclosure, the parameter of the energizing signal may include a power level, a frequency, a bandwidth, a voltage, and/or a current.

In yet another aspect of the present disclosure, the transformer may include a primary winding and a secondary winding. The system may further include a bulk capacitance disposed across the secondary winding of the transformer.

In a further aspect of the present disclosure, the transformer may be a step down transformer.

In yet a further aspect of the present disclosure, the system may further include a matching network disposed between the transformer and the antenna, and the matching network is configured to match the impedance between the signal generator and the antenna.

In an aspect of the present disclosure, the matching network may include a fixed matching network and/or a dynamic matching network. The dynamic matching network is configured to dynamically match the impedance between the signal generator and the antenna based on a parameter of the energizing signal.

In accordance with aspects of the disclosure, a method for tuning an impedance match between an antenna and a signal generator, the method includes sensing, by a sensor, a signal indicative of a parameter of an energizing signal, determining parameter of the energizing signal based on the sensed signal, and dynamically tuning a dynamic capacitive element based on the determined parameter of the energizing signal. The dynamic capacitive element is disposed across a primary of a transformer disposed between the signal generator and the antenna.

In yet another aspect of the present disclosure, the parameter of the energizing signal may include a power level, a frequency, a bandwidth, a voltage, and/or a current.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged or shrunk and positioned to improve drawing legibility. Further, the particular shapes of the elements as drawn are not intended to convey any information regarding the actual shape of the particular elements and have been solely selected for ease of recognition in the drawings.

Various aspects of the presently disclosed antennae, RF tags, and articles containing them are described hereinbelow with reference to the drawings.

FIG. 1 is a schematic diagram showing a surgical environment illustrating a medical provider using an interrogation and detection system to detect an object within a patient that is tagged with an RFID tag according to one illustrated aspect;

FIG. 2 is a schematic illustration of an antenna for detection of surgical implements within a patient's body in active use within a surgical site;

FIG. 3 is an image of an air-core coupled secondary reader antenna coil;

FIG. 4 is a block diagram of the air-core coupled secondary reader antenna coil of FIG. 3;

FIGS. 5A-5D are side cutaway views of the primary and secondary conductors of the air-core coupled secondary reader antenna coil of FIG. 3;

FIG. 6A is a perspective view of the air-core coupled secondary reader antenna coil of FIG. 3 with a non-staggered secondary conductor configuration;

FIG. 6B is a side view of the air-core coupled secondary reader antenna coil of FIG. 6A with the secondary conductor having two turns;

FIG. 6C is a side view of the air-core coupled secondary reader antenna coil of FIG. 6A with the secondary conductor having four turns;

FIG. 7A is a perspective view of the air-core coupled secondary reader antenna coil of FIG. 3 with a staggered secondary conductor configuration;

FIG. 7B is a top view of the air-core coupled secondary reader antenna coil of FIG. 7A with the staggered secondary conductor configuration;

FIGS. 8A and 8B are illustrations of a top view of a planar coil;

FIG. 9 is an illustration of a top view of a staggered planar coil;

FIGS. 10A and 10B are illustrations of conductors of the coils of FIGS. 8A and 9 in a non-staggered and a staggered arrangement;

FIG. 11, a graph depicting sensor output voltage vs height for a staggered coil and a non-staggered coil is shown;

FIG. 12 a graph depicting sensor output voltage vs height for a staggered secondary coil and a non-staggered secondary coil is shown;

FIG. 13 is a diagram depicting a top view of a coil array for use with the system of FIG. 1;

FIGS. 14A-14F are diagrams illustrating a direction of a magnetic field relative to a transmission line;

FIG. 15A is a perspective view of the coil array of FIG. 13, where each of the coils are excited in opposing directions;

FIG. 15B is a side view of a magnetic field of the coil array of FIG. 13, where each of the coils are excited in opposing directions;

FIG. 15C is a perspective view of a magnetic field of the coil array of FIG. 13, where each of the coils are excited in opposing directions;

FIG. 16A is a perspective view of the coil array of FIG. 13, where each of the coils are excited in the same direction;

FIG. 16B is a side view of a magnetic field of the coil array of FIG. 13, where each of the coils are excited in the same direction;

FIG. 16C is a perspective view of a magnetic field of the coil array of FIG. 13, where each of the coils are excited in the same direction;

FIGS. 17-19 are perspective views of a four element coil array for use with the system of FIG. 1, where each of the coils are excited in various directions;

FIG. 20 is a block diagram of a system for dynamically configuring a secondary coil and exciting magnetic fields in multiple directions for use with the system of FIG. 1;

FIG. 21 is a block diagram of a four element coil array for use with the system of FIG. 1;

FIG. 22 is a block diagram of a tuning network for use with the coil array of FIG. 20;

FIG. 23 is a block diagram of a termination network for use with the coil array of FIG. 20;

FIG. 24A depicts a H field vector plot when two pairs of coils are driven in phase;

FIG. 24B depicts a H field vector plot when two pairs of coils are driven in opposite phase;

FIG. 25A depicts a vector field intensity and direction for a “horizontal” current steering configuration in the X-Z plane;

FIG. 25B depicts a vector field intensity and direction for a “horizontal” current steering configuration in the X-Z plane;

FIG. 26 depicts an isometric view of H field strength in a configuration where the secondary coil is terminated to eliminate a contribution in a vertical orientation configuration;

FIG. 27 depicts a four element coil array for use with the detection system 10 of FIG. 1;

FIG. 28A-28C depict optimal Cartesian orientations in two dimensions for each discrete secondary coil configuration of the coil array of FIG. 27;

FIG. 29 is an isometric view of the coil array of FIG. 27;

FIGS. 30-32 are depictions of the primary coil current directions, configurable secondary coil current directions, steering network configuration, and optimal RFID tag orientation directions for three possible configurations of the coil array of FIG. 29;

FIGS. 33-36, the system may include a configurable air-core coupled secondary coil;

FIG. 37 depicts an example two element coil array for use with the detection system 10 of FIG. 1;

FIG. 38 depicts a high level block diagram for a system for real-time dynamic tuning of an antenna coil to a generator of the interrogation and detection system of FIG. 1;

FIG. 39 is a table depicting the permittivity and conductivity of various tissue types;

FIG. 40 is a schematic for a tuning discriminator of the system of FIG. 38;

FIG. 41 is a schematic of a phase compensation network and magnitude compensation network of the system of FIG. 38;

FIG. 42 an integrated Q factor sensor for use with the system of FIG. 38;

FIG. 43 is a graph illustrating the linear output of the “Q”-factor sensor of FIG. 41;

FIG. 44 is a flow diagram for a method for real-time dynamically tuning an impedance match between an antenna coil and a generator;

FIG. 45 is a diagram of a transformer for matching an output impedance of a signal generator to an antenna of the interrogation and detection system of FIG. 1;

FIG. 46 is a diagram of a loop antenna of the interrogation and detection system of FIG. 1;

FIG. 47 is schematic of a transformer model which includes including parasitics;

FIG. 48 is a schematic of a transformer model, including reflecting a parasitic capacitance though the transformer;

FIG. 49 is a diagram of a transformer matching system for use with the interrogation and detection system of FIG. 1;

FIG. 50 is a diagram of a transformer matching system that incorporates a dynamic capacitive element for a parallel capacitance, for use with the interrogation and detection system of FIG. 1; and

FIG. 51 is a diagram of a system for matching an impedance of a generator to an antenna.

### DETAILED DESCRIPTION

In the following description, certain specific details are set forth in order to provide a thorough understanding of disclosed aspects. However, one skilled in the relevant art will recognize that aspects may be practiced without one or more of these specific details or with other methods, components, materials, etc. In other instances, well-known structures associated with transmitters, receivers, or transceivers have not been shown or described in detail to avoid unnecessarily obscuring descriptions of the aspects.

Reference throughout this specification to “one aspect” or “an aspect” means that a particular feature, structure, or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics may be combined in any suitable manner in one or more aspects.

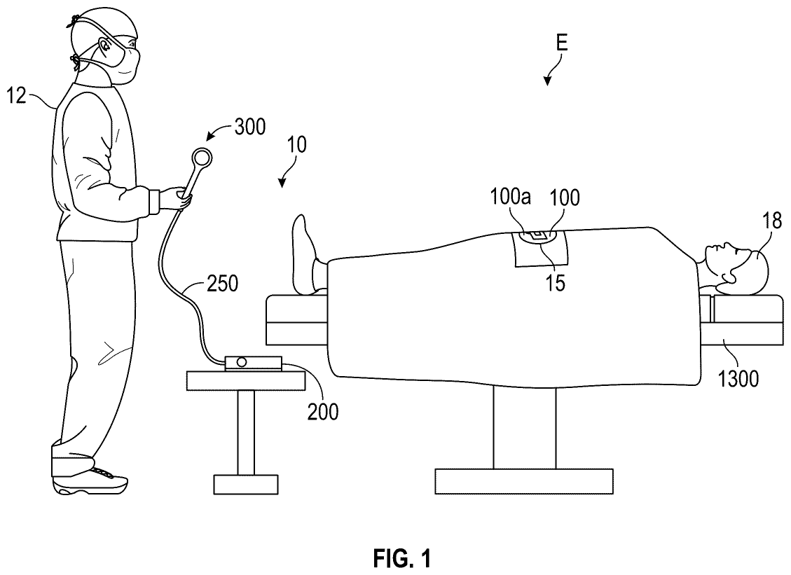

FIG. 1 depicts a surgical environment “E” in which a medical provider 12 operates an interrogation and detection system 10 for detection of radio-frequency identification (RFID) tags to ascertain the presence or absence of items, implements or objects 100a in a patient 18. The interrogation and detection system 10 may include a signal generator 200 and an antenna 300 coupled to the signal generator 200 by one or more communication paths, for example, coaxial cable 250. In one aspect of the interrogation and detection system 10, the antenna 300 may take the form of a hand-held wand 300a.

The object 100a may take a variety of forms, for example, instruments, accessories, and/or disposable objects useful in performing surgical procedures. For instance, the object 100a may take the form of scalpels, scissors, forceps, hemostats, and/or clamps. Also, for example, the objects 100a may take the form of surgical sponges, gauze, and/or padding. The object 100a is tagged, carrying, attached, or otherwise coupled to an RFID tag 100. Aspects of the interrogation and detection system 10 disclosed herein are particularly suited to operate with one or more RFID tags 100, which are not accurately tuned to a chosen or selected resonant frequency. Consequently, the RFID tags 100 do not require high manufacturing tolerances or expensive materials and thus may be inexpensive to manufacture.

In use, the medical provider 12 may position the wand 300a approximate the patient 18 in order to detect the presence or absence of the one or more RFID tags 100 and hence an object 100a. The medical provider 12 may, in some aspects, move the wand 300a along and/or across the body of the patient 18. For a detailed description of an exemplary interrogation and detection system, reference may be made to commonly owned U.S. Patent Application Publication No. 2004/0250819 to Blair et al., titled “Apparatus and Method for Detecting Objects Using Tags And Wideband Detection Device,” filed Mar. 29, 2004, the entire contents of which is hereby incorporated by reference herein.

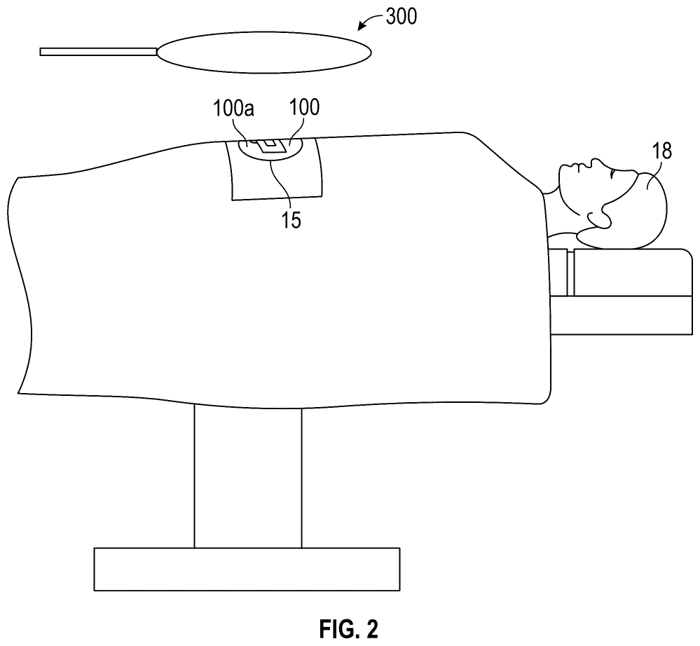

Referring now to FIG. 2, interrogation and detection system 10, for detection of surgical implements (e.g., object 100a) within a patient's body, includes a signal generator 200 to provide an energizing signal for one or more RFID tags 100 (FIG. 1) affixed to an object 100a (FIG. 1). Each RFID tag 100 is configured to transmit a return signal when energized, such that an antenna 300 can detect the return signal and confirm the presence of objects 100a within the body of patient 18. The antenna 300 is operably coupled to the signal generator 200 via a communication cable 250. Where the communication cable 250 may be of variable length to provide greater range of motion to the clinician handling the antenna 300. In aspects, the antenna may include, for example, a wireless hand-held unit which is battery operated.

In one aspect of interrogation and detection system 10, the antenna 300 is an antenna 300 configured to be waved over the surgical site 15, e.g., over the body of patient 18. For example, the antenna 300 may be held over the body of the patient 18 at the height of about four or about five inches while attempting to detect an RFID tag so that the user may detect confirm the presence of objects 100a within the body of patient 18.

The term “read range,” as used in this disclosure, includes the distance from an antenna 300 (e.g., a reader coil) of an interrogation and detection system 10 and the geometric center of the RFID tag 100 (FIG. 1). The term “orientation,” as used in this disclosure, includes the angle of incidence between the primary coil plane of the antenna 300 and the primary coil plane of the tag antenna or coil. Generally, for optimal orientation, the primary reader coil plane and the primary tag coil plane are parallel. Non-optimal orientation implies the primary reader coil plane, and the primary tag coil plane are not parallel, with the most challenging orientation being where the primary reader coil plane and the primary tag coil plane are orthogonal in space.

For a hand-held accessory, the physical size is important. The accessory needs to be constructed using a form factor that is conducive to use in the intended application. For typical RFID applications, where communication between an interrogation and detection system 10 and an RFID tag 100 (FIG. 1) is accomplished via coupled magnetic fields, the read range and sensitivity to coil orientation is a direct function of the coil areas and orientation. In other words, a larger coil will perform better in terms of the range where communication is possible, as well as the relative orientation between the reader antenna 300 and the RFID tag coil. In the targeted application (detection and identification of tagged surgical items), the acceptable physical sizes of both the reader coil and the tag coil are smaller than those that would provide the optimal read range performance and reduced sensitivity to orientation. In the case of the RFID tag 100 (FIG. 1), the acceptable physical size of the tracked tag component may be much smaller than ideal. In order to achieve an acceptable user experience in terms of both performance and physical form factors, novel methods of maximizing performance must be employed.

One of the limiting factors in terms of interrogation and detection system 10 performance in the field of RFID relates to the optimal loading conditions an antenna coil may present to signal generator 200 (FIG. 1). Mismatches or changes in the interface between the signal generator 200 (FIG. 1) and the antenna 300 result in a non-optimal transmission of the RF power from the signal generator 200 to the antenna 300. These mismatches can cause the RF energy to be reflected back into the signal generator 200 instead of propagating into the antenna 300 and then from the antenna 300 into the wireless communications channel with the RFID tag 100 (FIG. 1). Conventional generators 200 of RFID systems are generally designed to interface to a fixed static load with primarily only a real impedance component (Z=Re{ }). However, tissue salinity will affect the quality factor “Q” and the impedance of the antenna 300 (e.g., the reader coil). The quality factor “Q” of an antenna directly influences the bandwidth of the antenna as well as the gain of the antenna.

Frequently, generators 200 of RFID systems are intended to interface to a 50-ohm characteristic impedance antenna. This convention allows developers to abstract the development of reader solutions from the coil or antenna solutions. A typical antenna coil presents a predominantly reactive (inductive, Z=Im{ }) load impedance at the coil terminals. A matching network is used to interface a predominantly real generator (e.g., reader) output impedance to a predominantly reactive coil input impedance. This is usually done in two steps, with one aspect of the matching network being used to accommodate the real part of the target load and another aspect of the matching network canceling out the reactive part of the coil load. The end result is an optimal transmission of the RF energy to and from the generator and antenna 300 elements. In practical applications, the acceptable range of the complex part of the load impedance in the antenna coil is limited due to the topologies and component values required to construct an effective matching network. As a rule of thumb, the coil inductance may nominally be between about 0.5 uH and about 2.5 uH for practical matching networks. Coils with reactive impedances outside of this range may be difficult to match using conventional methods and susceptible to component tolerance and drift.

A coil's inductive impedance component is largely a function of two main characteristics. The coil area, and the number of turns in the coil. Magnetic field vector magnitude, and thus the ability to energize and communicate with a passive tag for a given coil current is a direct function of both the coil area and the number of turns in the coil. In other words, as the coil area and/or the number of turns in the coil increase, the read range increases. Unfortunately, for coils intended to be used in long-range wireless magnetic-based communications, the inductance due to the coil area quickly eliminates the option of adding turns to the coil due to the increase in the inductive component of the impedance. Inductance is based on the square of the number of turns. Ultimately, the ability to maximize the performance of an antenna coil directly through physical characteristics is limited by the amount of inductive load the coil presents to the matching network. A method that allows for increasing the coil inductance while maintaining a practical matching load impedance is of interest. The disclosed technologies leverage non-traditional methods that enable either an increase in coil area or an increase in the number of coil turns may achieve improved read range and/or sensitivity to antenna 300/RFID tag 100 orientation.

In addition to the limitations relating to the allowed range of reactive impedance described above, is a limitation in the ability to tune an antenna to operate at resonance. Resonance in this context is a scenario where an inductive coil is loaded with an equal and opposite capacitive load (either in series or in parallel). The resultant circuit in the ideal case is capable of sustaining an oscillation at the resonance frequency indefinitely. According to the present disclosure, resonant circuit techniques are used to improve coil-type antenna performance by providing a dimension of performance gain, frequently referred to as a quality factor (Q). The quality factor “Q” of an antenna is a way of describing the ratio of the reactive part of the impedance to the real part of the impedance (Im{ }/Re{ }). The reactive part of impedance is theoretically a lossless impedance, and the real part of the impedance is theoretically totally lossy. In other words, the energy imparted to a resonant coil antenna oscillates with an efficiency that is described by the ratio of the lossless impedance to the lossy impedance. As the inductive and capacitive components are tuned to be equal to each other at the target operating frequency, the quality factor increases, and the ratio of energy in the lossless components of the impedance is increased when compared to the energy dissipated in the lossy part of the impedance. Maximizing the quality factor “Q” is, in essence, a way of realizing free “gain” in a wireless communications channel. There are limitations in the allowed magnitude of the quality factor in conventional communications channels, but the limitation we are specifically addressing with this solution relates to the ability to match a generator output channel to an antenna 300 a resonant coil input.

For ideal parallel resonant circuits, the magnitude of the impedance approaches infinity (open circuit), and the voltage across the coil element is maximized. For ideal serial resonant circuits, the impedance approaches zero “0” (short circuit), and the current through the coil element is maximized. Reader technologies are not designed to drive open or short circuit loads, so we are forced to de-tune (tune away from resonance) the resonant properties in order to match a reader to a coil from a practical perspective. This results in a reduction of the free gain that may be realized via the quality factor “Q.” Furthermore, changes in the dielectric properties of the space around the antenna coils will shift the resonant frequency of the coil or degrade the coil performance due to an increase in the real part of the coil impedance.

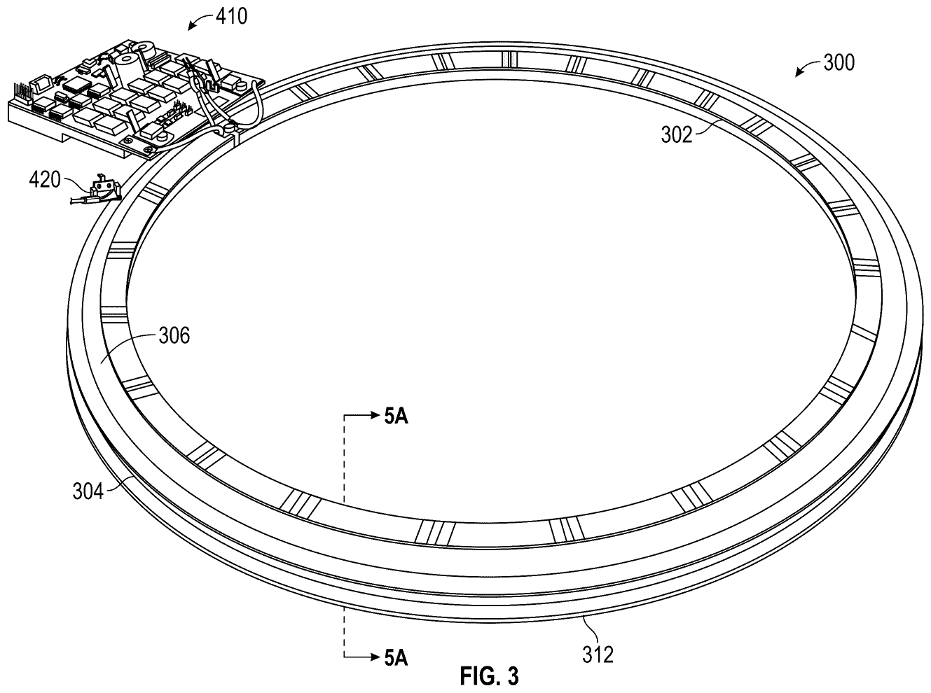

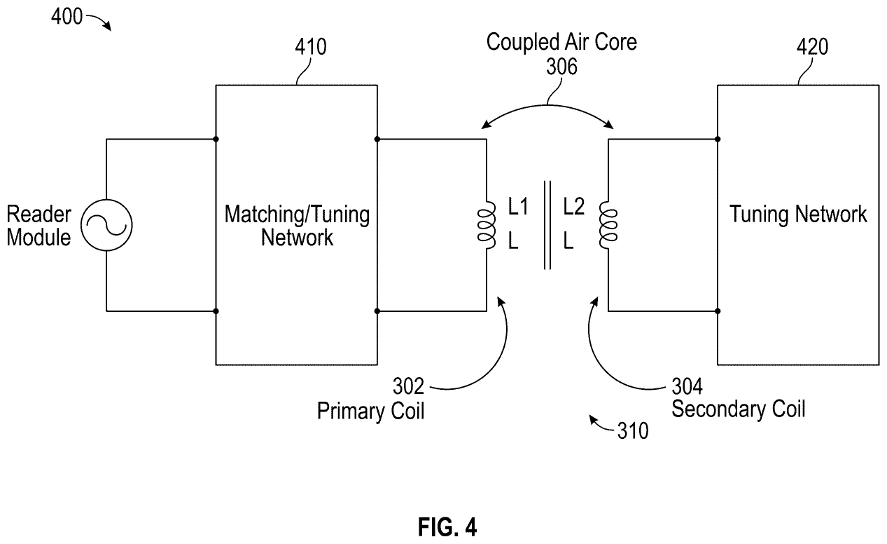

With reference to FIGS. 3 and 4, an air-core coupled secondary reader antenna coil 300 is shown, in accordance with the disclosure. The air-core coupled secondary reader antenna coil 300 generally includes a transformer 310, a primary matching network 410, and a secondary matching network 420. As used herein, the term “air-core” may include any non-magnetic insulating material, such as polymers, and/or air.

The transformer 310 generally includes a primary coil 302, a secondary coil 304, and a core 306 made of a non-magnetic insulating material (e.g., an air-core and/or a polymer). Air has a dielectric constant of one “1” and a loss tangent of about zero “0.” It is contemplated that other materials may be used for the core that have sufficiently low loss tangents at and/or around the frequency of operation of the antenna 300 (e.g., polyimide, Polytetrafluoroethylene “PTFE,” etc.). The primary coil 302 and secondary coil 304 may be mounted to an insulating material. The first matching network 410 is configured to match an input impedance of the primary coil 302 to an output impedance of the generator 200 (FIG. 1). The second matching network 420 is configured to match an input impedance of the secondary coil 304 to an output impedance of the primary coil 302 in conjunction with the load presented by the patient or the environmental load.

Each of the primary coil 302 and the secondary coil 304 include one or more turns. In aspects, the primary coil 302 and the secondary coil 304 may have the same or a different number of turns than each other. For example, the primary coil 302 may have one turn, and the secondary coil 304 may have two turns for a “turns ratio” of 1:1. A “turns ratio” is the ratio of a transformer's primary and secondary windings with respect to each other. The primary coil 302 and/or the secondary coil 304 may be but is not limited to, any suitable shape, for example, spiral, square, elliptical, and/or circular. In aspects, primary coil 302 and/or the secondary coil 304 may have any suitable cross-section. For example, the cross-section of the primary coil 302 and/or the secondary coil 304 may be, but is not limited to, coaxial, planar, “C” shaped, and/or tube-shaped. The primary coil 302 and the secondary coil 304 may be any suitable width.

The air-core 306 is configured to provide a path for a magnetic field to flow around to induce a voltage between the primary coil 302 and the secondary coil 304. By adding an air-core 306 coupled secondary coil 304 to a traditional coil antenna, the challenges associated with both of the points discussed above are reduced. It is possible to incorporate several turns (two or more) on the secondary coil 304 while maintaining a reasonable matching impedance as presented at the primary coil 302 input terminals. This is due to the construction of a relatively weakly coupled transformer 310 with “turns ratio” 1:N, where “N” is the number of turns on the secondary coil 304. The transformer 310 acts as an impedance transformer, essentially stepping down the load (reactance) by a ratio of 1/N². The net result is that the inductance measured at the primary coil 302 terminals increases at a rate less than N², allowing for secondary coil 304 inductance values in excess of about 2.5 uH while maintaining primary coil 302 reactance components that can be reasonably matched from a practical perspective. Additionally, the secondary coil 304 can be tuned to a resonance closer to the target frequency of operation. As the load reflects through the air-core coupled transformer 310, it is reduced (parallel resonance), and the parasitic impedances that are inherent to the circuit shift the resonance as seen by the primary coil 302, further allowing a practical tuning situation.

The primary matching network 410 is configured to match the impedance between the primary coil 302 and the signal generator 200 to minimize mismatches that can cause the RF energy to be reflected back into the signal generator 200 instead of propagating into the primary coil 302. The secondary matching network 420 is configured to match the impedance between the secondary coil 304 and the primary coil 302, and between the secondary coil 304 and the tag coil (not shown) to minimize mismatches that can cause the RF energy to be reflected back into the signal generator 200 instead of propagating into the tag coil.

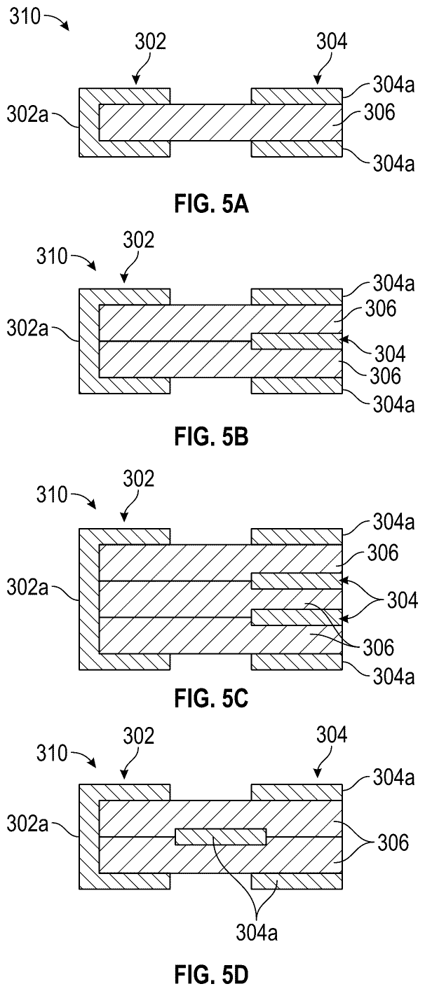

Referring to FIGS. 5A-5D side cutaway views of the primary and secondary conductors of the air-core coupled secondary reader antenna coil of FIG. 3 are shown. FIG. 5A illustrates transformer 310 with a “turns ratio” of N=2 in a non-staggered configuration. The primary coil 302 includes a “C” shaped conductor 302a. The secondary coil 304 includes two planar conductors, 304a. The air-core 306 is disposed between the primary coil 302 and the secondary coil 304.

FIG. 5B illustrates transformer 310 with a “turns ratio” of N=3 in a non-staggered configuration. The primary coil 302 includes a “C” shaped conductor 302a. The secondary coil 304 includes three planar conductors 304a. The air-core 306 is disposed between the primary coil 302 and the secondary coil 304.

FIG. 5C illustrates transformer 310 with a “turns ratio” of N=4 in a non-staggered configuration. The primary coil 302 includes a “C” shaped conductor 302a. The secondary coil 304 includes four planar conductors 304a. The air-core 306 is disposed between the primary coil 302 and the secondary coil 304.

FIG. 5D illustrates transformer 310 with a “turns ratio” of N=3 in a staggered configuration. The staggered configuration reduces parasitic capacitances which can lead to a low self-resonant frequency. The primary coil 302 includes a “C” shaped conductor 302a. The secondary coil 304 includes three planar conductors 304a. The air-core 306 is disposed between the primary coil 302 and the secondary coil 304. It is contemplated that any suitable number of turns may be used by the transformer 310.

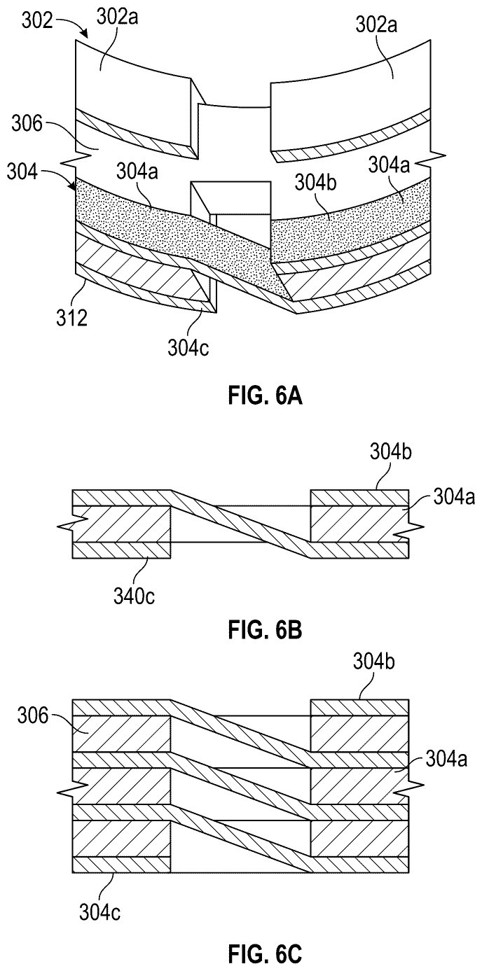

Referring to FIG. 6A, a perspective view of the air-core coupled secondary reader antenna coil 300 of FIG. 3 with a non-staggered secondary conductor configuration is shown. The primary coil 302 is shown having one turn, and the secondary coil 304 is shown having two turns. The two terminals of the secondary coil 304, e.g., “secondary coil+” 304b and “secondary coil−” 304C are configured for electrical communication with the second matching network 420 (FIG. 2). FIG. 6B shows a side view of the air-core coupled secondary reader antenna coil 300 of FIG. 6A where the secondary coil 304 has two turns to the primary coil's 302 one turn (a “turns ratio” of N=2). FIG. 6C shows a side view of the air-core coupled secondary reader antenna coil 300 of FIG. 6A with the secondary conductor having four turns (a “turns ratio” of N=4).

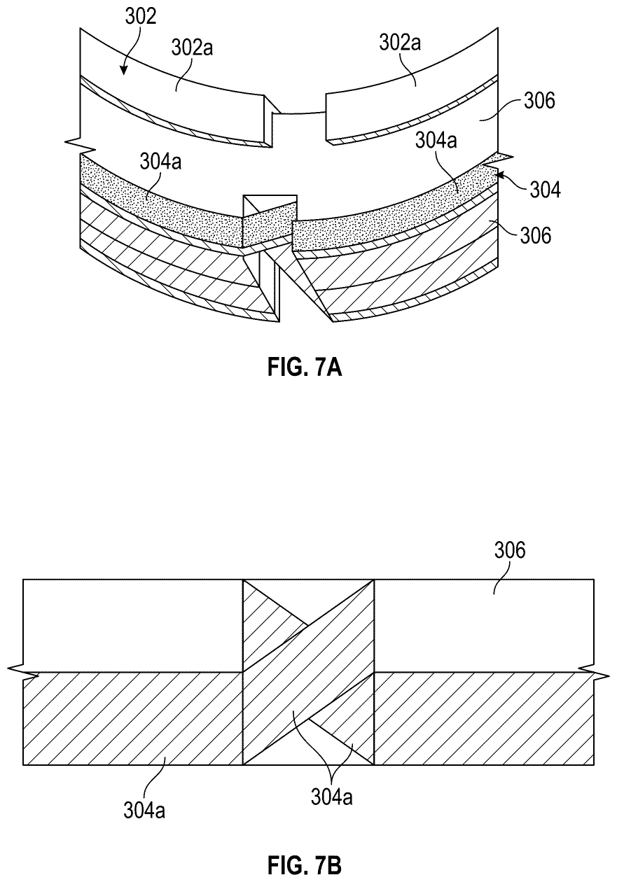

FIGS. 7A and 7B are views of the air-core coupled secondary reader antenna coil 300 of FIG. 3 with a staggered secondary coil conductor 304b configuration to reduce low self-resonant frequencies due to parasitic capacitances.

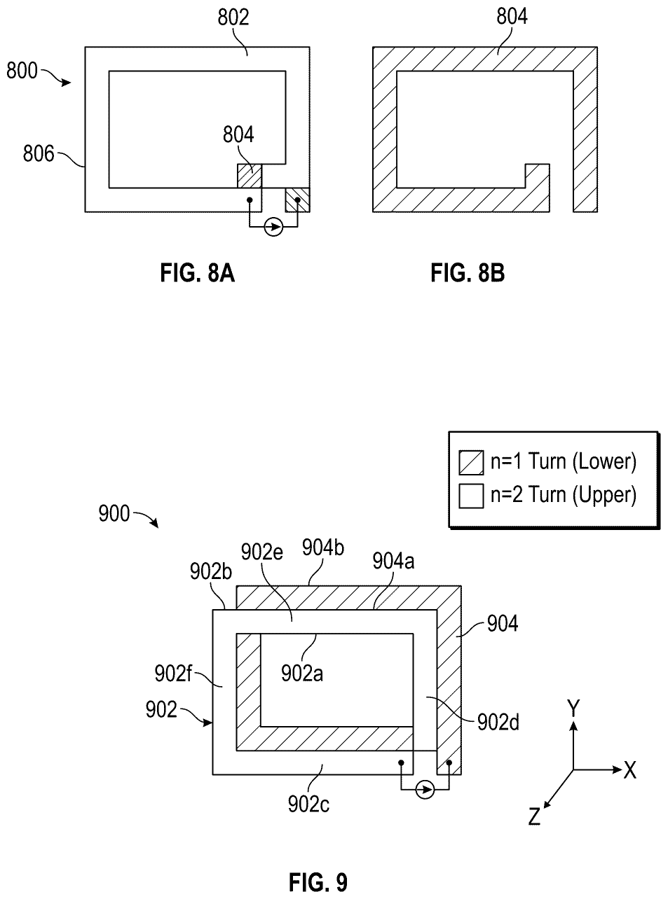

Referring to FIGS. 8A and 8B, a top view of a non-staggered coil 800, which may be used as the primary coil 302 and/or the secondary coil 304 of a reader antenna 300 (FIG. 3), in accordance with the present disclosure, is shown. A first turn conductor 802 of non-staggered coil 800 is directly located over a second turn conductor 804 of non-staggered coil 800, with a core 806 (e.g., air or another dielectric material suitable as an insulator) disposed between the conductors of the first and second turn conductors 802, 804.

Large multi-turn magnetic field coil antennas have inherently low self-resonant frequencies due to parasitic capacitances. When designing a near field radio frequency identification reader antenna, it is desirable to use a coil design that is primarily inductive in order to ensure that the current circulating through the coil conducts through the entire coil. Doing so results in maximized field strengths for a given coil current. As the parasitic capacitance increases, the currents in the coil begin to return to the source through undesirable paths, thus reducing the effectiveness of the coil. Adding to the challenge is the competing objective of designing for high coil factor “Q” (the ratio of the reactive impedance to the real impedance). A high factor “Q” coil is a coil where the ohmic real part of the resistance is minimized while the coil inductance is maximized. In aspects, increasing quality factor “Q” may be performed by increasing the conductor thickness (to compensate for skin-depth) and/or conductor surface area (or width in the case of a flat conductor). However, the parasitic capacitance between two conductors is a function of the conductor area as well as the distance between the conductors. As such, increasing the width of the conductor results in a corresponding decrease in the self-resonant frequency. For an inductive coil, increasing the separation between adjacent coil windings reaches a point of diminishing returns as the coupling efficiency between the individual coils reduces when the distance between the coils is increased.

Referring to FIG. 9 a top view of a staggered coil 900, which may be used as the primary coil 302 and/or the secondary coil 304 of a reader antenna 300 (FIG. 3), in accordance with the present disclosure, is shown. As described in detail below, by staggering the conductors of the staggered coil 900, the parasitic capacitance between the traces of the turns of the staggered coil 900 may be reduced.

A first turn conductor 902 of staggered coil 900 is located in a parallel relation and in an offset manner to the second turn conductor 904 of staggered coil 900, and in vertical and/or horizontal relation to one another in an X, Y, Z coordinate system. The first turn conductor 902 and second turn conductor 904 have a core 906 between them. The core 906 may be air or other dielectric material suitable as an insulator. Although two turns are shown, any suitable number of turns (e.g., conductive layers) may be used by offsetting the additional turns in vertical and/or horizontal relation to the previous turn conductor. For example, the staggered coil 900 may include three turns, where the third turn is offset from the second turn conductor 904 in a similar manner to how the second turn conductor 904 is offset from the first turn conductor 902.

The first turn conductor 902 includes an inner edge 902a and an outer edge 902b. The second turn conductor 904 includes an inner edge 904a and an outer edge 904b. The first turn conductor 902 may overlap the second turn conductor 904 of the staggered coil 900, where a substantial portion (e.g., about >50%) of the second turn conductor 904 is not overlapped by the first turn conductor 902.

The first turn conductor 902 and/or the second turn conductor 904 may be but are not limited to, any suitable shape, for example, spiral, square, elliptical, and/or circular. In aspects, first turn conductor 902 and/or the second turn conductor 904 may have any suitable cross-section. For example, the cross-section of the first turn conductor 902 and/or the second turn conductor 904 may be, but is not limited to, coaxial, planar, “C” shaped, and/or tube-shaped. The first turn conductor 902 and/or the second turn conductor 904 may be any suitable width (for example, approximately 1 cm wide copper). It is contemplated that the first turn conductor 902 and/or the second turn conductor 904 are monolithic, or each turn of the coil may be a single piece, electrically connected to the next turn.

This staggered coil 900 configuration combines optimized conductor width and an optimized coil-to-coil configuration with a staggered individual coil element design. In aspects, this staggered coil 900 configuration may be packaged into a thin mattress-like coil array intended for use as a scanning accessory in the real-time detection of tagged surgical items in an operating room environment. The staggered coil 900 configuration has advantages over other implementations, such as a traditional planar coil due to the preservation of the coil area, which is a dominant characteristic of the coil performance in terms of field strength. Furthermore, the staggered configuration has the added advantage of improving radiolucency when compared to a traditional coaxial coil design due to the reduced number of coexistent x-y plane conductive layers in multi-turn coils. The staggered coil 900 configuration is effective in applications where a single coil is driven directly, as well as applications where an air-core coupled secondary is utilized. For example, in a staggered coil 900 configuration, the reduction in parasitic capacitance and subsequent increase in self-resonant frequency may be highly beneficial, where maximizing coil inductance or improving coil efficiency are advantageous.

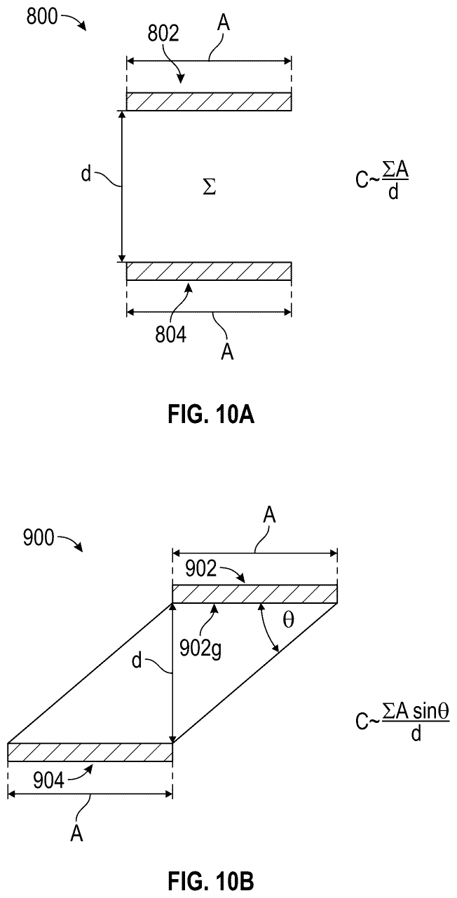

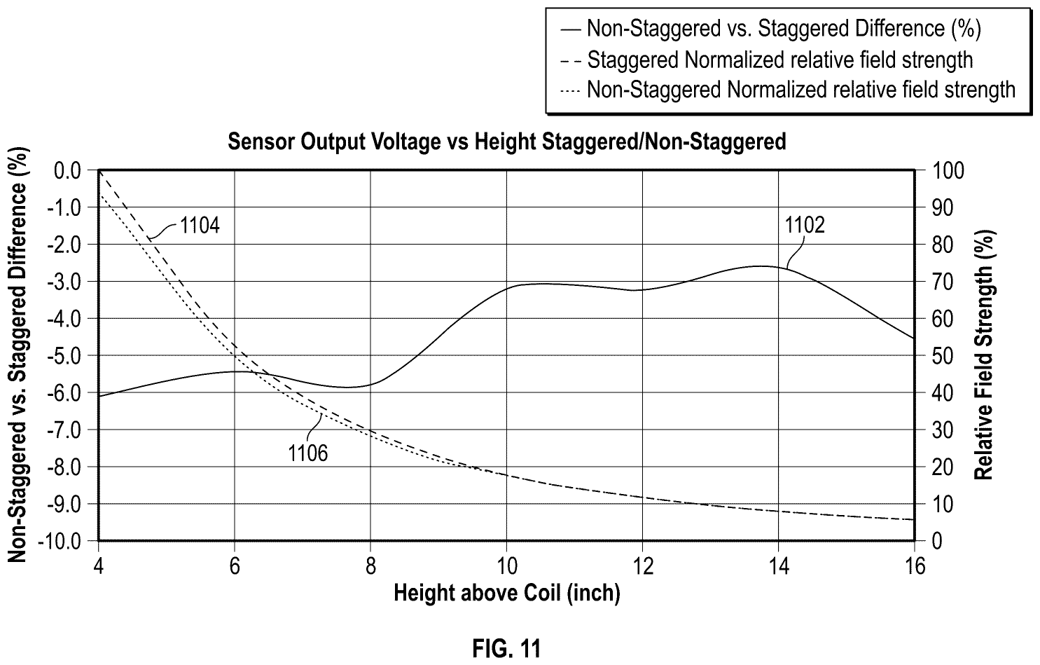

Referring to FIG. 10A and 10B, illustrations of conductors of the coils of FIGS. 8A and 9 in a non-staggered and a staggered arrangement are shown. The capacitance of two parallel-coupled conductors, such as the non-staggered configuration, is (E*A)/d, where “E” is the dielectric constant, where “d” is the distance between conductors, and “A” is the width of the conductor. By staggering the conductors, the capacitance is approximately (E*A*sin of theta)/d, where theta is the angle between the bottom surface 902g of the first turn conductor 902 and the edge of the second turn conductor 904 that is closest to the first turn conductor 902.

Referring to FIG. 11, a graph depicting sensor output voltage vs. height for a single driven staggered coil and a single driven non-staggered coil is shown. The graph also depicts an example relative field strength gain associated with staggered vs. non-staggered construction, both having a single driven coil.

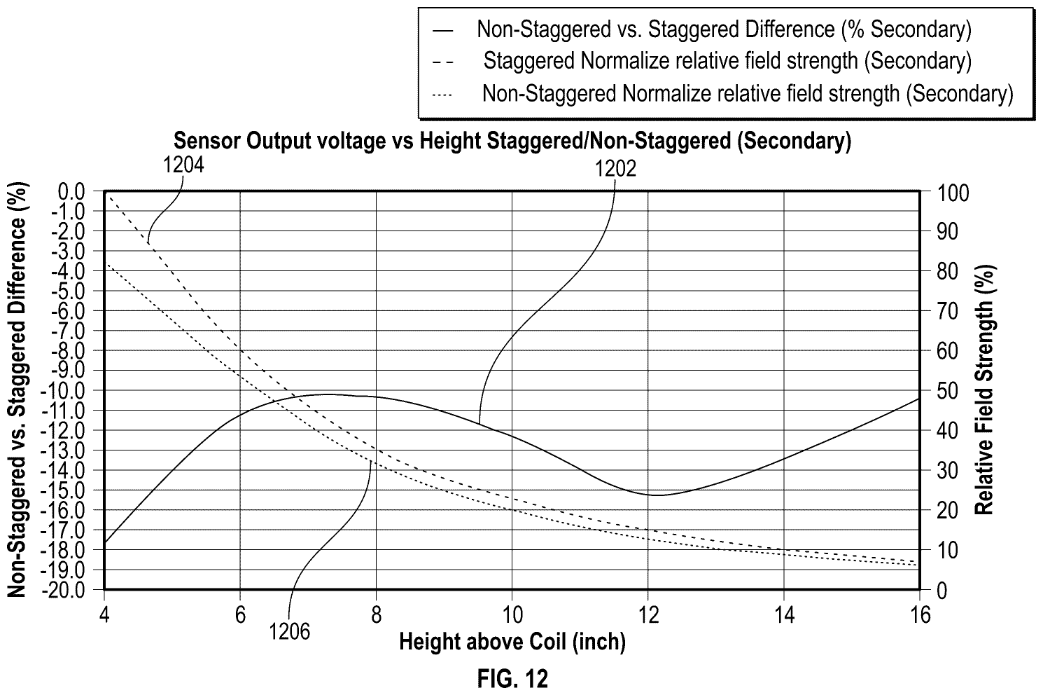

Referring to FIG. 12 a graph depicting sensor output voltage vs. height for a staggered secondary coil and a non-staggered secondary coil is shown. The graph also depicts an example of relative field strength gains associated with staggered vs. non-staggered construction, both using a secondary coupled coil.

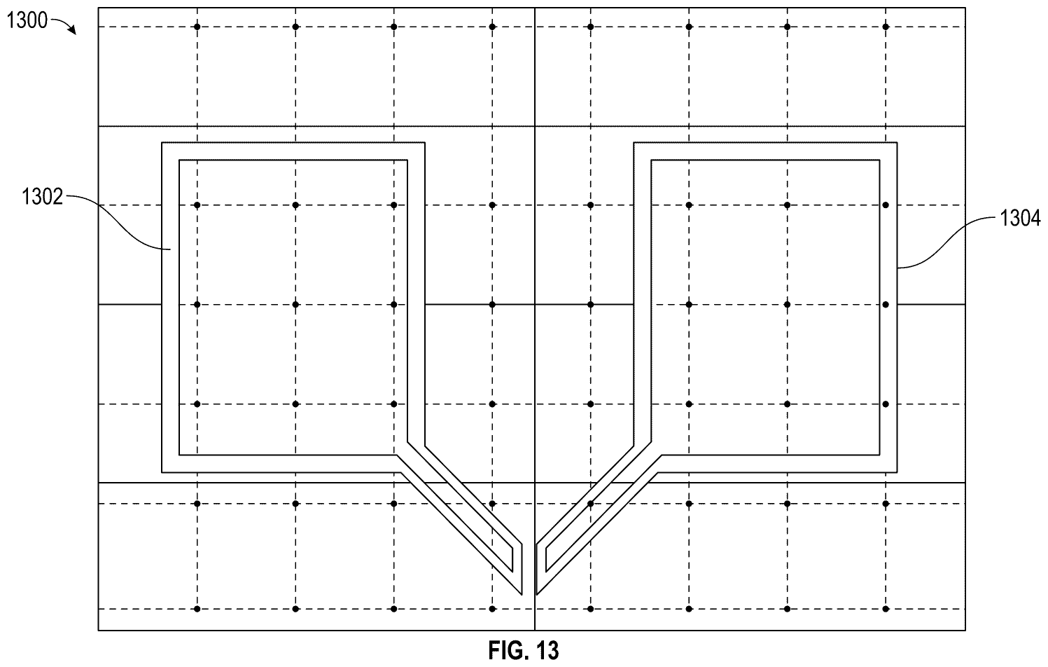

Referring to FIG. 13, a top view of a coil array 1300, which may be used as the antenna 300 (FIG. 3) of the detection system 10 (FIG. 1), in accordance with the present disclosure, is shown. Generally, a coil array includes two or more coils, for example, a first coil 1302 and a second coil 1304. The individual coils 1302, 1304 in the array are configured to be independently energizable, enabling the steering of a magnetic flux “B” of each of the coils independently. It is contemplated that each of the individual coils 1302, 1304 may include any number of turns. For example, each of the individual coils 1302, 1304 may include two turns each.

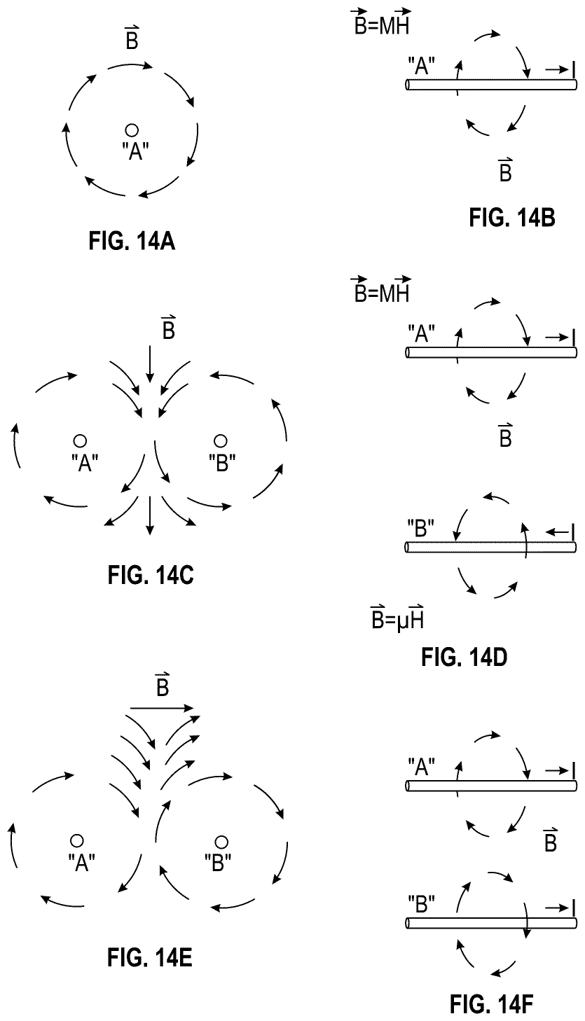

Referring to FIGS. 14A-14F are diagrams illustrating a direction of a magnetic field relative to a current of a transmission line. The magnetic field intensity “H” can be visualized by using the “right-hand-rule” (FIGS. 14A and 14B). The magnetic field intensity “H” vector directions curl around the line current “I,” forming a closed-loop (FIG. 14A). Magnetic flux density “B” is a measurement of the total magnetic field which passes through a given area and can be described by the equation B=uH. The magnetic flux density “B” that results from a line current “I” is directional in nature. In circumstances where the line current is an element of a closed-loop antenna of arbitrary geometry, the magnetic flux density “B” at a given point in space is equal to the superimposed fields from each individual line current “I” element (FIGS. 14C-14F). As such, for a fixed position coil, or a coil array 1300 (FIG. 13) that utilizes a static configuration for current direction, the vector direction of the magnetic field intensity “H” around the coil or array is fixed.

For near-field RFID communications systems, the reader antenna coil (e.g., antenna 300 of FIG. 1 or coil array 1300) and the tag antenna coil of RFID tag 100 (FIG. 1) are linked via a magnetic flux density “B” sourced by current flowing through the antenna 300 (e.g., a reader coil) (FIG. 1). The tag coil captures this flux and uses it to both energize the local tag electronics as well as for communicating information back to the antenna 300 (FIG. 1). The amount of magnetic flux density “B” captured by the tag determines the amount of energy available for the tag to perform its intended function. In the optimal orientation, where the tag coil is parallel to and coaxial with the antenna 300 (FIG. 1), the amount of magnetic flux density “B” captured is optimized. As the tag coil is moved away from this optimal orientation, the amount of magnetic flux “B” captured decreases. Once the amount of magnetic flux density “B” captured by the tag decreases to the point that sufficient energy is not captured to achieve the required activation energy for the tag electronics, the reader-tag communications channel is lost.

For scenarios where the tag is able to be oriented in any direction over a fixed reader coil/reader coil array, every point in space above the coil/array will have a magnetic field “H” vector that is optimal to a specific RFID tag 100 (FIG. 1) orientation and at the same time, non-existent (orthogonal) to another tag orientation at the same location. This means that a RFID tag 100 (FIG. 1) may be discoverable and provide information if oriented in one direction, and the RFID tag 100 (FIG. 1) may not be discoverable and provide information in a different orientation at the same location. For a hand-held reader coil (e.g., antenna 300 of FIG. 1), this issue may be overcome by manipulating the angle of incidence from the reader coil manually by changing the reader coil orientation. For a fixed array that is located, for example, under a patient (e.g., coil array 1300 of FIG. 1), the antenna orientation is fixed. This solution solves this problem by changing the magnetic field “H” vector direction via current steering in the coil array 1300. By scanning through various current/coil element configurations, the magnetic field vector direction can be changed such that it is able to energize and communicate with tags in otherwise non-optimal orientations.

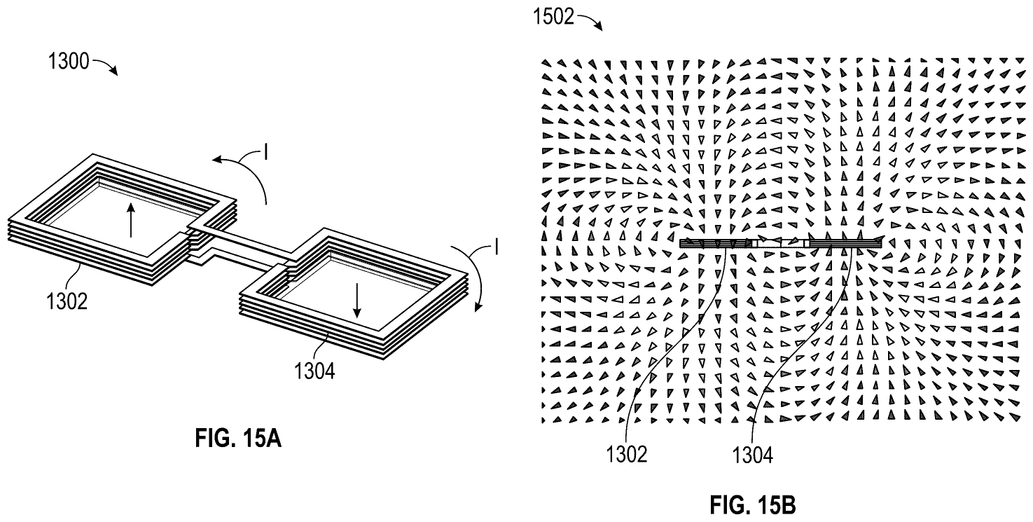

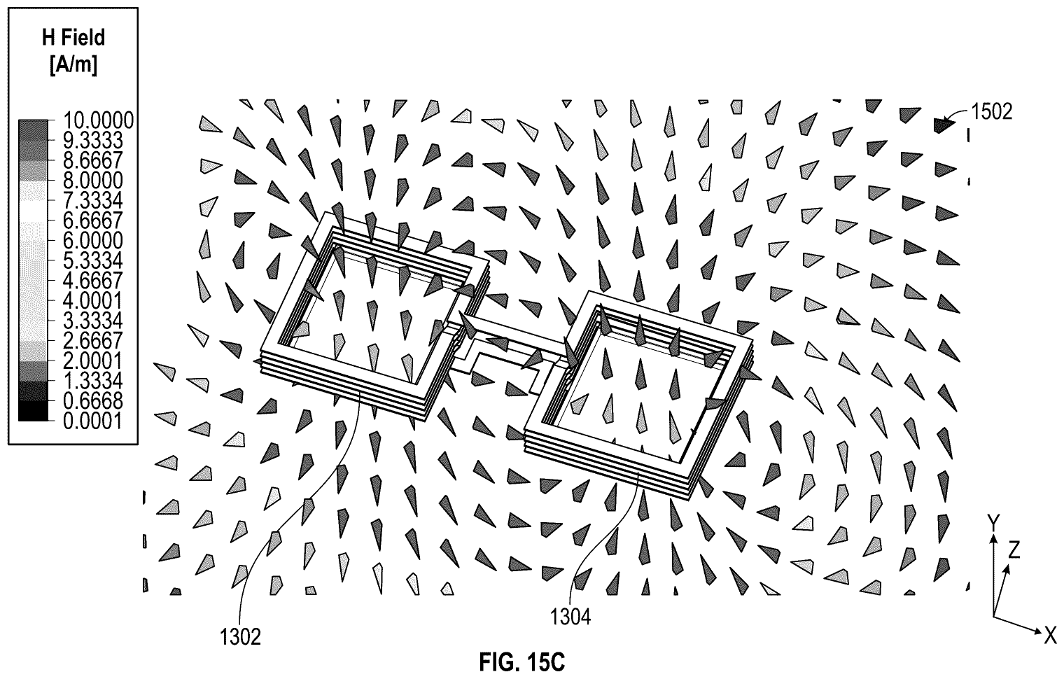

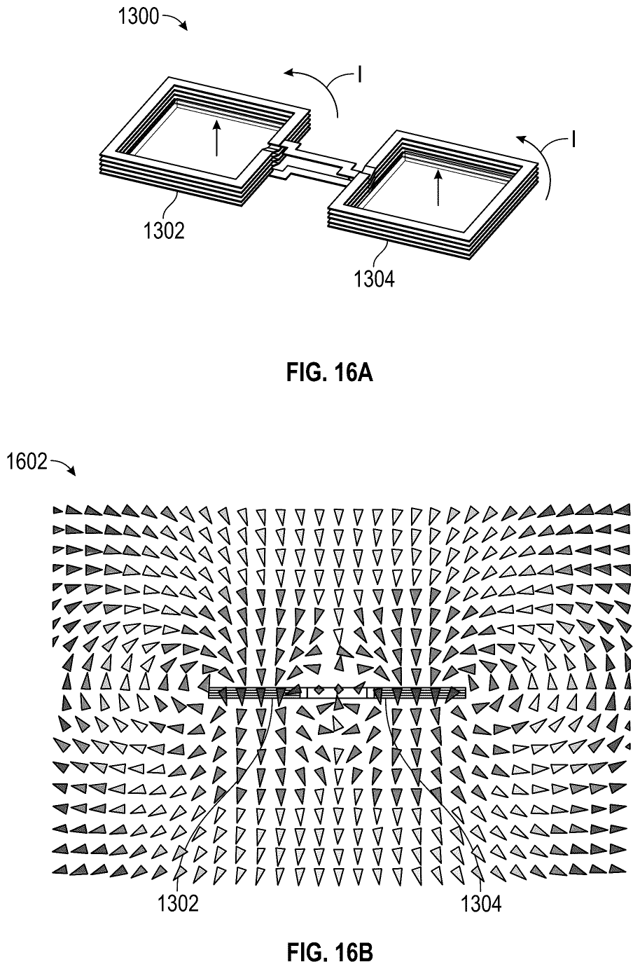

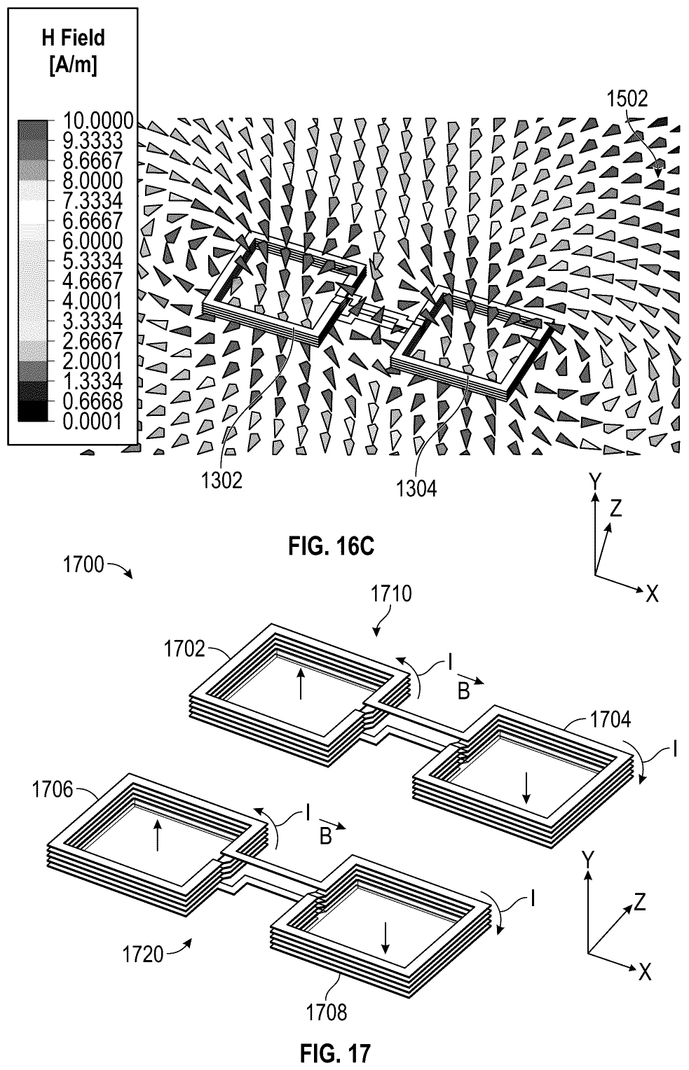

With reference to FIGS. 15A-15C and 16A-16C a coil array 1300 and the magnetic field 1502, 1602 associated with the coil array under various excitations are shown. Based on the principle of superposition, the magnetic field direction at a given position in space above a coil array 1300 will be the vector sum of the contributions from each of the current elements in the array. By controlling the direction of the current “I” (generated by the signal generator 200), the magnetic flux “B” magnitude and direction can be manipulated. The coils of the coil array 1300 may be driven independently, in series, or in parallel in order to achieve the desired magnetic field characteristics. The coil array 1300 may be constructed with as few as two coils or as many as are required to provide the needed magnetic flux “B” direction and magnitude resolution required for the application.

FIG. 15A depicts coil array 1300, where the current flow “I” of the first coil 1302 is in a counter-clockwise direction, and a current flow “I” of the second coil 1304 is also in a clockwise direction. FIG. 15B depicts a side view of a magnetic flux 1502 intensity for the coil array 1300. FIG. 15C is a perspective view of the magnetic flux 1502 intensity for the coil array 1300. Here the magnetic flux 1502 emanates from the first coil 1302 in a negative direction relative to the Y-axis, and the magnetic flux 1502 emanates from the second coil 1304 in a positive direction relative to the Y-axis.

FIG. 16A depicts coil array 1300, where the current flow “I” of the first coil 1302 is in a counter-clockwise direction and a current flow “I” of the second coil 1304 is in a counter-clockwise direction. FIG. 16B depicts a magnetic flux 1602 intensity for the coil array 1300. FIG. 16C is a perspective view of the magnetic flux 1602 intensity for the coil array 1300. Here the magnetic flux 1602 emanates from the first coil 1302 in a negative direction relative to the “Y” axis, and the magnetic flux 1602 also emanates from the second coil 1304 in a negative direction relative to the Y-axis.

Referring to FIGS. 17-19, perspective views of a four-element coil array 1700 for use with the system of FIG. 1, where each of the coils is excited in various directions, is shown. The four-element coil array 1700 includes a first pair of coils 1710 and a second pair of coils 1720. The first pair of coils includes a first coil 1702 and a second coil 1704. The second pair of coils includes a third coil 1706 and a fourth coil 1708. Each of the coils 1702-1708 are configured to be independently steerable, such that the current flow for any of the individual coils may be in a clockwise or a counterclockwise direction, thereby enabling the manipulation of the magnetic flux “B” magnitude and direction.

For example, in FIG. 17 the current flow “I” in the first coil 1702 and the third coil 1706 is in a counter-clockwise direction. The current flow “I” in the second coil 1704 and the fourth coil 1708 is in a clockwise direction. Using superposition, the direction of the magnetic flux “B” for the first pair of coils 1710 and the second pair of coils 1720 is in a positive direction relative to the “X” axis.

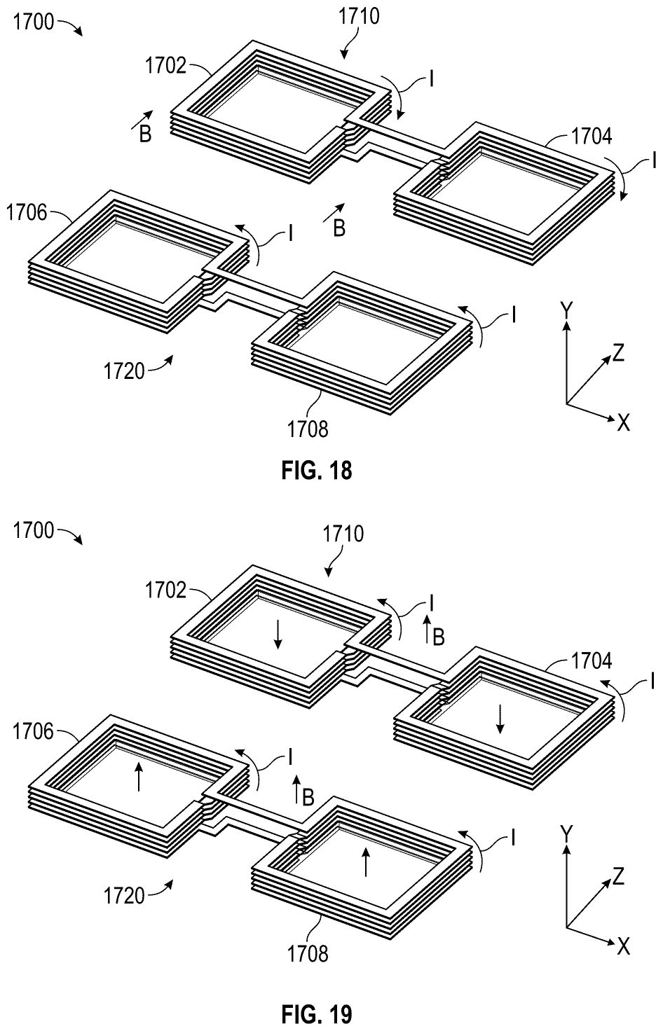

For example, in FIG. 18, the current flow “I” in the first coil 1702 and the second coil 1704 is in a clockwise direction. The current flow “I” in the third coil 1706 and the fourth coil 1708 is in a counter-clockwise direction. Using superposition, the direction of the magnetic flux “B” for the first pair of coils 1710 and the second pair of coils 1720 is in a positive direction relative to the “Z” axis.

For example, in FIG. 19 the current flow “I” in all of the coils 1702-1708 is in a counter-clockwise direction. The resulting direction of the magnetic flux “B” for the first pair of coils 1710 and the second pair of coils 1720 is in a positive direction relative to the “Y” axis.

It is contemplated that the current “I” magnitude for each of the individual coils 1702-1708 may include a positive or negative value, and the current “I” direction for each of the individual coils may vary in a clockwise or counterclockwise manner, in order to enable the steering of the magnetic flux “B” direction and/or magnitude to suit the application.

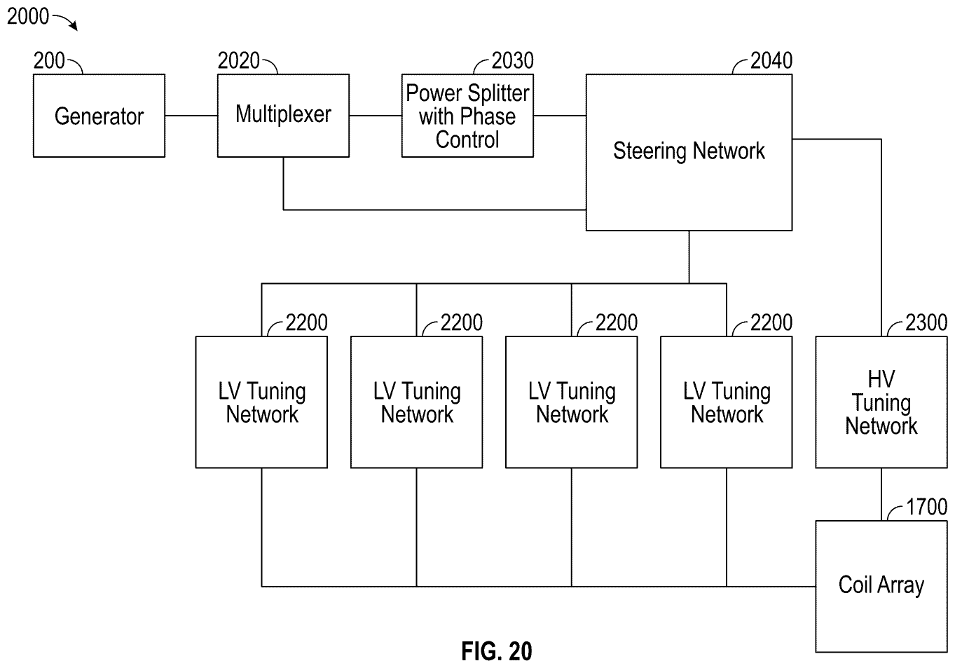

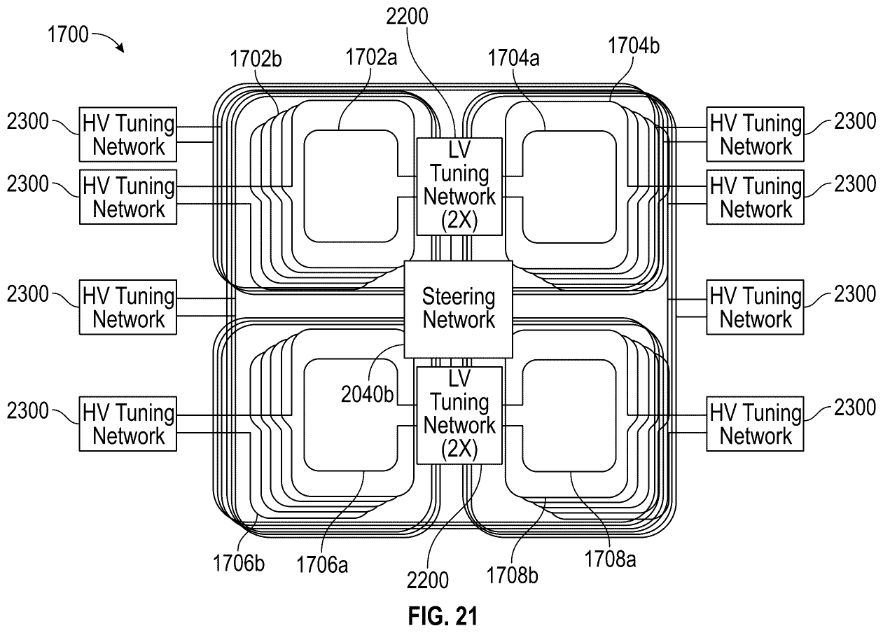

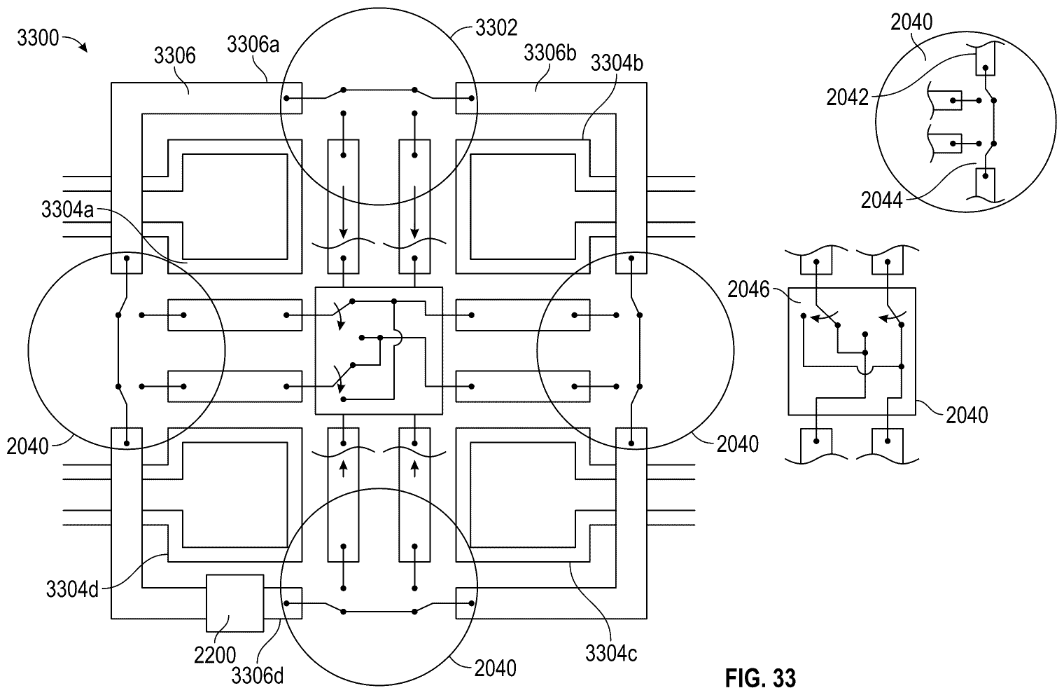

Referring to FIG. 20, a block diagram for a system 2000 for dynamically configuring a secondary air-core coupled coil and exciting magnetic fields in multiple directions, which may be used as the antenna 300 (FIG. 3) of the detection system 10 (FIG. 1), is shown. The disclosed system both provides adequate magnetic field strength to communicate with an RFID tag at an acceptable read range as well as enabling communication with the RFID tag that has an arbitrary, non-optimal orientation with respect to the antenna 300 (FIG. 3). The system 2000 generally includes a multiplexer 2020, a phase splitter 2030 with phase control, a low voltage (LV) steering network 2040 (FIG. 33) which enables current steering of a secondary coil through a primary coil for a secondary coil configuration best suited to excite an RFID tag 100 (FIG. 1) in a given orientation, a coil tuning network 2200 configured to tune a quality factor “Q” and/or an operating frequency of the primary coils 1702a, 1704a, 1706a, 1708a of the coil array 1700 (FIG. 21), and a coil termination network 2300 configured to enable and/or disable one or more secondary coils 1702b, 1704b, 1706b, 1708b of coil array 1700. The term operating frequency may include a band of frequencies that a component (e.g., the antenna 300) is designed to operate within.

For applications that provide both detection and identification of tagged surgical items, it may be desirable to implement a larger static antenna array 1300 (FIG. 1) either embedded into a surgical table or integrated with a surgical table mattress. The static antenna array 1300 (FIG. 1) has the distinct advantage of reducing the variability of a scan cycle frequently associated with a handheld antenna. This variability is a result of the differences in operator scanning techniques. A static antenna array 1300 is also appealing as it offers the option of increasing the size of the scanning accessory when compared to a handheld antenna. The increased size has the benefits of reducing the amount of time required to scan, as well as taking advantage of the inherent performance advantages from a read range perspective relating to the increased coil area.

For typical RFID applications, where communication between an antenna 300 (FIG. 3) and an RFID tag 100 (FIG. 1) is accomplished via coupled magnetic fields, the read range and sensitivity to antenna 300 orientation is a direct function of the coil areas and orientation. Accordingly, a larger coil will generally perform better than a smaller coil in terms of the range where communication is possible, as well as the relative orientation between the antenna 300 (FIG. 3) and the RFID tag coil (not shown). For example, when performing detection and identification of tagged surgical items, the acceptable physical sizes of both the antenna 300 (FIG. 3) and the RFID tag coil are smaller than those that would provide the optimal read range performance and reduced sensitivity to orientation. In the case of the RFID tag 100 (FIG. 1), the acceptable physical size of the tracked tag component is much smaller than ideal.

As mentioned previously, this disclosure combines the advantages of the air-core coupled secondary construction (FIG. 3) with the benefits of field direction control via current steering and control in a multi-coil element array (FIG. 18). While the following describes an array of four elements for simplicity, the array dimensions (N×M) are not limited and may be extended to provide the coverage needed in the target form-factor. Additionally, the array elements may take on a fractal structure, where arrays of smaller coil elements and air-core coupled secondaries may act as a single primary coil in a higher-level array.

Referring to FIG. 21, a block diagram of a four-element coil array 1700, for use with the detection system 10 of FIG. 1, is shown. For this example, the coil array 1700 includes four (2×2) primary coils 1702a, 1704a, 1706a, 1708a and five discrete air-core coupled secondary coils 1702b, 1704b, 1706b, 1708b. Each primary coil 1702a, 1704a, 1706a, 1708a may be operably connected to an associated coil tuning network 2200. Each secondary coil 1702b, 1704b, 1706b, 1708b may be operably connected to an associated configurable coil termination network 2300 and a high voltage (HV) steering network 2040b.

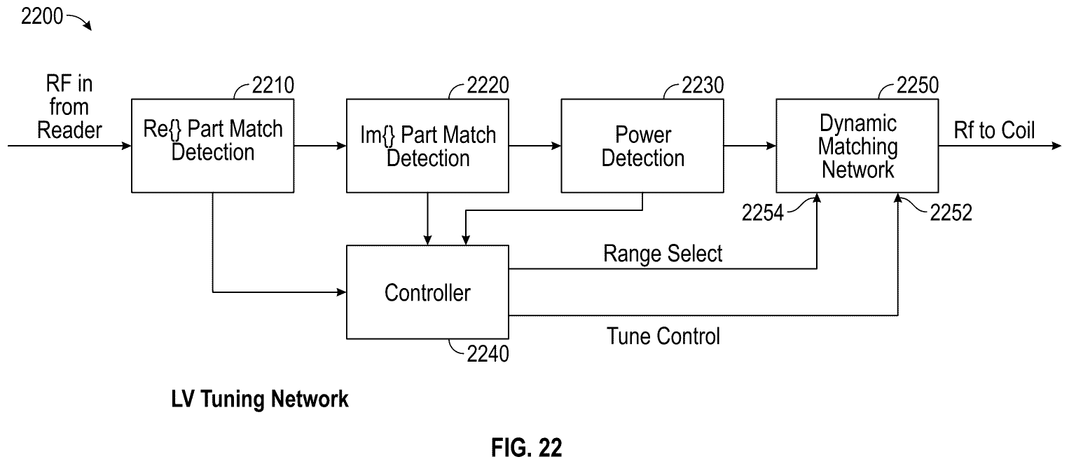

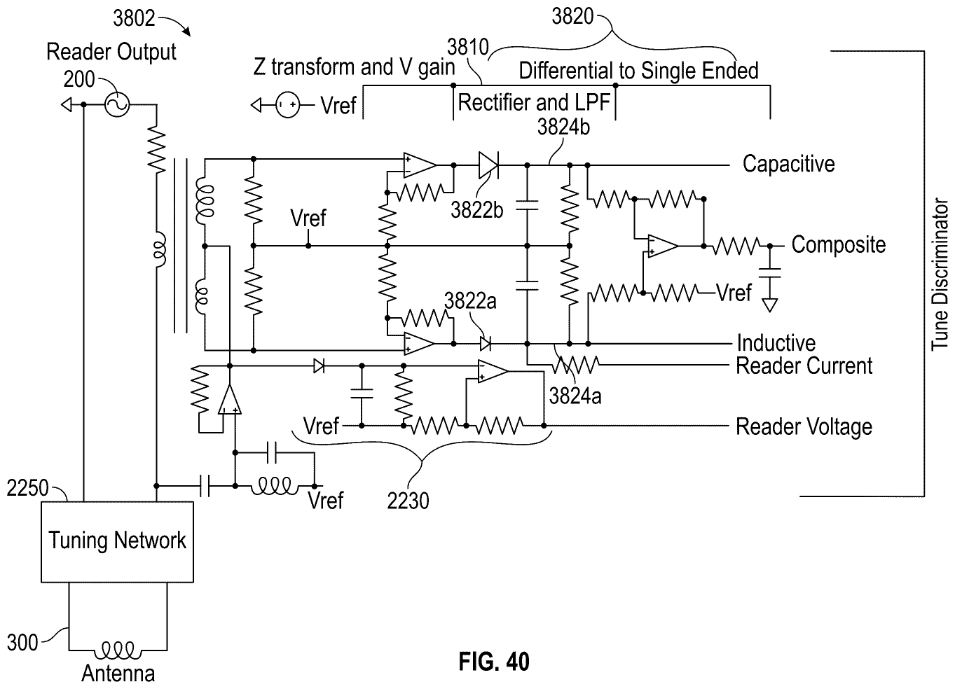

FIG. 22 is a block diagram of a coil tuning network 2200 for use with the coil array 1700 of FIG. 20. The coil tuning network 2200 generally includes a real part match detection network 2210, an imaginary part match detection network 2220, a power detection network 2230, a dynamic matching network 2250, and a controller 2240. RF signals may be represented by complex numbers and typically include a real part (e.g., in-phase) and an imaginary part (e.g., quadrature). The configurable termination networks 2200, 2300 may be controlled by the controller 2240. The real part match detection network 2210 receives an input RF signal which was generated by the generator 200 (FIG. 1) and is configured to detect the real part of the RF input signal (e.g., a signal indicating a real part of the RF signal). The imaginary part match detection network 2220 is configured to detect the imaginary part of the RF input signal (e.g., a signal indicating an imaginary part of the RF signal). In aspects, the real part match detection network 2210 and/or the imaginary part match detection network 2220 may be implemented using an I/Q modulator. The power detection network 2230 is configured to detect the RF power level from the RF signal and provide that power level to the controller 2240 for further processing. The power detection network 2230 may include but is not limited to, for example, an RF detector diode. In aspects, instead of, or in addition to detecting the RF power level, the power detection network 2230 may be configured to detect voltage and/or current using a voltage and/or current sensor (e.g., a coil current detector). In aspects, the power detection network 2230 may be located at the output of the dynamic matching network 2250 where it may be configured to determine a field strength generated by the coil. For example, the power detection network 2230 may be a coil current detector located at the output of the dynamic matching network 2250. In aspects, power detection network 2230 may determine the RF power by calculating a point by point product based on the acquired voltage and/or current. In aspects, power detection network 2230 may determine RF power by post processing the reader current and reader voltage signals from FIG. 40 in conjunction with power factor correction using the composite capacitive and inductive signals.

The controller 2240 is configured to receive signals from the real part match detection network 2210, the imaginary part match detection network 2220, the power detection network 2230, and control the dynamic matching network 2250 based on the detected signals. The controller 2240 includes a processor and memory, and is configured to execute instructions stored on the memory.

The dynamic matching network 2250 is configured to tune the quality factor “Q” and/or resonant frequency of the primary coil 1702a, 1704a, 1706a, 1708a of the coil array 1700 (FIG. 21). The dynamic matching network 2250 may include but is not limited to, for example, capacitors, inductors, varactors, PIN diodes, and/or resistors. The dynamic matching network 2250 may include a range select input 2254 configured to receive a range select signal from the controller 2240 and, based on the received range select signal, output an RF control signal that changes the resonant frequency range of the primary coil(s) 1702a, 1704a, 1706a, 1708a by presenting a dynamic load to the primary coil(s) 1702a, 1704a, 1706a, 1708a. The dynamic matching network 2250 may include a tune control select input 2252 configured to receive a tune select signal from the controller 2240 and, based on the received tune select signal, output an RF control signal that changes the resonant frequency of the primary coil(s) 1702a, 1704a, 1706a, 1708a by presenting a dynamic load to the primary coil(s) 1702a, 1704a, 1706a, 1708a. In aspects, the primary coils 1702a, 1704a, 1706a, 1708a. may be driven independently or in combinations of series and/or parallel elements as needed to present the detection system 10 and coil tuning network 2200 with an optimized load for a given configuration.

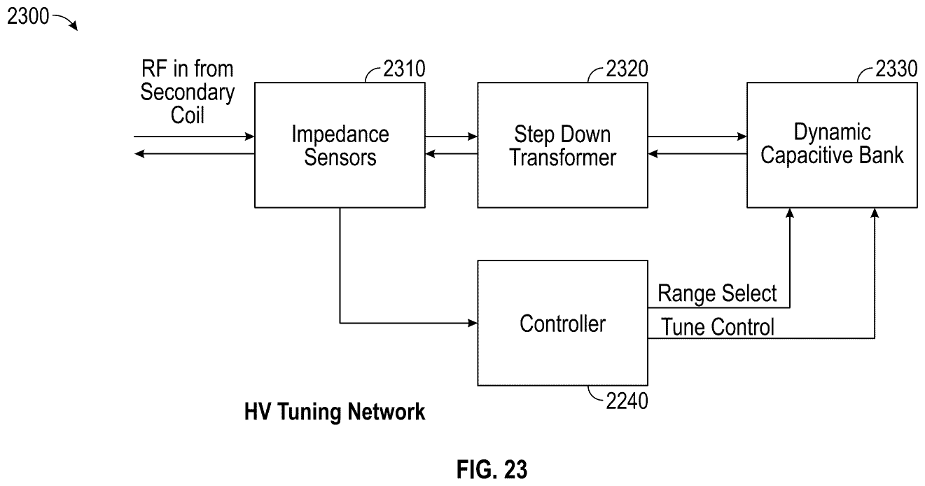

FIG. 23 is a block diagram of a coil termination network 2300 for use with the coil array 1700 of FIG. 20. The coil termination network 2300 generally includes an impedance sensor 2310 configured to sense the impedance of the secondary coil(s) at the target frequency of operation, 1702b, 1704b, 1706b, 1708b, a step-down transformer 2310 configured to step down the voltage of the secondary coil(s), 1702b, 1704b, 1706b, 1708b, a dynamic capacitive bank 2330, configured to provide a switchable match to the secondary coil(s), 1702b, 1704b, 1706b, 1708b via the step-down transformer 2320, and a controller 2240. It is contemplated that the same and/or a different controller may be used than is used for the coil tuning network 2200.

The coil termination network 2300 is configured to enable or disable discrete secondary coils 1702b, 1704b, 1706b, 1708b of the coil array 1700. The coil termination network 2300 may eliminate a resonant network (open circuit), it may short a coil element, or it may terminate the coil based on a loading condition (by switching in different value capacitors) that shifts the resonant characteristics to a region in the frequency domain that makes it appear to be out-of-circuit from a magnetic field perspective and/or from the loading it presents to the detection system 10 (FIG. 1) and coil tuning network 2200.

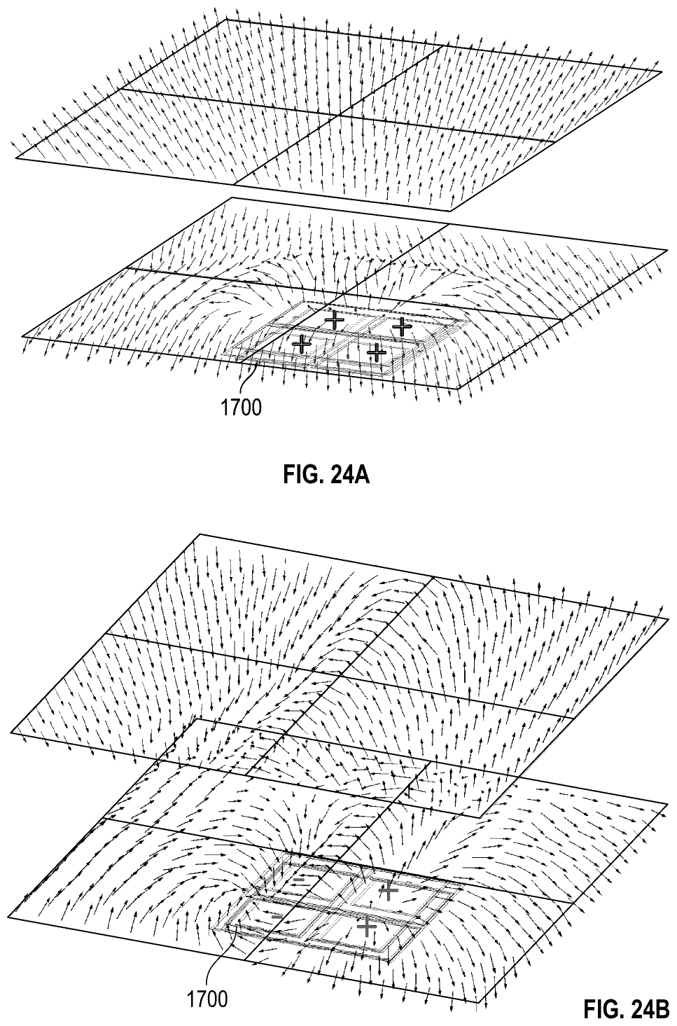

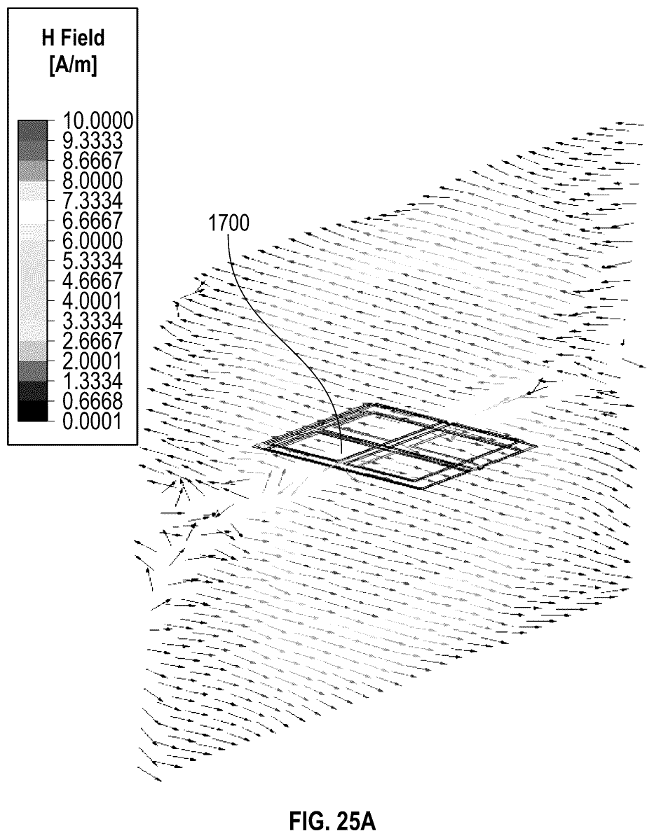

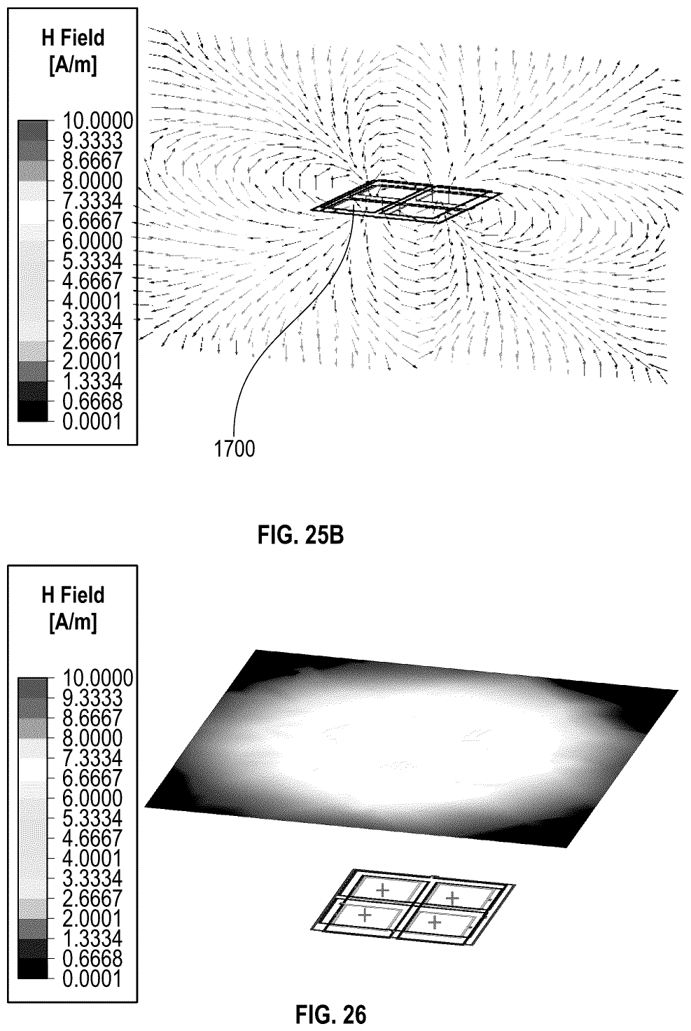

FIGS. 24A and 24B depict the relative magnetic field “H” intensity and vector directions for a four-element array 1700. FIG. 24A depicts the magnetic field “H” vector plot when two pairs of coils are driven in phase. FIG. 24B depicts the magnetic field “H” vector plot when two pairs of coils are driven in opposite phase. FIG. 25A depicts a vector field intensity and direction for a “horizontal” current steering configuration in the X-Z plane. FIG. 25B depicts a vector field intensity and direction for a “horizontal” current steering configuration in the X-Z plane. FIG. 26 depicts an isometric view of the magnetic field “H” strength in a configuration where the secondary coil is terminated to eliminate a contribution in a vertical orientation configuration.

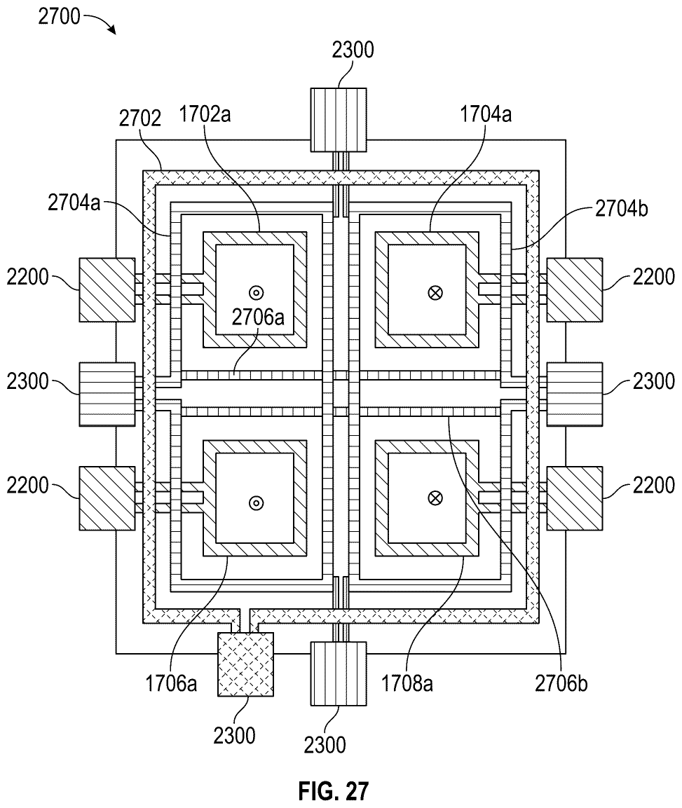

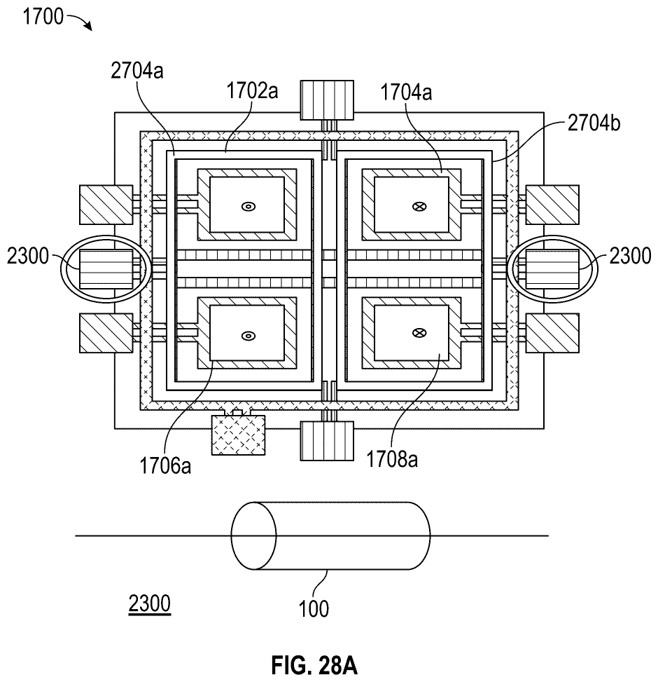

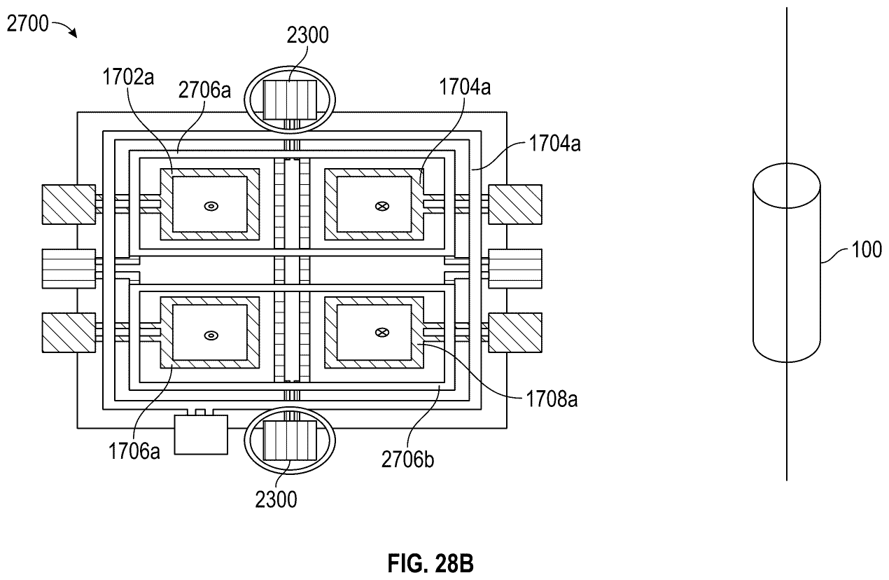

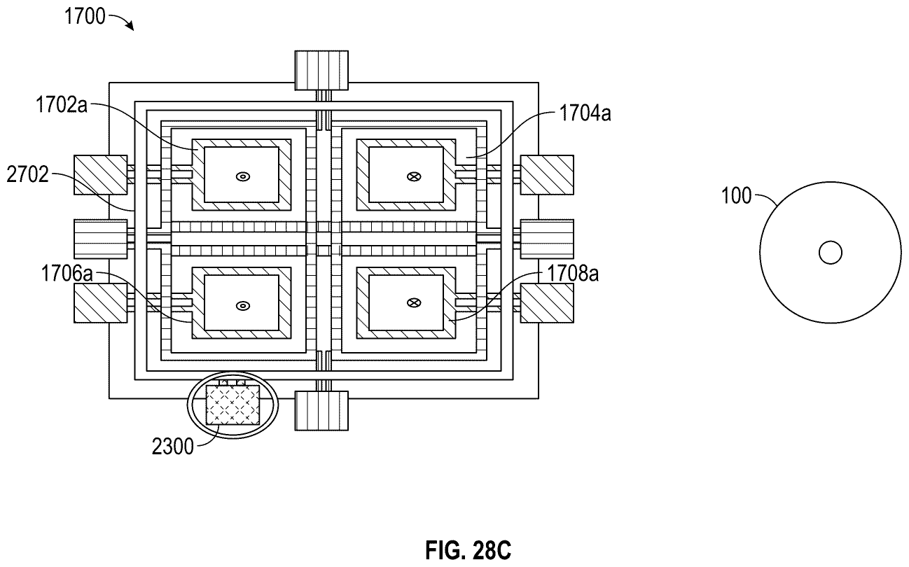

Referring to FIG. 27, an illustration of a four-element coil array 2700 for use with the detection system 10 of FIG. 1 is shown. For this example, the coil array includes four (2×2) primary coils 1702a, 1704a, 1706a, 1708a and five discrete air-core coupled secondary coils 2702, 2704a, 2704b, 2706a, 2706b. Each primary coil 1702a, 1704a, 1706a, 1708a may be operably connected to an associated coil tuning network 2200. Each secondary coil 2702, 2704a, 2704b, 2706a, 2706b may be operably connected to an associated configurable coil termination network 2300. The configurable coil termination networks 2200, 2300 may be controlled by the controller 2030. The controller 2030 may perform a scan cycle which may include a series of different configurations intended to excite and communicate with RFID tags 100 in arbitrary orientations. The optimal Cartesian orientations in two dimensions are shown for each discrete secondary coil configuration in FIG. 28A-28C.

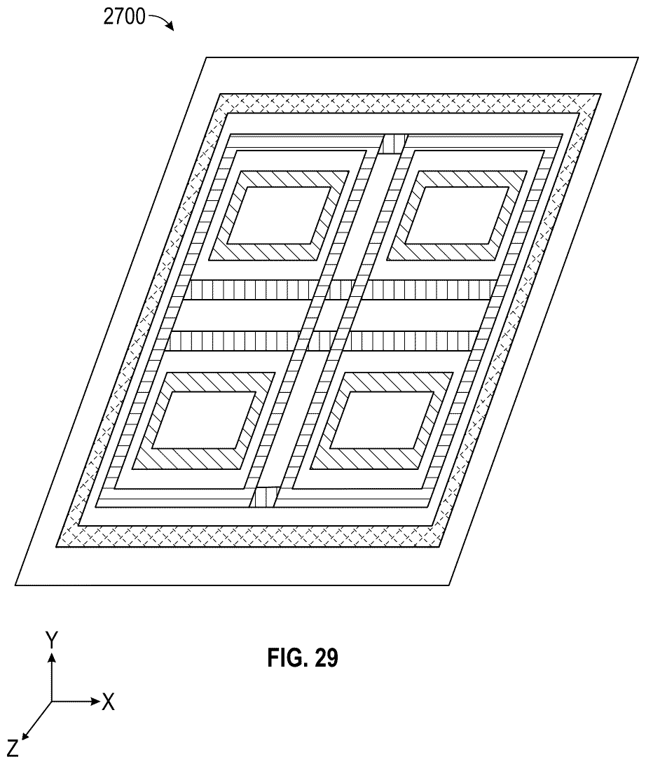

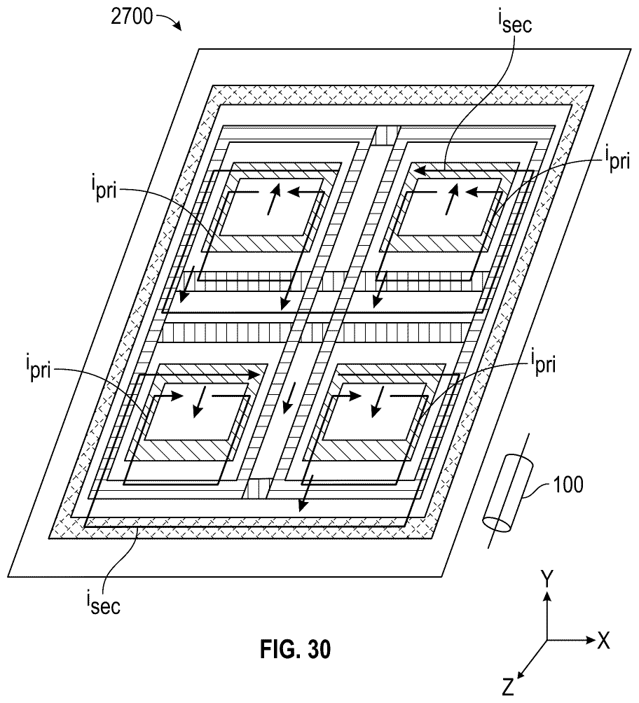

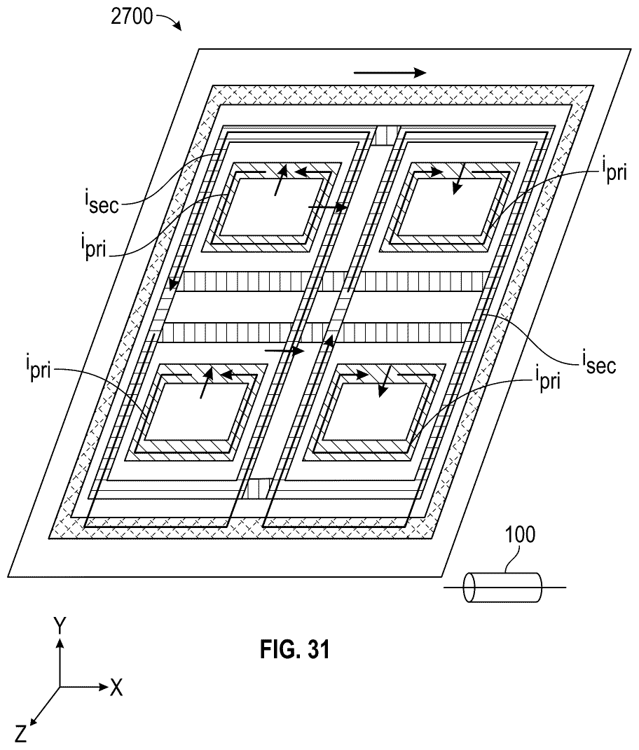

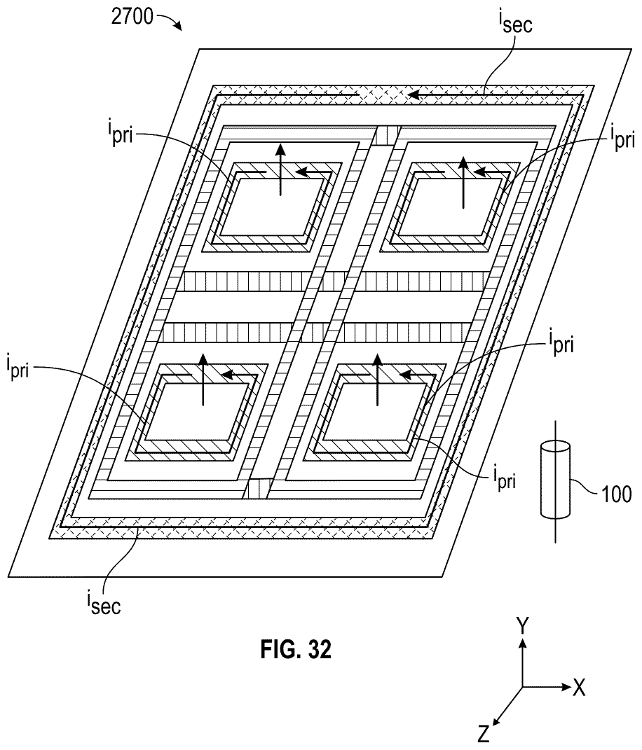

FIG. 29 depicts an isometric view of the coil array 2700 of FIG. 27. Referring to FIGS. 30-32, the primary coil current iₚᵣᵢ directions, configurable secondary coil current i_{sec} directions, steering network 2040 configuration, and optimal RFID tag 100 orientation directions for three possible configurations of the coil array 2700 of FIG. 27 are shown. For example, in FIG. 30, the primary coil current iₚᵣᵢ of primary coils 1702a and 1704a (FIG. 28B), and the secondary coil current i_{sec}, of secondary coil 2706a flow in a counter-clockwise direction, while the primary coil current iₚᵣᵢ of primary coils 1706a and 1708a and secondary coil current i_{sec} of secondary coil 2706b flow in a counter clockwise direction, thus steering the magnetic flux “B.”

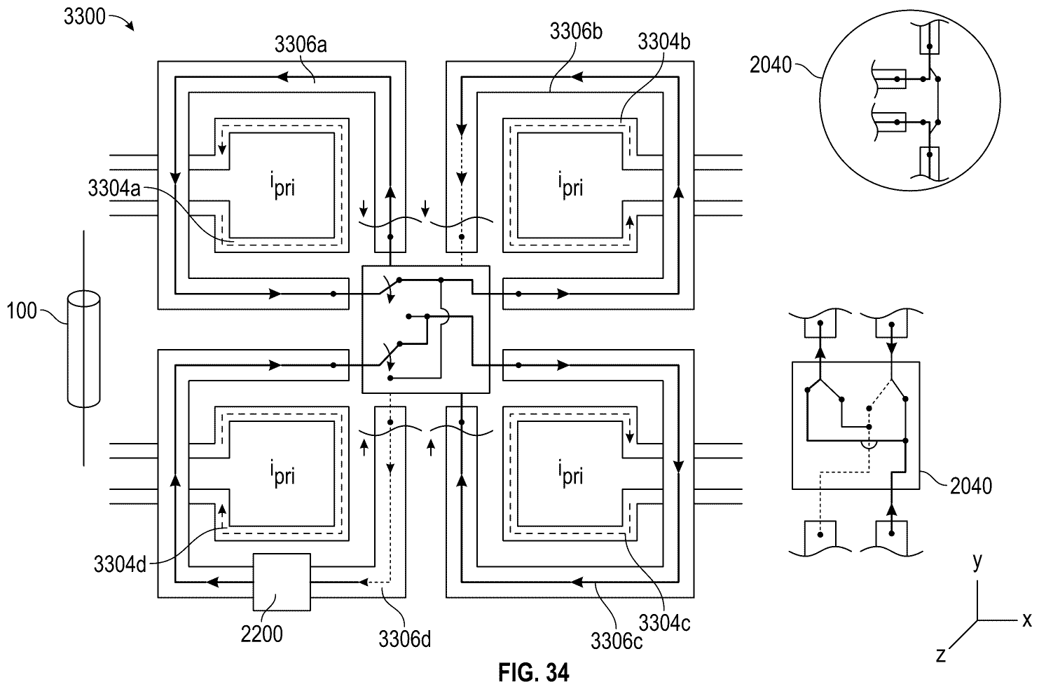

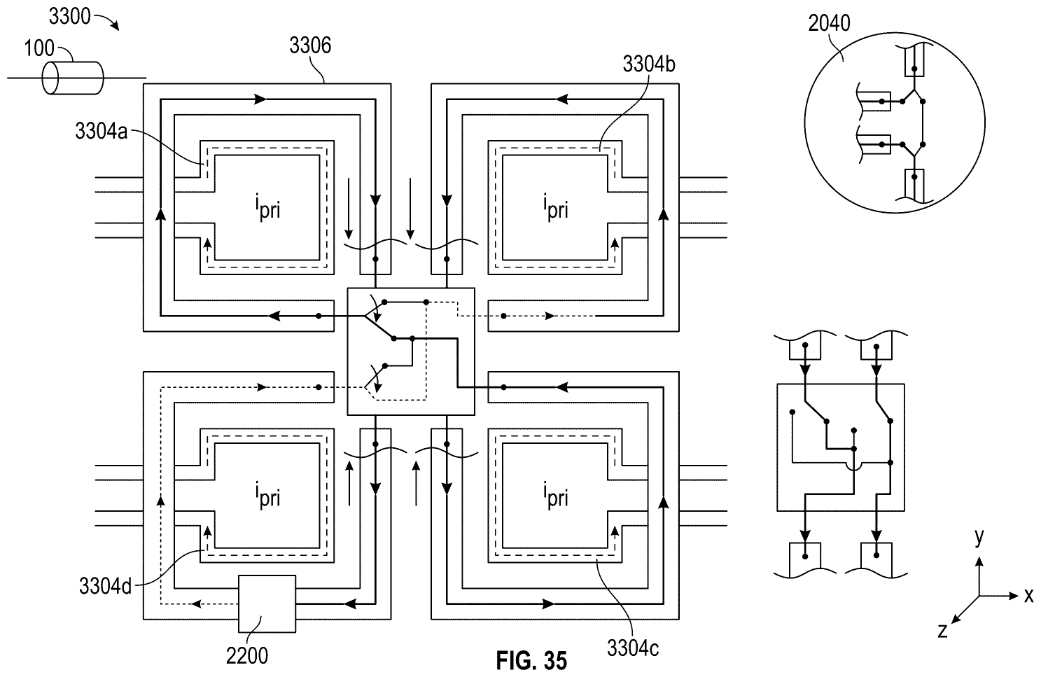

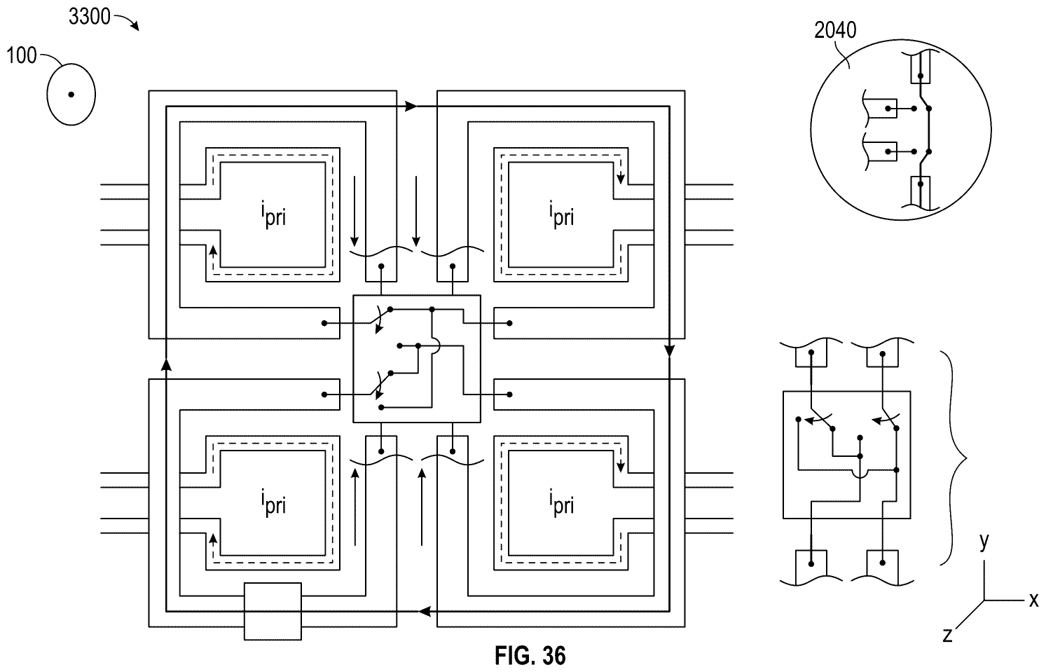

With reference to FIGS. 33-36, the system 3300 may include a configurable air-core coupled secondary coil 3306. In aspects, a single or array of larger, more complex secondary coupled coils may be used. The coil configurations may be determined via specialized steering networks 2040, 2040b that direct the coupled currents through the coil array elements (e.g., secondary coil sections 3306a, 3306b, 3306c, 3306d) that result in field vector directions capable of exciting RFID tags 100 at arbitrary orientations. In aspects, the steering network 2040 may include a switching network 2042, 2044. The switching network 2042 may include, for example, PIN switching diodes or other electronically actuatable switches. In aspects, the steering networks 2040, 2040b may include a switching network 2044 on a top surface and on a bottom surface of a printed circuit board 2046.

For example, the system 3300 may include four (2×2) primary coils 3304a, 3304b, 3304c, 3304d, and a configurable air-core coupled secondary coil 3306. Each primary coil 3304a, 3304b, 3304c, 3304d has an associated coil tuning network 2200. The configurable air-core coupled secondary coil 3306 generally includes a first section 3306a, a second section 3306b, a third section 3306c, and a fourth section 3306d. It is contemplated that any suitable number of sections 3306a, 3306b, 3306c, 3306d may be used. The configurable air-core coupled secondary coil 3306 has a configurable coil termination network 2300.

The steering network 2040 is used to configure the configurable air-core coupled secondary coil 3306 configuration best suited to excite an RFID tag 100 in a given orientation. The configurable termination 2200 and steering networks 2040 are controlled by the controller 2240. The steering networks 2040 may be located in the middle of and/or between the various sections of the configurable air-core coupled secondary coil 3306 to enable the switching in and out of various sections 3306a, 3306b, 3306c, 3306d of the configurable air-core coupled secondary coil 3306.

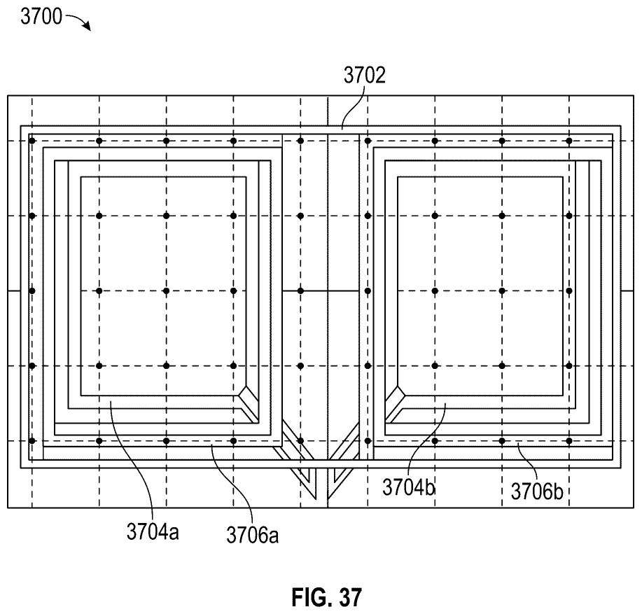

FIG. 37 depicts an example of a two-element coil array 3700. The two-element coil array 3700 includes two primary coils 3706a and 3706b, and three configurable secondary coils 3702, 3704a, 3704b. In aspects, the system 2000 may include a magnetic shield (not shown) positioned either on the opposite side of the coil array 3700 from the intended read direction or coincident with primary and/or secondary coil elements. The shield (not shown) may be made using ferrite sheet and/or similar high permeability materials.

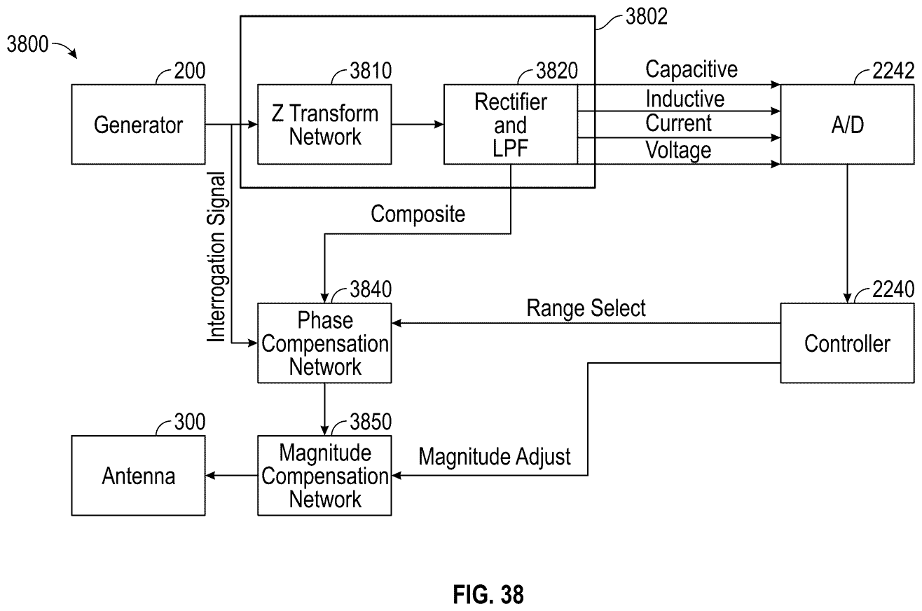

Referring to FIG. 38 a high-level block diagram for a system 3800 for real-time dynamic tuning of an antenna coil (e.g., primary coil 302 of FIG. 3) to a generator 200 of the interrogation and detection system 10 of FIG. 1, is shown. The system 3800 for real-time dynamic tuning generally includes a tuning discriminator 3802 configured to generate signals used for determining the real and imaginary parts of the energizing signal, an analog to digital converter (A/D) 2242 configured to digitize signals from the discrimination network 3820, a phase compensation network 3840 configured to dynamically tune the matching impedance phase, a magnitude compensation network 3850 configured to dynamically tune the matching impedance, and a controller 2240. The system 3800 may be implemented as a primary side tuning network 410 (FIG. 4), e.g., a tuning network 410 disposed between the generator 200 and the antenna 300 (FIG. 3). The tuning network 2250 (FIG. 40) may be used in single coil antennas or in multiple coil antennas.

Typically, autotuning is conducted as a discrete step or state in the system operation. Autotuning is done relatively infrequently and generally requires disabling the RFID communications channel. In surgical applications, that is an unacceptable scenario, as the antenna loading is potentially changing constantly based on tissue properties, and it would be impractical to require a re-tuning step multiple times during a scanning sequence, especially for hand-held scanning accessories.

For applications that provide both detection and identification of tagged surgical items, unique technology implementations, and tuning methods are required to adapt the dynamic loading conditions presented in the operating room surgical setting. Specifically, for RFID applications, a load presented to an antenna 300 or an antenna array 1700 (FIG. 17) is a function of the material properties of the various structures in close proximity to the antenna structures. These structures and their material properties are constantly changing due to multiple factors, including physical size, physical shape, physical orientation, distance from the antenna 300 or antenna array 1700 (FIG. 17), homogeneity, or lack there-of, and/or boundary discontinuities.

For optimal power transfer into the field, the interface between the generator 200 and the antenna 300 needs to be tuned in real-time or near real-time in order to compensate for a continuously variable loading state.

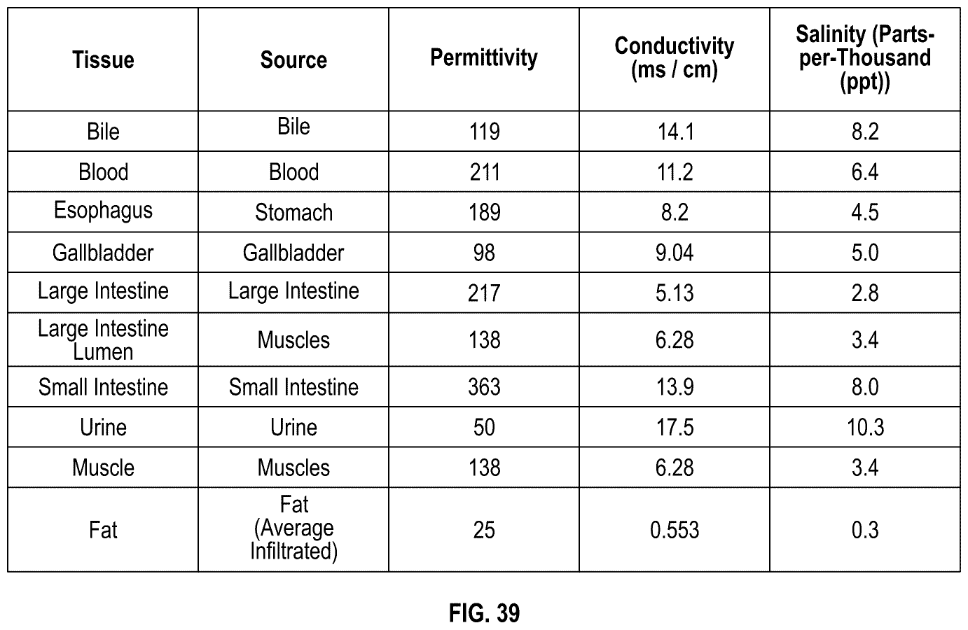

Referring to FIG. 39, a table depicting the permittivity and conductivity of various tissue types is shown. Example permittivity and conductivity of various tissue types may be found at https://itis.swiss/virtual-population/tissue-properties/database/dielectric-properties/, which is incorporated by reference. The propagation and attenuation of the RF energy that is generated by the generator 200 and antenna 300 are dependent on the material properties of the structures present in the field, so adaptation to this variable is required. The disclosed technology can compensate for variation in the permittivity and shift in resonance typically found with organic tissue loading conditions. For example, if the tissue type is a muscle, then the permittivity is approximately 138 F/m, and the conductivity is 6.28 mS/cm. However, if the tissue type is small intestinal tissue, then the permittivity is approximately 363 F/m, and the conductivity is 13.9 mS/cm, which can present a very different condition for the antenna 300.

Referring to FIG. 40, a diagram for the tuning discriminator 3802 of the system 3800, which is configured to generate signals used for determining the real and imaginary parts of the energizing signal, is shown. The tuning discriminator 3802 generally includes an impedance (Z) transform network 3810, a rectifier 3822a, 3822b, a low pass filter (LPF) 3824a, 3824b, and a power detector 2230. The tuning discriminator 3802 analyzes the energizing signal generated by the generator 200 and generates the signals used to calculate the impedance magnitude, such as an energizing signal current and an energizing signal voltage (e.g., a real part). The tuning discriminator 3802 analyzes the difference in phase between the voltage and the current of the energizing signal. For example, a positive phase relative to voltage (inductive) forward biases the diode 3822a in the inductive leg and reverse biases the diode 3822b in the capacitive leg. In another example, a negative relative phase (capacitive) forward biases the diode 3822b in the capacitive leg and reverse biases the diode 3822a in the inductive leg. A difference between the two biased voltages is determined and the result is positive relative to Vref for inductive signals and negative relative to Vref for capacitive signals. Either leg of this network (capacitive of inductive) may be used as a representation of the actual current flowing to the load, which may get additional post processing. For example, an RMS version of the current, an RMS version of the voltage, and power factor correction may be used in post processing for power measurement. The energizing signal current and the energizing signal voltage signals are communicated to the controller 2240 via the A/D converter 2242 where the load impedance magnitude is calculated. The tuning discriminator 3802 further analyzes the energizing signal generated by the generator 200 and generates the composite, capacitive, and inductive signals (e.g., an imaginary part). Which are all then communicated to the controller 2240 via the A/D converter 2242. Additionally, the composite signal is passed to the phase compensation network 3840 (FIG. 38). In aspects, the composite reference signal may be pre-processed by the controller 2240 to compensate for non-linear effects. The tuning discriminator 3802 provides the advantage of significantly improving analog to digital converter performance.

The Z-transform network 3810 is configured to analyze the energizing signal from the signal generator 200 for identification of the imaginary part of the energizing signal. This frequency domain representation of the energizing signal is then communicated to the rectifier 3822a, 3822b for rectification. The rectifier 3822a, 3822b may include one or more diodes which convert the alternating current signal (AC) of the energizing signal to a direct current (DC) signal (with some AC artifacts). This DC signal is then filtered by the LPF 3824a, 3824b to remove any residual AC artifacts (e.g., ripple and/or the fundamental frequency of the energizing signal). The LPF 3824a, 3824b, may include a passive (e.g., an R/C network) or an active configuration (e.g., an integrator). The output of the LPF 3824a, 3824b, includes the capacitive signal, the inductive signal, and/or the energizing signal current. The capacitive signal and the inductive signal may be combined into the composite signal by a differential to single-ended converter (e.g., a difference amplifier). The composite signal may be used by the controller 2240 for tuning the impedance magnitude provided to the antenna 300.

The power detector 2230 detects the power level of the energizing signal (e.g., about 1 W to about 10 W typically) by sampling the energizing signal (e.g., by a coupler), rectifying the sampled signal (e.g., by a diode), and processing the signal by adding gain before communicating it to the controller 2240 as the energizing signal voltage.

The controller 2240 is configured to determine the optimal impedance match for the antenna 300 based on the capacitive signal, the inductive signal, the energizing signal current, the energizing signal voltage, and/or the composite signal. The controller 2240 automatically tunes the impedance phase and impedance magnitude of the antenna 300 in real-time based on the determined optimal impedance match. In aspects, the controller 2240 may include multiple independent control loops or may be comprised of several nested control loops for improved stability. The nesting order may be determined by the loop time-constant and may be arranged in multiple ways that optimize the total system response.

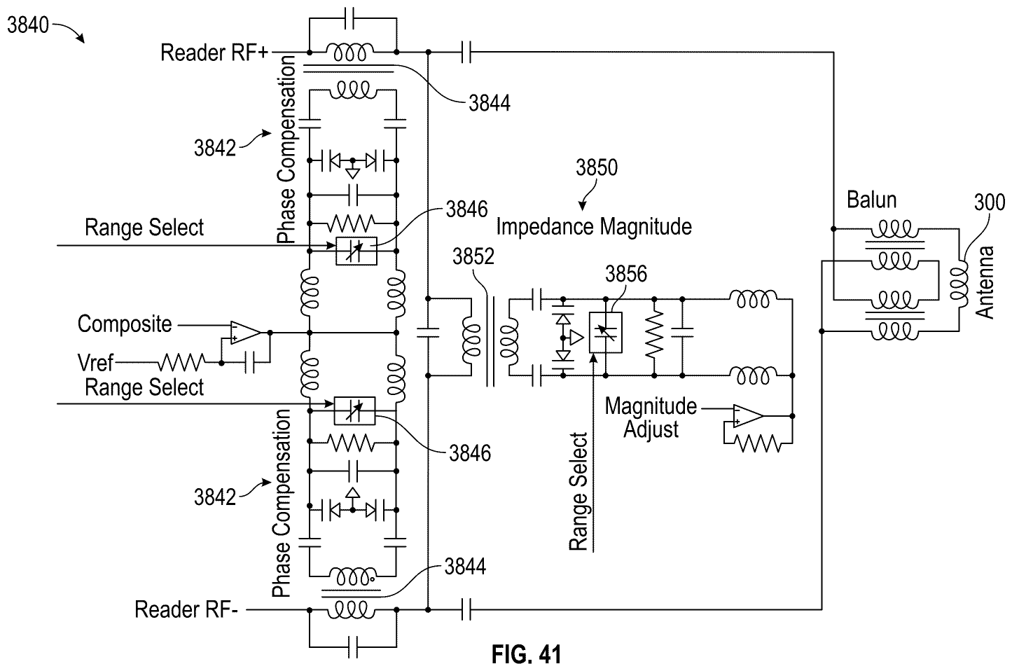

With reference to FIG. 41, the phase compensation network 3840 and magnitude compensation network 3850 are shown. The phase compensation network 3840 is configured to tune the phase of impedance match in real-time. The phase compensation network 3840 generally includes one or more resistors, one or more diodes, and a voltage-controlled dynamic capacitance element 3846 configured to be controlled by the range select signal from the controller 2240. In aspects, the phase compensation network 3840 may include transformers 3844 (e.g., a magnetic-based transformer, and/or a directional coupler) in order to reduce the voltage across the dynamic capacitance elements 3846. For example, the voltage may be reduced to levels typically found in commercial off-the-shelf options, as well as providing isolation from the signal generator 200, the antenna 300, and/or the compensation networks 3840, 3850. The use of the transformers 3844 enables ground plane referenced control schemes. Only a single feedback channel is illustrated, but this concept extends to implementations that use individual channels for each voltage-controlled capacitance element. Furthermore, even though the voltage-controlled dynamic capacitance element 3846 may include a varactor diode and/or a voltage-controlled capacitor.

The magnitude compensation network 3850 is configured to tune the magnitude of impedance match provided to the antenna 300, in real-time. The magnitude compensation network 3850 generally includes one or more resistors, one or more diodes, and a voltage-controlled dynamic capacitance element 3856 configured to be controlled by the range select signal from the controller 2240. The magnitude adjust signal is communicated from the controller 2240 to the magnitude compensation network 3850 and causes the magnitude compensation network 3850 to increase or decrease the magnitude of the impedance match. In aspects, the magnitude compensation network 3850 may include one or more transformers 3852 (e.g., a magnetic-based transformer) in order to reduce the voltage across the dynamic capacitance element 3856. In aspects, the magnitude compensation network 3850 may be used for the tuning networks 410 and 420 of FIG. 4.

For the case of an air-core coupled secondary coil 420 (e.g., FIG. 4), the secondary coil 304 benefits from real-time tuning as well. This has the effect of maintaining the bulk of the primary referenced reactive impedance and reduces the burden imposed on the primary side tuning network 410 (FIG. 4). Additionally, secondary coil automatic tuning retains the optimal secondary coil 304 (FIG. 4) resonant frequency, which improves the secondary coil 304 quality factor “Q” benefits.

The field strength at a specific distance or “read range” from the antenna 300 is a function of several physical parameters. These parameters may include the air gap that exists between the antenna 300 (FIG. 1) and the surface of the conductive medium (the patient), the thickness of the conductive medium (saline depth, tissue thickness), and the conductivity of the conductive medium (tissue). It can also be shown that the relative quality factor “Q” of the antenna 300 can be used to quantify these parameters such that through the quality factor “Q” alone, we can establish the necessary system operational parameters to achieve a target field strength for a fixed height above the coil surface. The challenge arises from our ability to quantify the Q-factor for an antenna coil in real-time and then correlate that quality factor “Q” with the appropriate reader output to achieve the target field strength. Quality factor “Q” is a function of the air gap, the saline depth, and the salinity.

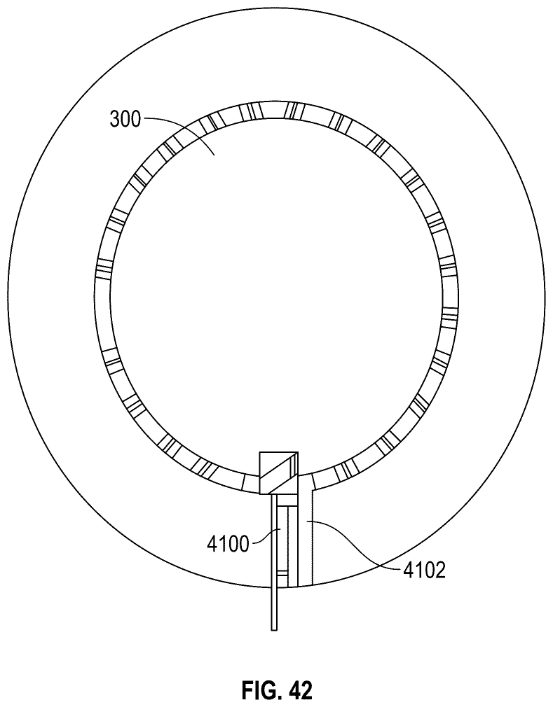

Referring to FIG. 42, an integrated Q-factor sensor for use with the system 3800 for real-time dynamic tuning, is shown. Generally, for conductive mediums with a high saline content, there is a rapid fall-off in the Q-factor as the air-gap is reduced between the RFID tag 100 and the antenna 300. This is because the conductive medium shunts current, as opposed to the current flowing through the intended coil, before the conductive medium has a chance to conduct current through the antenna's current elements (e.g., the antenna coil(s), see FIG. 3). The field strength in space is the integral sum of each discrete current element, and we are, in essence, short-circuiting the coil with the conductive medium. Quality factor “Q” is not easily measured directly. It can be shown that the coil current for a given power setting is a valid proxy for the quality factor “Q.” By embedding an air-core coupled magnetic field pick-up coil 4100 in a coil feed network 4102, it is possible to measure the coil current (and/or voltage) without degrading the coil performance.

An additional advantage to measuring the antenna coil quality factor “Q” for use in the determination of the required output power needed to achieve a target field strength at a target distance from the antenna 300 relates to the information it provides in terms of the scanning technique for hand-held scanning accessories. The controller 2240 can be programmed with target limits for the quality factor “Q,” where the physical proximity characteristics of the scanning accessory with respect to the patient tissue can be established. These limits may be used to provide real-time feedback to the operator in order to improve the scanning efficiency and effectiveness. Specifically, the quality factor “Q” may be interpreted to indicate whether or not the antenna 300 is too close (air gap) to the patient, it can indicate if the antenna 300 is too far away, and the system 3800 can indicate if the tissue load on the accessory is outside of the acceptable range. Furthermore, it is possible that the quality factor “Q” in conjunction with the coil inductance (the phase) and coil magnitude may indicate the presence of interfering objects in the field, like metal instrumentation.

In aspects, the system 3800 may utilize a single voltage or current sensor alone, with the compensation routine adjusting a resonance of a secondary coil of the antenna 300 in order to maximize the sensed parameter. The simplified routine may incorporate a simple set-point dither technique, where the antenna 300 resonant frequency and/or Q-factor is adjusted using binary reduction based on the polarity of the sensed parameter (voltage and/or current) delta.

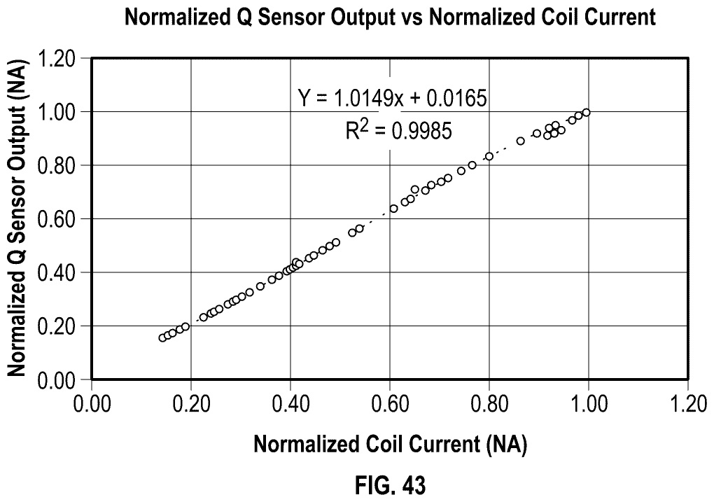

Referring to FIG. 43, a graph illustrating the linear output of the “Q”-factor sensor of FIG. 41 is shown. The graph depicts the normalized “Q” sensor output vs. antenna coil current.

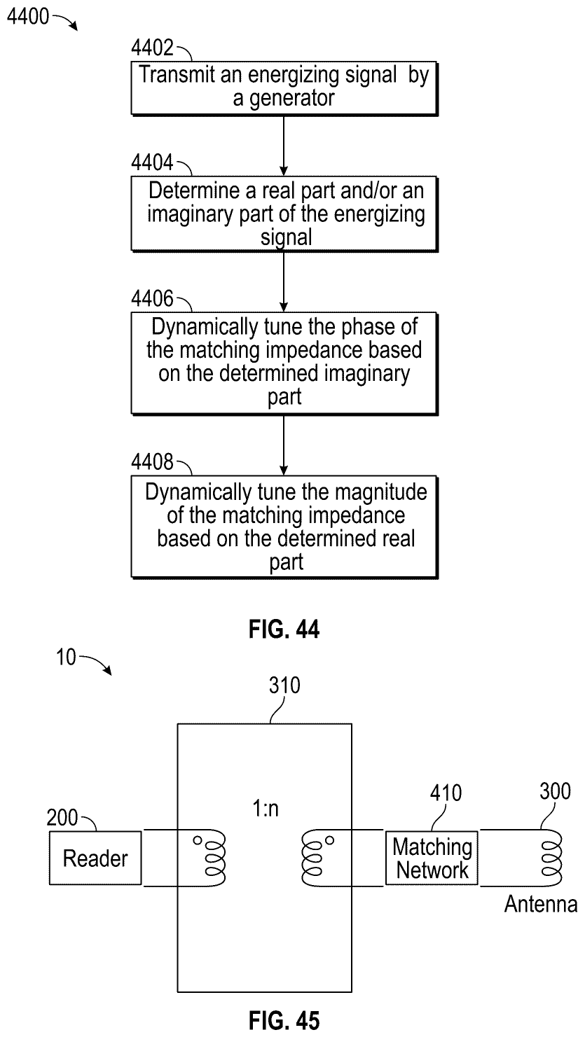

Referring to FIG. 44, a flow diagram for a method for real-time dynamically tuning an impedance match between an antenna 300 and a generator 200 is shown.

Referring now to FIG. 44, there is shown a flow diagram of a computer-implemented method 4400 for real-time dynamically tuning an impedance match between an antenna 300 and a generator 200. Persons skilled in the art will appreciate that one or more operations of the method 4400 may be performed in a different order, repeated, and/or omitted without departing from the scope of the disclosure. In various embodiments, the illustrated method 4400 can operate in the controller 2240 (FIG. 38), in a remote device, or in another server or system. In various embodiments, some or all of the operations in the illustrated method 4800 can operate using a surgical system such as the interrogation and detection system 10 of FIG. 1. Other variations are contemplated to be within the scope of the disclosure. The operations of FIG. 44 will be described with respect to a controller, e.g., controller 2240 (FIG. 38), but it will be understood that the illustrated operations are applicable to other systems and components thereof as well.

Initially, at step 4402, the signal generator 4402 transmits an energizing signal to an antenna 300, to which it is operably coupled. The energizing signal is configured to energize an RFID tag 100 (FIG. 1).

Next, at step 4404, the controller 2240 determines a real part and/or an imaginary part of the energizing signal by a tuning discriminator 3802 of the system 3800, which is configured to generate signals used for determining the real part (e.g., a current and/or a voltage) and/or imaginary part (e.g., a phase) of the energizing signal. For example, the real part of the energizing signal may be about 10.2 V.

Next, at step 4406, the controller 2240 dynamically tunes a phase of a matching impedance based on the determined imaginary part of the energizing signal. The controller 2240 may tune the phase of the matching impedance based on a phase compensation network 3840.

Next, at step 4408, the controller 2240 dynamically tunes a magnitude of a matching impedance based on the determined real part of the energizing signal. The controller 2240 may tune the magnitude of the matching impedance based on a magnitude compensation network 3840.

Referring to FIG. 45, a transformer 310 for matching an output impedance of a generator 200 to an impedance of an antenna 300 is shown. Generally, the output of the generator will be 50 ohms, and the impedance of the antenna 300 at or about the frequency of operation will be approximately 50 ohms. If both the generator 200 and the antenna 300 are set at a 50 ohms impedance, then the maximum amount of energy possible will be transferred. If the antenna 300 is not well matched to the impedance of the generator 200, a lower amount of power will be transferred. It may be desirable to use antennae where the impedance does not equal 50 ohms due to geometry, construction, or outside interference. To correct this, a matching network is typically placed between the antenna and the generator which transforms the antenna impedance to 50 ohms. The disclosed technology enables this matching and hence the construction and use of more effective antenna geometries.

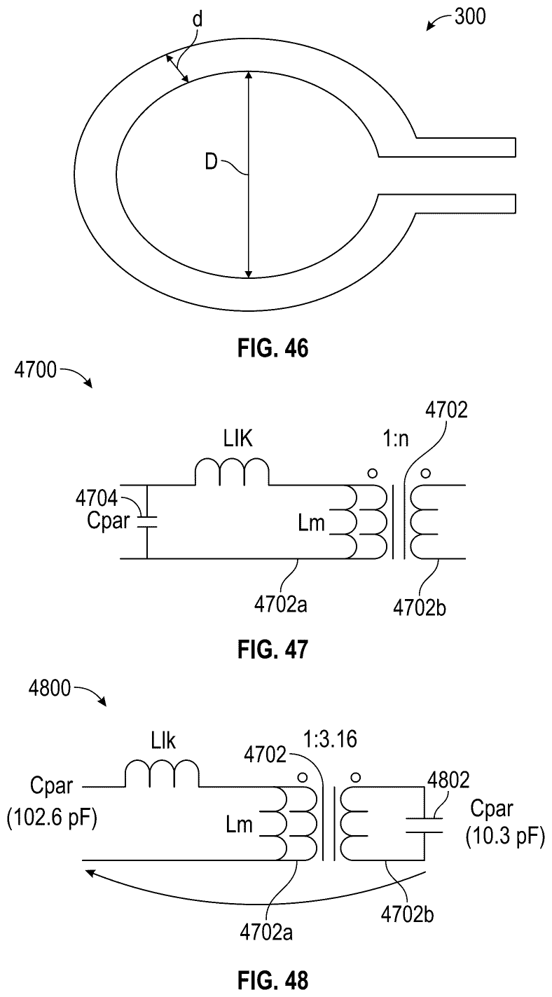

Referring to FIG. 46 a loop antenna 300, which may be used as the antenna for the detection system 10 (FIG. 45) is shown in accordance with the disclosure. The impedance for an antenna is based on the geometry of the antenna. The inductance for a loop antenna as shown in

FIG. 46 can be calculated using the following loop inductance equation:

$${L}_{loop} \approx {u}_{o} {u}_{r} \frac{D}{2} [ ln ( \frac{8 D}{d} ) - 2 ]$$

Where “Lₗₒₒₚ” is the loop inductance, “D” is the inner diameter of the loop, “d” is the width of the loop conductor, “uᵣ” is the relative permeability, and “uₒ” is the permeability of free space.

For example, in a case where an antenna inductance is approximately 1.174 uH, using the loop inductance equation, then for a 1 cm wide copper conductor, the diameter of the antenna would calculate out to be about 47.5 cm. This is a fairly large antenna. However, it may be desirable to have an even larger antenna or to have one with more turns. This could lead to antennas with inductances about ten times or twenty times the value that gives a 50 ohm matched load. Generally, the use of a matching network with this large an inductance may result in unusable component values or simply not be possible.

As the inductance of the antenna increases, the smaller the capacitance that is used in the matching network. Thus, making the loop antenna increasingly difficult to manufacture, and the effectiveness of the antenna may be reduced. For example, for a matching network for a 5.87 uH antenna, the matching network is approximately a 6.9 pF capacitor in parallel with the loop antenna. Thus, at this low a level of capacitance, it is entirely possible the manufacturing process may result in a parasitic capacitance that is greater than this value without even adding a discrete component (e.g., a discrete capacitor). This could be the result of cable parasitics or the parasitic capacitance present in the turns of a multi-turn antenna.

This disclosed technology addresses this issue through the use of a transformer. Adding a transformer element between the tuning network and the reader (e.g., the generator output) will allow the development of a higher impedance matching network that can then be transformed down to the 50 ohms that the reader is expecting. In aspects, the transformer will change the impedance of the matching network by the square root of the “turns ratio.”

ZₚZₛ/n²

For example, in the case of the matching network above, the impedance of the matching network transforms down to 50 ohms. In a case where the impedance on the primary of the transformer is 50 and on the secondary the impedance is 500 ohms, then a transformer with a “turns ratio” (N) of \sqrt{10} or 3.16 is required.

Although, typically represented as an ideal circuit that simply translates voltages, currents, and impedances to other levels, practical transformers have additional parasitic components that can play a real role in the way a circuit works. At a minimum, these parasitics must be accounted for, while in a well-designed circuit, the parasitic components can be used as part of the impedance matching network.

Impedance matching networks come in many different topologies. They can have series inductances, parallel inductances, or parallel capacitances or other configurations. Transformers can be used to replace the discrete impedance matching network components if they are carefully designed.

Referring to FIG. 47, a schematic of a transformer model 4700 including parasitics is shown. The transformer model 4700 generally includes the transformer 4702, parallel capacitance (Cpar) 4704 of the windings, leakage inductance (Llk), and mutual inductance of the windings (Lm). The transformer 4702 includes a primary winding 4702a and a secondary winding 4702b.

Referring to FIG. 48, a schematic of a transformer model 4800, including reflecting the parasitic capacitance, is shown. The transformer model 4800 generally includes the transformer 4702, parallel capacitance (Cpar) 4804 of the windings, leakage inductance (Llk), and mutual inductance of the windings (Lm). The transformer 4702 includes a primary winding 4702a and a secondary winding 4702b.

The equation for reflecting a capacitance through a transformer is: Cₚᵣᵢ=n²C_{sec}. This means that any capacitance placed on the secondary winding 4702b of the transformer 4702 will get multiplied by the “turns ratio” squared (N²) on the primary. In the case of the previous example, N²=10, where the “turns ratio” N is 3.16. Thus, a relatively small capacitance of 10.3 pF on the primary is needed to achieve our matching network component. For example, a capacitance of 10.3 pF could be realized by a discrete component and/or by embedded printed circuit board capacitance. A benefit of this approach is that leakage inductance and magnetizing inductance will be reduced as they are reflected through the transformer 4702.

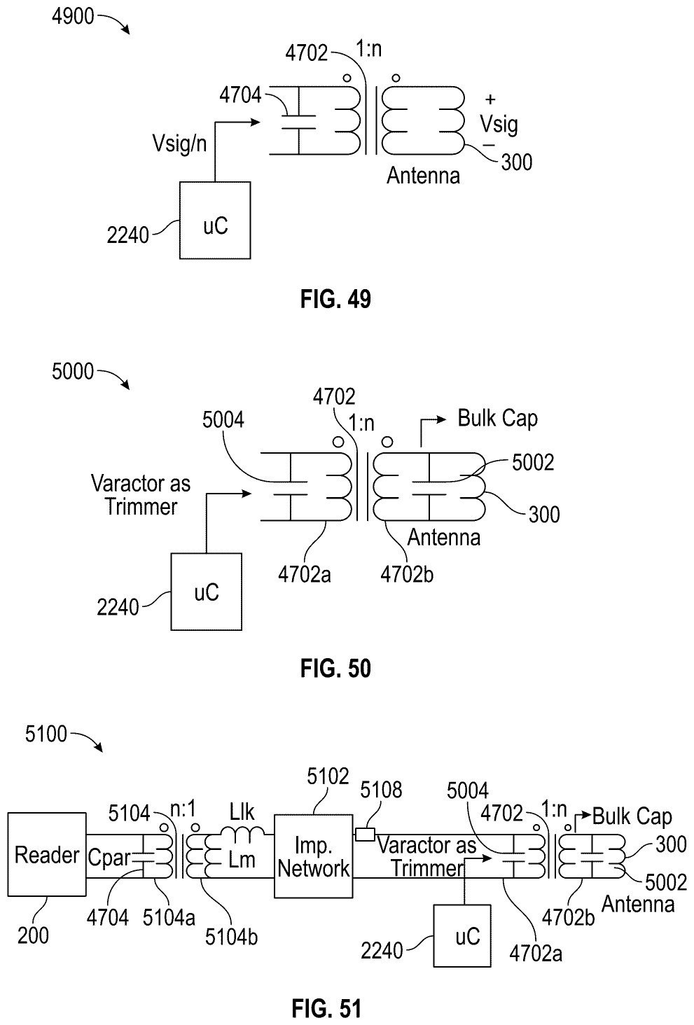

Referring to FIG. 49, a depiction of a transformer matching system 4900 for use with the interrogation and detection system of FIG. 1 is shown. The transformer matching system 4900 incorporates a dynamic capacitive element 4704 as a parallel capacitance to tune the resonance of the antenna 300. The system 4900 includes a transformer 4702, a dynamic capacitive element 4704, and a controller 2240. The transformer 4704 is configured to provide voltage protection for the tuning capacitor 4704

In aspects, the system 4900 enables dynamically tuning of an antenna resonance 410 (FIG. 45). The system 4900 reflects the capacitance of the dynamic capacitive element 4704 (e.g., a varactor) through a transformer 4702. This enables, for example, a low voltage varactor to be used in a high voltage system. For example, the signal received by the antenna 300 may reach as high as approximately 1 kV. If that is reflected down through a 1:10 transformer, the signal size would only be 100V. This way, the dynamic capacitive element 4704 could be used to dynamically tune the antenna 300 while staying within the voltage limitations of the dynamic capacitive element 4704 (e.g., 300V for a varactor).

Referring to FIG. 50, is a depiction of a transformer matching system 5000 that incorporates a dynamic capacitive element 4704 for a parallel capacitance, in accordance with the disclosure. The system 5000 generally includes a transformer 4702, a high voltage bulk capacitance 5002 on the secondary winding 4702b of the transformer 4702, and a dynamic capacitive element 5004 on the primary winding 4702a of the transformer 4702.

The dynamic capacitive element 5004 is configured to provide a “trimming” capacitance to help fine tune the antenna resonance 300. The dynamic capacitive element 5004 is an element that acts as a capacitive reactance and is tunable by an analog and/or digital signal. The dynamic capacitive element 5004 may include a varactor. For example, if the dynamic capacitive element 5004 is on the primary winding 4702a of the transformer 4702, then the value of the dynamic capacitive element 5004 will be reduced by “turns ratio” squared (N²) as it gets reflected through the transformer 4702. For example, if the dynamic capacitive element 5004 was to have a capacitance range of about 200 pF and “turns ratio” squared (N²)=10, then on the secondary winding, the range would only be 20 pF. If our turning requirements called for 100 pF, then this dynamic capacitive element 5004 would not be able to tune the circuit. If instead an approximately 90 pF high voltage bulk capacitance was used on the secondary winding 4702b, then the tunable range of the circuit would be about 90 pF to about 110 pF.

In aspects, the controller 2240 may be configured to control the value of the dynamic capacitive element 5004. For example, the controller 2240 may control a tuning voltage of the dynamic capacitive element 5004 to tune the resonance of the antenna 300.

Referring to FIG. 51, a system 5100 for matching an impedance of a generator 200 to an antenna 300 is shown. The system 5100 generally includes a first transformer 5104, an impedance matching network 5102, a dynamic capacitive element 5004, a second transformer 4702, an antenna 300, and a controller 2240.

The first transformer 5104 may be configured to transform the typical 50 ohm impedance of the generator 200 to an impedance other than 50 ohms. In aspects, the first transformer 5104 may include a step-up transformer or a step-down transformer. Careful design will allow the use of transformer parasitics to be part of the matching network. For example, if the parasitic capacitor 4704 was to have a capacitance of about 20 pF and the first transformer 5104 has a N²=10, then on the secondary winding 5104b the capacitance would be about 200 pF which may be an appropriate value for the matching network.

The matching network 5102, may include a fixed matching network (e.g., fixed capacitor and/or inductor values) and/or may include a dynamic matching network 2250 (FIG. 22). The matching network 5102 is disposed between the first transformer 5104 and the second transformer 4702. The matching network 510 is configured to match the impedance of the output of the generator 200 to the impedance of antenna 300. In aspects, the system 5100 may include a sensor 5108 configured to determine a parameter of the energizing signal. In aspects, the dynamic matching network 5102 may be configured to dynamically match the impedance between the signal generator and the antenna based on a parameter of the energizing signal (e.g., a power level, a frequency, a bandwidth, a voltage, and/or a current). The sensor 5105 may include, for example, in aspects, the sensor may be disposed in any suitable location in the circuit, for example, before and/or after the matching network 5102, at the output of the signal generator 200, and/or on a feed of the antenna 300.

The dynamic capacitive element 5004 is disposed on a primary winding 4702a of the second transformer 4702. The dynamic capacitive element 5004 is configured to provide a tunable capacitance for the antenna resonance whose voltage exposure is limited by the transformer. The controller 2240 may be configured to generate a signal for tuning the capacitance value of the dynamic capacitive element 5004.

The second transformer 4702 is configured to transform the impedance of the dynamic capacitive element 5102 to create the appropriate antenna resonance 300. In aspects, the system may further include a bulk capacitance 5002 configured to adjust the tuning range of the dynamic capacitive element 5004.

The antenna 300 may include a coil antenna, a loop antenna, an antenna array, and/or any other suitable antenna arrangement. For example, the system 5100, may be used to match a dipole antenna to a signal generator 200. In aspects, the second transformer may be a step-down transformer.

Further Aspects of the present disclosure include the following examples:

Example 1. An interrogation and detection system for detection of surgical implements within a body of a patient, the interrogation and detection system comprising:
an RFID tag configured to transmit a return signal when energized, the RFID tag affixed to a surgical implement;
a signal generator configured to generate an energizing signal for the RFID tag; and
a coil antenna operably coupled to the signal generator, the antenna configured to receive a return signal transmitted by the RFID tag, the coil antenna including:
   a primary coil;
   a secondary coil; and
   a core configured to couple electromagnetic energy from the secondary coil to the primary coil, wherein the core includes a non-magnetic insulating material,
   wherein the secondary coil is configured to receive the return signal, and
   wherein the primary coil is configured to couple electromagnetic energy to and from the secondary coil.

Example 2. The system of Example 1, wherein a “turns ratio” between the primary coil and the secondary coil is greater than or equal to 1:1, wherein the “turns ratio” is a ratio between a first quantity of turns of the primary coil and a second quantity of turns of the secondary coil.

Example 3. The system of Example 1, wherein the secondary coil includes a resonant frequency within 10% of an operating frequency of the return signal.

Example 4. The system of Example 1, wherein the secondary coil includes an inductance greater than or equal to 2.5 uH.

Example 5. The system of Example 1, wherein the coil antenna further includes a first matching network electrically connected to the primary coil, wherein the first matching network is configured to match an input impedance of the primary coil to an output impedance of the signal generator.

Example 6. The system of Example 1, wherein the coil antenna further includes a second matching network electrically connected to the secondary coil, wherein the second matching network is configured to match an input impedance of the secondary coil to an output impedance of the primary coil.

Example 7. The system of Example 1, wherein the primary coil is a first planar coil.

Example 8. The system of Example 7, wherein the secondary coil is a second planar coil.

Example 9. The system of Example 7, wherein the primary coil includes a first turn and a second turn.

Example 10. The system of Example 9, wherein the first turn of the primary coil and the second turn of the primary coil are arranged in an offset manner in a vertical and a horizontal orientation.

Example 11. The system of Example 8, wherein the secondary coil includes a first turn and a second turn.

Example 12. The system of Example 11, wherein the first turn of the primary coil and the second turn of the primary coil are arranged in an offset manner in a vertical and a horizontal orientation.

Example 13. A coil antenna configured to receive a return signal transmitted by an RFID tag, the coil antenna comprising:
a primary coil;
a secondary coil; and
a core configured to couple electromagnetic energy from the secondary coil to the primary coil, wherein the core includes a non-magnetic insulating material,
wherein the secondary coil is configured to receive the return signal, and
wherein the primary coil is configured to couple electromagnetic energy from the secondary coil.

Example 14. The coil antenna of Example 13, wherein the primary coil and the secondary coil are each planar coils.

Example 15. The coil antenna of Example 13, wherein the primary coil includes a conductor.

Example 16. The coil antenna of Example 15, wherein the conductor of the primary coil includes a conductor having a configuration including at least one of coaxial, planar, a “C” shaped transverse cross-sectional shape, or tube shaped transverse cross-sectional shape.

Example 17. The coil antenna of Example 13, wherein a “turns ratio” between the primary coil and the secondary coil is greater than or equal to 1:1, wherein the “turns ratio” is a ratio between a first quantity of turns of the primary coil and a second quantity of turns of the secondary coil.

Example 18. The coil antenna of Example 13, wherein the secondary coil includes a resonant frequency within 10% of an operating frequency of the return signal.

Example 19. A method for inventory control of tagged items, the method comprising:
transmitting an energizing signal by a coil antenna operably coupled to a signal generator, the coil antenna including a primary coil, a secondary coil, and a core configured to magnetically couple the secondary coil to the primary coil, the energizing signal configured to energize an RFID tag affixed to an item and configured to transmit a return signal when energized; and
receiving a return signal, by a primary coil of the coil antenna.

Example 20. The method of Example 19, further comprising:
detecting or identifying the item based on the return signal, wherein the item includes a surgical implement.

Example 21. A coil configured to receive a return signal transmitted by an RFID tag, the coil comprising:
a first turn conductor; and
a second turn conductor electrically connected to the first turn conductor.

Example 22. The coil of Example 21, wherein the first turn conductor is located in parallel relation and an offset manner to the second turn conductor,
wherein the first turn conductor includes a first inner edge and a first outer edge, and
wherein the second turn conductor includes a second inner edge and a second outer edge.

Example 23. The coil of Example 21, wherein the coil is arranged in at least one of a circular, square, rectangular, or oblong configuration.

Example 24. The coil of Example 21, wherein the first turn conductor overlaps the second turn conductor of the staggered coil, where a substantial portion of the second turn conductor is not overlapped by the first turn conductor.

Example 25. A coil antenna configured to receive a return signal transmitted by an RFID tag, the coil antenna comprising:
a primary coil; and
a secondary coil including:
   a first turn conductor; and
   a second turn conductor electrically connected to the first turn conductor,
wherein the secondary coil is configured to receive the return signal, and
wherein the primary coil is configured to couple electromagnetic energy from the secondary coil.

Example 26. The coil antenna of Example 25, further comprising a core configured to couple electromagnetic energy from the secondary coil to and from the primary coil, wherein the core includes a non-magnetic insulating material,
wherein the first turn conductor is located in parallel relation and an offset manner to the second turn conductor.

Example 27. The coil antenna of Example 25, wherein a “turns ratio” between the primary coil and the secondary coil is greater than or equal to 1:1, wherein the “turns ratio” is a ratio between a first quantity of turns of the primary coil and a second quantity of turns of the secondary coil.

Example 28. The coil antenna of Example 25, wherein the secondary coil includes a resonant frequency within 10% of an operating frequency of the return signal.

Example 29. The coil antenna of Example 25, wherein the secondary coil includes an inductance greater than or equal to 2.5 uH.

Example 30. The coil antenna of Example 25, wherein the coil antenna further includes a first matching network electrically connected to the primary coil, wherein the first matching network is configured to match an input impedance of the primary coil to an output impedance of the signal generator.

Example 31. An interrogation and detection system for detection of surgical implements within a patient's body, the interrogation and detection system comprising:
an RFID tag configured to transmit a return signal when energized, the RFID tag affixed to a surgical implement within the patient's body;
a signal generator configured to generate an energizing signal for the RFID tag; and
a coil antenna operably coupled to the signal generator, the antenna configured to receive a return signal transmitted by the RFID tag, the coil antenna including:
   a primary coil; and
   a secondary coil including:
      a first turn conductor; and
      a second turn conductor electrically connected to the first turn conductor,
      wherein the first turn conductor is located in parallel relation and an offset manner to the second turn conductor,
   wherein the secondary coil is configured to receive the return signal, and
   wherein the primary coil is configured to couple electromagnetic energy to and from the secondary coil.

Example 32. The system of Example 31, wherein the secondary coil is air-core coupled to the primary coil.

Example 33. The system of Example 31, wherein a “turns ratio” between the primary coil to the secondary coil is greater than or equal to 1:1, wherein the “turns ratio” is a ratio between a first quantity of turns of the primary coil and a second quantity of turns of the secondary coil.

Example 34. The system of Example 31, wherein the secondary coil includes an inductance greater than or equal to 2.5 uH.

Example 35. The system of Example 31, wherein the coil antenna further includes a first matching network electrically connected to the primary coil.

Example 36. The system of Example 31, wherein the coil antenna further includes a second matching network electrically connected to the secondary coil.

Example 37. The system of Example 31, wherein the primary coil is a first planar coil.

Example 38. The system of Example 31, wherein the secondary coil is a second planar coil.

Example 39. The system of Example 37, wherein the primary coil includes one or more turns.

Example 40. The system of Example 38, wherein the secondary coil includes one or more turns.

Example 41. An interrogation and detection system for detection of surgical implements within a patient's body, the interrogation and detection system comprising:
an RFID tag configured to transmit a return signal when energized, the RFID tag affixed to a surgical implement within the patient's body;
a signal generator configured to generate an energizing signal for the RFID tag; and
a coil antenna operably coupled to the signal generator, the antenna configured to receive a return signal transmitted by the RFID tag, the coil antenna including a coil array.

Example 42. The system of Example 41, wherein the coil array is configured to generate a magnetic flux and steer at least one of a direction of the magnetic flux or a magnitude of the magnetic flux based on the energizing signal.

Example 43. The system of Example 41, wherein the coil array includes a first coil and a second coil,
wherein the energizing signal includes a first current and a second current, and
wherein the first coil and the second coil are configured to independently be energized by the first current and the second current respectively.

Example 44. The system of Example 43, wherein the second coil is oriented at least one of 0, 90, 180, or 270 degrees relative to the first coil.

Example 45. The system of Example 44, wherein the first coil of the coil array is energized with the first current in at least one of a clockwise direction or a counter-clockwise direction, and
wherein the second coil of the coil array is energized with the second current in at least one of a clockwise direction or a counter-clockwise direction.

Example 46. The system of Example 43, wherein each coil of the coil array is arranged in at least one of a circular, square, rectangular, or oblong configuration.

Example 47. The system of Example 43, wherein each of the first coil and the second coil are planar coils.

Example 48. The system of Example 43, wherein each of the first coil and the second coil include one or more turns.

Example 49. The system of Example 41, wherein each of the coils of the coil array include a primary coil and a secondary coil,
and wherein each of the coils of the coil array further include a “turns ratio” between the primary coil to the secondary coil greater than or equal to 1:1, wherein the “turns ratio” is a ratio between a first quantity of turns of the primary coil and a second quantity of turns of the secondary coil.

Example 50. A coil array configured to receive a return signal transmitted by an RFID tag, the coil array comprising:
a first coil configured to generate a first magnetic field; and
a second coil configured to generate a second magnetic field,
wherein the coil array is configured to generate a magnetic flux and steer at least one of a direction of the magnetic flux or a magnitude of the magnetic flux based on an energizing signal from a signal generator.

Example 51. The coil array of Example 50, wherein the energizing signal includes a first current and a second current, and
wherein the first coil and the second coil are configured to independently be energized by the first current and the second current respectively.

Example 52. The coil array of Example 50, wherein the second coil is oriented at least one of 0, 90, 180, or 270 degrees relative to the first coil.

Example 53. The coil array of Example 52, wherein the first coil of the coil array is energized with the first current in at least one of a clockwise direction or a counter-clockwise direction, and
wherein the second coil of the coil array is energized with the second current in at least one of a clockwise direction or a counter-clockwise direction.

Example 54. The coil array of Example 51, wherein each coil of the coil array is arranged in at least one of a circular, square, rectangular, or oblong configuration.

Example 55. The coil array of Example 51, wherein each of the first coil and the second coil are planar coils.

Example 56. The coil array of Example 51, wherein each of the first coil and the second coil include one or more turns.

Example 57. The coil array of Example 50, wherein each of the coils of the coil array include a primary coil and a secondary coil,
and wherein each of the coils of the coil array further include a “turns ratio” between the primary coil to the secondary coil greater than or equal to 1:1, wherein the “turns ratio” is a ratio between a first quantity of turns of the primary coil and a second quantity of turns of the secondary coil.

Example 58. The coil array of Example 50, wherein each of the coils of the coil array includes an inductance greater than or equal to 2.5 uH.

Example 59. A method for interrogation and detection of surgical implements within a patient's body, the method comprising:
transmitting an energizing signal by a coil antenna operably coupled to a signal generator, the antenna configured to receive a return signal transmitted by an RFID tag, the coil antenna including a coil array; and
receiving a return signal, by the coil array, wherein the coil array is configured to generate a magnetic flux and steer at least one of a direction of the magnetic flux or a magnitude of the magnetic flux based on the energizing signal.

Example 60. The method of Example 59, wherein the energizing signal includes a first current and a second current, and
wherein the method further includes:
energizing, by the signal generator, a first coil of the coil array is energized by the first current in at least one of a clockwise direction or a counter-clockwise direction, and
energizing, by the signal generator, a second coil of the coil array by the second current in at least one of a clockwise direction or a counter-clockwise direction.

Example 61. A system for matching an impedance between an antenna and a signal generator, the system comprising:
an RFID tag configured to transmit a return signal when energized, the RFID tag affixed to a surgical implement within a patient's body;
the signal generator configured to generate an energizing signal for the RFID tag;
a coil antenna operably coupled to the signal generator, the antenna configured to receive a return signal transmitted by the RFID tag; and
a first transformer configured to match the impedance between the signal generator and the antenna.

Example 62. The system of Example 61, wherein the first transformer includes a primary winding and a secondary winding,
wherein the system further comprises a capacitor disposed in parallel to the primary winding of the first transformer, the capacitor is configured to match the impedance between the signal generator and the antenna.

Example 63. The system of Example 61, further comprising a matching network disposed between the first transformer and the antenna, and the matching network is configured to match the impedance between the signal generator and the antenna.

Example 64. The system of Example 63, wherein the matching network includes at least one of a fixed matching network or a dynamic matching network, wherein the dynamic matching network is configured to dynamically match the impedance between the signal generator and the antenna based on a parameter of the energizing signal.

Example 65. The system of Example 64, wherein the parameter of the energizing signal includes at least one of a power level, a frequency, a bandwidth, a voltage, or a current.

Example 66. The system of Example 63, further comprising a second transformer configured to transform an impedance match between the matching network and the antenna, the second transformer is disposed between the matching network and the antenna.

Example 67. The system of Example 66, wherein the second transformer is a step up transformer.

Example 68. The system of Example 67, further comprising a dynamic capacitive element configured to tune the impedance, the dynamic capacitive element is disposed across the primary winding of the second transformer.

Example 69. The system of Example 68, further comprising:
a sensor configured to sense a signal indicative of a parameter of the energizing signal;
a processor; and
a memory, including instructions stored thereon, which when executed cause the system to:
   sense the signal indicative of a parameter of the energizing signal;
   determine parameter of the energizing signal based on the sensed signal; and
   dynamically tune the dynamic capacitive element based on the determined parameter of the energizing signal.

Example 70. The system of Example 66, wherein the second transformer includes a primary winding and a secondary winding, and
wherein the system further comprises a bulk capacitance disposed across the secondary winding of the second transformer.

Example 71. The system of Example 66, wherein the second transformer is a step down transformer.

Example 72. A system for matching an impedance between an antenna and a signal generator, the system comprising:
an RFID tag configured to transmit a return signal when energized, the RFID tag affixed to a surgical implement within a patient's body;
the signal generator configured to generate an energizing signal for the RFID tag;
the antenna operably coupled to the signal generator, the antenna configured to receive a return signal transmitted by the RFID tag;
a transformer configured to match the impedance between the signal generator and the antenna; and
a dynamic capacitive element configured to tune the impedance, the dynamic capacitive element is disposed across the primary winding of the transformer.

Example 73. The system of Example 72, further comprising:
a sensor configured to sense a signal indicative of a parameter of the energizing signal;
a processor; and
a memory, including instructions stored thereon, which when executed cause the system to:
   sense the signal indicative of a parameter of the energizing signal;
   determine parameter of the energizing signal based on the sensed signal; and
   dynamically tune the dynamic capacitive element based on the determined parameter of the energizing signal.

Example 74. The system of Example 73, wherein the parameter of the energizing signal includes at least one of a power level, a frequency, a bandwidth, a voltage, or a current.

Example 75. The system of Example 73, wherein the transformer includes a primary winding and a secondary winding, and
wherein the system further comprises a bulk capacitance disposed across the secondary winding of the transformer.

Example 76. The system of Example 72, wherein the transformer is a step down transformer.

Example 77. The system of Example 72, further comprising a matching network disposed between the transformer and the antenna, and the matching network is configured to match the impedance between the signal generator and the antenna.

Example 78. The system of Example 77, wherein the matching network includes at least one of a fixed matching network or a dynamic matching network, wherein the dynamic matching network is configured to dynamically match the impedance between the signal generator and the antenna based on a parameter of the energizing signal.

Example 79. A method for tuning an impedance match between an antenna and a signal generator, the method comprising:
sensing, by a sensor, a signal indicative of a parameter of an energizing signal;
determining parameter of the energizing signal based on the sensed signal; and
dynamically tuning a dynamic capacitive element based on the determined parameter of the energizing signal, wherein the dynamic capacitive element is disposed across a primary of a transformer disposed between the signal generator and the antenna.

Example 80. The method of Example 79, wherein the parameter of the energizing signal includes at least one of a power level, a frequency, a bandwidth, a voltage, or a current.

Example 81. A system for real-time dynamically tuning an impedance match between an antenna and a signal generator, the system comprising:
an RFID tag configured to transmit a return signal when energized, the RFID tag affixed to a surgical implement within a patient's body;
a signal generator configured to generate an energizing signal for the RFID tag;
a coil antenna operably coupled to the signal generator, the antenna configured to receive a return signal transmitted by the RFID tag; and
a real-time tuning network configured to dynamically tune the impedance match between the signal generator and the antenna.

Example 82. The system of Example 81, wherein the real-time tuning network includes:
a tuning discriminator configured to determine at least one of a real part of the energizing signal or an imaginary part of the energizing signal;
a phase compensation network configured to dynamically tune a phase of the matching impedance based on the determined imaginary part of the energizing signal; and
a magnitude compensation network configured to dynamically tune a magnitude of the matching impedance based on the determined real part of the energizing signal,
wherein the imaginary part includes at least one of a capacitive signal, an inductive signal, or a composite signal, and
wherein the real part includes at least one of an energizing signal current or an energizing signal voltage.

Example 83. The system of Example 82, wherein the real-time tuning discriminator includes:
an impedance transform network configured to transform the energizing signal from the signal generator for identification of the real part and the imaginary part of the energizing signal;
a rectifier configured to rectify the impedance transformed signal; and
a low pass filter configured to filter the rectified signal.

Example 84. The system of Example 82, wherein the real-time tuning network further includes a power detector configured to detect at least one of the energizing signal current or the energizing signal voltage.

Example 85. The system of Example 82, wherein the phase compensation network includes:
a dynamic capacitive element configured to select a frequency range for the phase of the matching impedance; and
a transformer configured to reduce a voltage across the dynamic capacitance element.

Example 86. The system of Example 82, wherein the magnitude compensation network includes:
a dynamic capacitive element configured to select a frequency range of the magnitude of matching impedance; and
a transformer configured to reduce a voltage across the dynamic capacitance element.

Example 87. The system of Example 83, further comprising:
a processor; and
a memory, including instructions stored thereon, which, when executed, cause the system to:
   determine an imaginary part of the energizing signal; and
   dynamically tune the phase of the matching impedance based on the determined imaginary part of the energizing signal by the phase compensation network.

Example 88. The system of Example 87, wherein the instructions when executed further cause the system to:
determine a real part of the energizing signal; and
dynamically tune, by the magnitude compensation network, the magnitude of the matching impedance based on the determined real part of the energizing signal of the energizing signal.

Example 89. The system of Example 82, wherein the coil antenna includes:
a primary coil; and
a secondary coil.

Example 90. The system of Example 88, wherein the tuning network is disposed between the primary coil and the signal generator, and
wherein the tuning network is configured to tune at least one of a quality factor “Q” or an operating frequency of the primary coil.

Example 91. The system of Example 89, further comprising a second tuning network electrically coupled to the secondary coil and configured to tune at least one of a quality factor “Q” or an operating frequency of the secondary coil.

Example 92. A method for real-time dynamically tuning an impedance match between an antenna coil and a signal generator, the method comprising:
determining at least one of a real part of an energizing signal of the signal generator or an imaginary part of an energizing signal of the signal generator; and
dynamically tuning, by a phase compensation network, a phase of the matching impedance based on the determined imaginary part of the energizing signal.

Example 93. The method of Example 92, further comprising:
determining a real part of the energizing signal; and
dynamically tuning, by a magnitude compensation network, a magnitude of the matching impedance based on the determined real part of the energizing signal of the energizing signal.

Example 94. A real-time tuning network configured to dynamically tune an impedance match between a signal generator and an antenna, the real-time tuning network comprising:
a real-time tuning discriminator configured to determine at least one of a real part of an energizing signal from the signal generator or an imaginary part of the energizing signal;
a phase compensation network configured to dynamically tune a phase of the matching impedance based on the determined imaginary part of the energizing signal; and
a magnitude compensation network configured to dynamically tune a magnitude of the matching impedance based on the determined real part of the energizing signal,
wherein the imaginary part includes at least one of a capacitive signal, an inductive signal, or a composite signal, and
wherein the real part includes at least one of an energizing signal current or an energizing signal voltage.

Example 95. The real-time tuning network of Example 94, wherein the real-time tuning discriminator includes:
an impedance transform network configured to transform the energizing signal from the signal generator for identification of the real part and the imaginary part of the energizing signal;
a rectifier configured to rectify the impedance transformed signal; and
a low pass filter configured to filter the rectified signal.

Example 96. The real-time tuning network of Example 95, wherein the real-time tuning network further includes a power detector configured to detect at least one of the energizing signal current or the energizing signal voltage.

Example 97. The real-time tuning network of Example 95, wherein the phase compensation network includes:
a dynamic capacitive element configured to select a frequency range for the phase of the matching impedance; and
transformer configured to reduce a voltage across the dynamic capacitance element.

Example 98. The real-time tuning network of Example 94, wherein the magnitude compensation network includes:
a dynamic capacitive element configured to select a frequency range of the magnitude of matching impedance; and
a transformer configured to reduce a voltage across the dynamic capacitance element.

Example 99. The real-time tuning network of Example 95, wherein the coil antenna includes:
a primary coil; and
a secondary coil.

Example 100. The real-time tuning network of Example 98, wherein the tuning network is disposed between the primary coil and the signal generator,
wherein the tuning network is configured to tune at least one of a quality factor “Q” or an operating frequency of the primary coil.

Example 101. A system for dynamically configuring a secondary air-core coupled coil and exciting magnetic fields, the system comprising:
an RFID tag configured to transmit a return signal when energized, the RFID tag affixed to a surgical implement within a patient's body;
a signal generator configured to generate an energizing signal for the RFID tag; and
a coil antenna operably coupled to the signal generator, the antenna configured to receive a return signal transmitted by the RFID tag, the coil antenna configured to excite a magnetic field in multiple directions based on the energizing signal.

Example 102. The system of Example 101, wherein the coil antenna includes:
a coil array including a plurality of coils, wherein each coil of the coil array includes:
   a primary coil; and
   a secondary coil.

Example 103. The system of Example 102, wherein the coil antenna further includes a coil tuning network configured to tune at least one of a quality factor “Q” or an operating frequency of the primary coil.

Example 104. The system of Example 103, wherein tuning network includes:
a real part match detection network configured to detect the real part of the energizing signal;
an imaginary part match detection network configured to detect the imaginary part of the energizing signal;
a dynamic matching network configured to tune at least one of a quality factor “Q” or a first resonant frequency of the primary coil;
a processor; and
a memory with instructions stored thereon, which when executed by the processor cause the system to:
   detect a real part of the energizing signal, by the real part match detection network;
   detect a real part of the energizing signal, by the imaginary part match detection network;
   determine a second resonant frequency for the primary coil, based on the detected real part and detected imaginary part of the energizing signal; and
   tune, by the dynamic matching network, the primary coil to the second resonant frequency, based on the determination.

Example 105. The system of Example 104, wherein tuning network further includes a power detection network configured to detect a power level from the energizing signal.

Example 106. The system of Example 105, wherein the instructions when executed further cause the system to:
detect, power detection network, the power level of the energizing signal;
determine a third resonant frequency for the primary coil; and
tune, by the dynamic matching network, the primary coil to the third resonant frequency, based on the determination.

Example 107. The system of Example 102, wherein the coil antenna further includes a termination network configured to enable or disable discrete secondary coils of the coil array.

Example 108. The system of Example 107, wherein the termination network includes:
an impedance sensor configured to sense the impedance of each of the secondary coils of the coil array;
a step down transformer configured to step down the impedance of each of the secondary coils of the coil array;
a dynamic capacitive bank configured to provide a plurality of loads to each of the secondary coils of the coil array via the step down transformer;
a processor; and
a memory with instructions stored thereon, which when executed by the processor cause the system to:
determine, by the impedance sensor, the impedance of the return signal; and
set the dynamic capacitive bank to one of the plurality of loads based on the determination.

Example 109. The system of Example 102, wherein the secondary coil is a configurable air-core coupled secondary coil, including a plurality of configurable secondary coil sections, the configurable air-core coupled secondary coil having a plurality of secondary coil configurations.

Example 110. The system of Example 109, wherein the coil antenna further includes a steering network configured to enable at least one of the plurality of secondary coil configurations.

Example 111. The system of Example 101, further including a surgical table, wherein the coil antenna is embedded into the surgical table.

Example 112. The system of Example 102, wherein the coil array includes a first coil and a second coil,
wherein the energizing signal includes a first current and a second current, and
wherein the first coil and the second coil are configured to independently be energized by the first current and the second current respectively.

Example 113. The system of Example 112, wherein the first coil of the coil array is energized with the first current in at least one of a clockwise direction or a counter-clockwise direction, and
wherein the second coil of the coil array is energized with the second current in at least one of a clockwise direction or a counter-clockwise direction.

Example 114. A coil antenna, comprising:
a coil array configured to receive a return signal transmitted by an RFID tag, comprising:
   a first coil configured to generate a first magnetic field; and
   a second coil configured to generate a second magnetic field,
   wherein the coil array including a plurality of coils configured to generate a magnetic flux and steer at least one of a direction of the magnetic flux or a magnitude of the magnetic flux based on an energizing signal from a signal generator, wherein each coil of the coil array includes:
      a primary coil; and
      a secondary coil.

Example 115. The coil antenna of Example 114, wherein the coil antenna further includes a coil tuning network configured to tune at least one of a quality factor “Q” or an operating frequency of each primary coil.

Example 116. The coil antenna of Example 114, wherein tuning network includes:
a real part match detection network configured to detect the real part of an energizing signal;
an imaginary part match detection network configured to detect the imaginary part of the energizing signal;
a dynamic matching network configured to tune at least one of a quality factor “Q” or a first operating frequency of the primary coil;
a processor; and
a memory with instructions stored thereon, which when executed by the processor cause the coil antenna to:
   detect a real part of the energizing signal, by the real part match detection network;
   detect a real part of the energizing signal, by the imaginary part match detection network;
   determine a second operating frequency for the primary coil, based on the detected real part and detected imaginary part of the energizing signal; and
   tune, by the dynamic matching network, the primary coil to the second operating frequency, based on the determination.

Example 117. The coil antenna of Example 114, wherein the coil antenna further includes a termination network configured to enable or disable discrete secondary coils of the coil array.

Example 118. The coil antenna of Example 117, wherein the termination network includes:
a sensor configured to sense at least one of an impedance, a voltage, or a current of each of the secondary coils of the coil array.

Example 119. The coil antenna of Example 114, wherein the secondary coil is a configurable air-core coupled secondary coil, including a plurality of configurable secondary coil sections, the configurable air-core coupled secondary coil having a plurality of secondary coil configurations.

Example 120. A method for interrogation and detection of surgical implements within a patient's body, the method comprising:
transmitting an energizing signal by a coil antenna operably coupled to a signal generator, the antenna configured to receive a return signal transmitted by an RFID tag, the coil antenna including a coil array;
receiving a return signal, by the coil array, wherein the coil array is configured to generate a magnetic flux and steer at least one of a direction of the magnetic flux or a magnitude of the magnetic flux based on the energizing signal;
detecting a real part of the energizing signal, by a real part match detection network;
detecting a real part of the energizing signal, by an imaginary part match detection network;
determining a second operating frequency for the primary coil, based on the detected real part and detected imaginary part of the energizing signal; and
tuning, by a dynamic matching network, the primary coil to the second operating frequency, based on the determination.

While aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

1-100. (canceled)

101. A system for dynamically configuring a secondary air-core coupled coil and exciting magnetic fields, the system comprising:
an RFID tag configured to transmit a return signal when energized, the RFID tag affixed to a surgical implement within a patient's body;
a signal generator configured to generate an energizing signal for the RFID tag; and
a coil antenna operably coupled to the signal generator, the antenna configured to receive a return signal transmitted by the RFID tag, the coil antenna configured to excite a magnetic field in multiple directions based on the energizing signal.

102. The system of claim 101, wherein the coil antenna includes:
a coil array including a plurality of coils, wherein each coil of the coil array includes:
a primary coil; and
a secondary coil.

103. The system of claim 102, wherein the coil antenna further includes a coil tuning network configured to tune at least one of a quality factor “Q” or an operating frequency of the primary coil.

104. The system of claim 103, wherein tuning network includes:
a real part match detection network configured to detect the real part of the energizing signal;
an imaginary part match detection network configured to detect the imaginary part of the energizing signal;
a dynamic matching network configured to tune at least one of a quality factor “Q” or a first resonant frequency of the primary coil;
a processor; and
a memory with instructions stored thereon, which when executed by the processor cause the system to:
detect a real part of the energizing signal, by the real part match detection network;
detect a real part of the energizing signal, by the imaginary part match detection network;
determine a second resonant frequency for the primary coil, based on the detected real part and detected imaginary part of the energizing signal; and
tune, by the dynamic matching network, the primary coil to the second resonant frequency, based on the determination.

105. The system of claim 104, wherein tuning network further includes a power detection network configured to detect a power level from the energizing signal.

106. The system of claim 105, wherein the instructions when executed further cause the system to:
detect, power detection network, the power level of the energizing signal;
determine a third resonant frequency for the primary coil; and
tune, by the dynamic matching network, the primary coil to the third resonant frequency, based on the determination.

107. The system of claim 102, wherein the coil antenna further includes a termination network configured to enable or disable discrete secondary coils of the coil array.

108. The system of claim 107, wherein the termination network includes:
an impedance sensor configured to sense the impedance of each of the secondary coils of the coil array;
a step down transformer configured to step down the impedance of each of the secondary coils of the coil array;
a dynamic capacitive bank configured to provide a plurality of loads to each of the secondary coils of the coil array via the step down transformer;
a processor; and
a memory with instructions stored thereon, which when executed by the processor cause the system to:
determine, by the impedance sensor, the impedance of the return signal; and
set the dynamic capacitive bank to one of the plurality of loads based on the determination.

109. The system of claim 102, wherein the secondary coil is a configurable air-core coupled secondary coil, including a plurality of configurable secondary coil sections, the configurable air-core coupled secondary coil having a plurality of secondary coil configurations.

110. The system of claim 109, wherein the coil antenna further includes a steering network configured to enable at least one of the plurality of secondary coil configurations.

111. The system of claim 101, further including a surgical table, wherein the coil antenna is embedded into the surgical table.

112. The system of claim 102, wherein the coil array includes a first coil and a second coil,
wherein the energizing signal includes a first current and a second current, and
wherein the first coil and the second coil are configured to independently be energized by the first current and the second current respectively.

113. The system of claim 112, wherein the first coil of the coil array is energized with the first current in at least one of a clockwise direction or a counter-clockwise direction, and
wherein the second coil of the coil array is energized with the second current in at least one of a clockwise direction or a counter-clockwise direction.

114. A coil antenna, comprising:
a coil array configured to receive a return signal transmitted by an RFID tag, comprising:
a first coil configured to generate a first magnetic field; and
a second coil configured to generate a second magnetic field,
wherein the coil array including a plurality of coils configured to generate a magnetic flux and steer at least one of a direction of the magnetic flux or a magnitude of the magnetic flux based on an energizing signal from a signal generator, wherein each coil of the coil array includes:
a primary coil; and
a secondary coil.

115. The coil antenna of claim 114, wherein the coil antenna further includes a coil tuning network configured to tune at least one of a quality factor “Q” or an operating frequency of each primary coil.

116. The coil antenna of claim 114, wherein tuning network includes:
a real part match detection network configured to detect the real part of an energizing signal;
an imaginary part match detection network configured to detect the imaginary part of the energizing signal;
a dynamic matching network configured to tune at least one of a quality factor “Q” or a first operating frequency of the primary coil;
a processor; and
a memory with instructions stored thereon, which when executed by the processor cause the coil antenna to:
detect a real part of the energizing signal, by the real part match detection network;
detect a real part of the energizing signal, by the imaginary part match detection network;
determine a second operating frequency for the primary coil, based on the detected real part and detected imaginary part of the energizing signal; and
tune, by the dynamic matching network, the primary coil to the second operating frequency, based on the determination.

117. The coil antenna of claim 114, wherein the coil antenna further includes a termination network configured to enable or disable discrete secondary coils of the coil array.

118. The coil antenna of claim 117, wherein the termination network includes:
a sensor configured to sense at least one of an impedance, a voltage, or a current of each of the secondary coils of the coil array.

119. The coil antenna of claim 114, wherein the secondary coil is a configurable air-core coupled secondary coil, including a plurality of configurable secondary coil sections, the configurable air-core coupled secondary coil having a plurality of secondary coil configurations.

120. A method for interrogation and detection of surgical implements within a patient's body, the method comprising:
transmitting an energizing signal by a coil antenna operably coupled to a signal generator, the antenna configured to receive a return signal transmitted by an RFID tag, the coil antenna including a coil array;
receiving a return signal, by the coil array, wherein the coil array is configured to generate a magnetic flux and steer at least one of a direction of the magnetic flux or a magnitude of the magnetic flux based on the energizing signal;
detecting a real part of the energizing signal, by a real part match detection network;
detecting a real part of the energizing signal, by an imaginary part match detection network;
determining a second operating frequency for the primary coil, based on the detected real part and detected imaginary part of the energizing signal; and
tuning, by a dynamic matching network, the primary coil to the second operating frequency, based on the determination.
